(19) United States
(12) Patent Application Publication
(10) Pub. No.: US 2025/0345796 A1
(43) Pub. Date: Nov. 13, 2025

(54) DIGITAL CHROMATOGRAPHY MICROFLUIDIC BIOCHIP AND METHODS OF USE THEREOF

(71) Applicant: The University of Hong Kong, Hong Kong (CN)

(72) Inventors: Zhiting Peng, Hong Kong (CN); Yau Kei Chan, Hong Kong (CN); Ho Cheung Shum, Hong Kong (CN); Yi Hao Wu, Hong Kong (CN); Wei Guo, Hong Kong (CN)

(21) Appl. No.: 19/193,621

(22) Filed: Apr. 29, 2025

Related U.S. Application Data
(60) Provisional application No. 63/643,773, filed on May 7, 2024.

(51) Int. Cl. B01L 3/00; G01N 33/543

(52) U.S. Cl. CPC B01L 3/502761; B01L 3/502707; B01L 3/502715; G01N 33/54326; B01L 2200/026; B01L 2300/0663; B01L 2300/0819; B01L 2300/0883; B01L 2300/161

(57) ABSTRACT

Compositions and methods for quantitating target molecules from samples using digital chromatography implemented on microfluidic biochips are described. A microfluidic device including two symmetric microfluidic channels, each incorporating hydrophobic filter structures and high-density hydrophilic flow-trap junction arrays (FT-JA) is provided. Centrally positioned turbine valves increase the resistance in the flow channel directing the fluid laterally through the trap channel. The microfluidic device, e.g., a chip, is configured to facilitate the simultaneous, parallel capture of control and test samples including a target molecule, e.g., a biomarker, immobilized on microscale particles, e.g., microbeads, by capturing the beads in the FT-JA. In some forms, a microfluidic chip quantifies biomarkers within a biological sample with 90% efficiency for imaging within a compact area, e.g., 30 mm², in a low time frame, e.g., 80 seconds. Exemplary biomarkers that can be quantified according to the described methods include tumor antigens and biomarkers derived from pathogens. Exemplary samples include tears, plasma and blood.

### CROSS-REFERENCE TO RELATED APPLICATIONS

This application claims the benefit of and priority to U.S. Provisional Application No. 63/643,773 filed May 7, 2024, which is hereby incorporated by reference in its entirety.

### FIELD OF THE INVENTION

The present invention relates to the automated detection of biomarkers in micro- and nano-scale samples, and in particular to the use of digital chromatography for high-sensitivity immunoassays.

### BACKGROUND OF THE INVENTION

The precise quantification of protein biomarkers at ultralow concentrations has emerged as a cornerstone for early disease diagnosis and personalized therapeutic strategies. While conventional enzyme-linked immunosorbent assays (ELISAs) remain ubiquitous in clinical practice, their continuous analog signal detection imposes fundamental sensitivity limits, particularly for detecting scarce biomarkers in minute biological samples (e.g., tear fluid, cerebrospinal fluid). The advent of digital ELISA revolutionized molecular diagnostics by enabling single-molecule counting through micro compartmentalization, achieving attomolar sensitivity by converting continuous signals into discrete digital events. This paradigm relies on antibody-coated magnetic beads to capture target molecules, which are subsequently isolated in microwells or droplets containing chemiluminescent substrates for individual signal quantification. Despite achieving remarkable sensitivity, current digital ELISA platforms employing magnetic beads encounter several persistent obstacles.

Firstly, the opacity and refractive index of magnetic bead cores (Fe₃O₄, n=2.42) substantially attenuate fluorescence intensity, reducing detectable signal intensities by approximately three-fold compared with silica beads (SiO₂, n=1.45). This optical interference fundamentally limits signal-to-noise ratios and spatial resolution in full-field imaging. Secondly, magnetic bead-based methods exhibit limited capture efficiencies ranging from 40% to 50% within complex biological samples, as shown in prior studies Simoa® HD-1 Analyzer. Low microbead detection rates (10-30%) further diminish assay effectiveness due to limited imaging throughput and restricted bead array densities. Thirdly, magnetic field-induced bead aggregation frequently leads to nonspecific interactions and incomplete purification, which collectively reduce assay specificity and precision. Environmental variables such as pH fluctuations, thermal variations, and nonspecific molecular adsorption further complicate accurate biomarker quantification, demanding meticulous calibration procedures and standardized assay conditions. These limitations are compounded by statistical constraints inherent to Poisson distribution assumptions, in which scientists typically estimate the overall concentration of molecules by analyzing the frequency of positive signals among microbeads (AMB, average molecule per bead). Although increasing the fraction of analyzed beads (analytical fraction >80%) theoretically improves measurement precision by reducing counting errors and enhancing statistical reliability (% CV=√n/n), practical implementation remains hindered by limitations in imaging throughput. Conversely, reducing bead usage adversely impacts the kinetics of molecular interactions (kₒₙ∝[bead]²), thus compromising both dynamic range and practical feasibility. Consequently, detection windows become narrow due to signal saturation at elevated concentrations (typically >100 molecules/bead) and heightened stochastic noise at lower extremes, limiting overall measurement precision. For specimens with bimodal concentration distributions (e.g., 0.1 fM & 0.1 nM co-existing), sparse bead arrays fail simultaneous detection (sensitivity ratio >1000×).

Recent strategies have sought to address these challenges by adjusting capture efficiencies, and assay methodologies. Microdroplet-based approaches, for example, demonstrate superior capture efficiencies (up to 95%), although this advantage often comes at the expense of complexity in handling their imaging due to reduced array density. Conversely, assays employing fewer beads achieve greater enzyme occupancy per bead, intensifying signals but at the cost of prolonged incubation periods (up to three hours), highlighting the intrinsic trade-offs between dynamic range, and throughput. While the drop-cast assay improves the handling efficiency, their reliance on evaporation-mediated assembly introduces environmental instability that limits clinical utility.

In parallel, conventional lateral flow immunoassays (LFIA) offer rapid, magnetic-free operation through straightforward immunochromatographic principles. Biological samples migrate along a nitrocellulose membrane via capillary action, interacting with antibody-conjugated nanoparticles to produce visually discernible colored bands at test and control regions. However, LFIA reliance on subjective colorimetric interpretations significantly constrains their quantitative precision and sensitivity. Even with reader devices or enhanced labels, conventional LFIAs typically achieve limits of detection in the high picomolar to nanomolar range (around 0.1-10 ng/mL) and rarely below this range. Immunochromatography (ICA), known for its expeditious, straightforward, and dependable nature, is widely utilized in point-of-care testing, negating the need for sophisticated instrumentation or specialized laboratory settings. In the immunochromatographic process, the introduction of the fluid sample onto the membrane strip allows for the binding of the target molecule (antigen) to labeled antibodies present on the strip, resulting in the appearance of visible colored lines in both test and control regions.

While the visual detection of colored lines on the test strip enables qualitative analysis, the method is limited by the subjective interpretation of color intensity, which is especially problematic in the quantitative determination of biomarkers present in trace amounts.

Thus, there exists a need for compositions and methods for biomarker detection and analysis that are more efficient, more automatable, have enhanced sensitivity, have enhanced reproducibility, require less sample, and require lower concentrations of biomarkers.

Therefore, it is an object of the invention to provide systems and methods for automated detection of biomarkers with high-sensitivity.

It is also an object of the invention to provide systems and methods for the simultaneous detection of multiple biomarkers from a single sample.

It is also an object of the invention to provide methods to simplify assay procedures and increase throughput and scalability for diagnostic systems.

It is also an object of the invention to provide methods for reliable analysis of biological samples that are not influenced by variations in environmental factors such as changes in pH, temperature, ionic concentration and interfacial effects.

It is a further object of the invention to provide methods reducing background noise and enhance detection using low sample volumes and/or low concentration so target biomarkers to enhance diagnoses of disease and disorders.

### SUMMARY OF THE INVENTION

Compositions and methods for the scalable, automated, detection of biomarkers using microfluidics systems have been developed. The compositions and methods employ Digital Immunochromatography (ICA) to implement conventional ICA with the enhanced sensitivity found in digital ELISA. Methods for clinical diagnostics by detecting protein biomarkers in small-volume samples are also provided.

Disclosed are microfluidic chips and methods of making and using the disclosed chips. The microfluidic chips generally include a microfluidic platform including one or more microfluidic flow paths. In some forms, the microfluidic flow paths include two inlet conduits, a flow-trap junction (FTJ) array structure, and two outlet conduits. In some forms, the microfluidic flow path is configured for movement of fluid from the inlet conduit into the FTJ structure, and from the FTJ structure into the outlet conduit.

In some forms, the inlet conduit is wider where the fluid moves from the inlet conduit into the FTJ structure than where the fluid is introduced into the inlet conduit. In some forms, the inlet conduit contains a multiplicity of hydrophobic micropillar structures.

In some forms, the FTJ structure includes a flow layer and a trap layer. In some forms, the flow layer is in contact with, on top of, and overlapping with the trap layer.

In some forms, the flow layer includes a plurality of flow microfluidic channels each including a top, side walls, and an opening on the bottom. In some forms, the surfaces of the flow microfluidic channels are hydrophobic. In some forms, the flow microfluidic channels allow free passage of micro-scale particles and nano-scale objects. In some forms, the fluid flows in the same direction in all of the flow microfluidic channels.

In some forms, the flow microfluidic channels are parallel to each other. In some forms, the trap layer includes a plurality of trap microfluidic channels each including a bottom, side walls, and an opening on the top. In some forms, the trap microfluidic channels are parallel to each other. In some forms, the surfaces of the trap microfluidic channels are hydrophilic. In some forms, the fluid flows in the same direction in all of the trap microfluidic channels.

In some forms, the flow microfluidic channels are not parallel to the trap microfluidic channels. In some forms, the flow microfluidic channels and the trap microfluidic channels allow fluid movement from the flow microfluidic channels into the trap microfluidic channels via the openings on the bottom and openings on the top, respectively.

In some forms, reverse-oriented channels positioned at the terminus of each flow channel create turbine valve structures, with centrally located valves in the flow layer inducing controlled hydrodynamic resistance to direct fluid laterally into the trap network. The system facilitates selective bead capture through an adaptive zigzag pathway dynamically governed by trap occupancy states: unoccupied traps exhibit minimal resistance, enabling bead ingress and settling, whereas occupied traps generate elevated resistance profiles, redirecting fluid recirculation to primary channels and steering subsequent beads toward adjacent available sites. This self-regulating process ensures spatially ordered bead deposition, as fluid dynamics autonomously adjust to real-time trap availability. Upon saturation of all trapping sites, surplus beads undergo controlled evacuation via the terminal flow conduit, preventing overcrowding while maintaining optimal particle density. The architecture operates through autonomous fluidic regulation, leveraging occupancy-dependent resistance modulation, self-directed particle routing, and systemic overfill prevention to achieve non-mechanical flow control, self-limiting deposition, and scalable array formation without reliance on external control systems or active monitoring components.

In some forms, the trap microfluidic channels, the transition from the flow microfluidic channels to the trap microfluidic channels, or a combination of both the trap microfluidic channels and the transition from the flow microfluidic channels to the trap microfluidic channels are configured to allow passage of the nano-scale objects through the trap microfluidic channels, whereby the passaged nano-scale objects flow into the outlet conduit.

In some forms, the trap microfluidic channels, the transition from the flow microfluidic channels to the trap microfluidic channels, or a combination of both the trap microfluidic channels and the transition from the flow microfluidic channels to the trap microfluidic channels are configured to trap the micro-scale particles in the trap microfluidic channels, whereby the trapped micro-scale particles, in combination, form an array within the FTJ structure.

In some forms, the side walls of the trap microfluidic channels are straight and parallel to each other, where the height of the trap microfluidic channels is less than the diameter of the micro-scale particles. In some forms, the height of the trap microfluidic channels is more than the diameter or long dimension of the nano-scale objects.

In some forms, the side walls of the trap microfluidic channels are not straight such that the trap microfluidic channels vary in width in a regular pattern along their lengths. In some forms, the pattern of width variation forms narrowings in the width of the trap microfluidic channels, where the width of the narrowings are less than the diameter of the micro-scale particles. In some forms, the width of the narrowings are more than the diameter or long dimension of the nano-scale objects.

In some forms, all or a subset of the narrowings overlap the flow layer between some or each of the openings on the bottoms of adjacent flow microfluidic channels. In some forms, all or a subset of the narrowings overlap the opening on the bottom of some or each of the flow microfluidic channels. In some forms, a subset of the narrowings overlap the opening on the bottom of some or each of the flow microfluidic channels and a subset of the narrowings overlap the flow layer between some or each of the openings on the bottoms of adjacent flow microfluidic channels. In some forms, a subset of the narrowings overlap the opening on the bottom of each of the flow microfluidic channels and a subset of the narrowings overlap the flow layer between each of the openings on the bottoms of adjacent flow microfluidic channels.

In some forms, the narrowings overlapping the openings on the bottoms of the flow microfluidic channels form a small trap entrance on the down-flow side of the opening and a large trap entrance on the down-flow side of the opening for alternating trap microfluidic channels. In some forms, the size of the small trap entrance is less than the diameter of the micro-scale particles. In some forms, the size of the small trap entrance is more than the diameter or long dimension of the nano-scale objects. In some forms, the size of the large trap entrance is more than the diameter of the micro-scale particles.

In some forms, the flow microfluidic channels and the trap microfluidic channels are at a right angle to each other. In some forms, the flow microfluidic channels and the trap microfluidic channels are at an oblique angle to each other. In some forms, the flow microfluidic channels and the trap microfluidic channels are at an angle of between 60° to 90°, between 60° to 90°, between 60° to 90°, between 60° to 90°, between 60° to 90°, between 70° to 90°, between 80° to 90°, between 85° to 90°, between 87° to 90°, between 88° to 90°, or between 89° to 90°, to each other.

In some forms, the side walls of the trap microfluidic channels are angled toward the up-flow ends of the flow microfluidic channels.

In some forms, the microfluidic flow path also includes a sample inlet. In some forms, the microfluidic flow path is configured for movement of fluid from the sample inlet into the inlet conduit.

In some forms, the microfluidic flow path also includes a plurality of outlet channels, wherein the microfluidic flow path is configured for movement of fluid from the trap microfluidic channels into the outlet channels and from the outlet channels into the outlet conduit. In some forms, each trap microfluidic channel is flowably connected to a different one of the outlet channels.

In some forms, the flow layer of the FTJ structure also includes a plurality of outlet channels. In some forms, the outlet channels are interspersed between and parallel to the flow microfluidic channels. In some forms, the outlet channels each include a top, side walls, and an opening on the bottom. In some forms, the outlet channels and the trap microfluidic channels allow fluid movement from the trap microfluidic channels into the outlet channels via the opening on the bottom and openings on the top, respectively. In some forms, the microfluidic flow path is configured for movement of fluid from the trap microfluidic channels into the outlet channels and from the outlet channels into the outlet conduit.

In some forms, the outlet channels alternate with the flow microfluidic channels in the flow layer of the FTJ structure.

In some forms, the flow layer of the FTJ structure also includes an outlet channel. In some forms, the outlet channel includes a top, side walls, and an opening on the bottom. In some forms, the outlet channel overlaps the down-flow ends of the trap microfluidic channels. In some forms, the outlet channel and the trap microfluidic channels allow fluid movement from the trap microfluidic channels into the outlet channel via the opening on the bottom and openings on the top, respectively. In some forms, the microfluidic flow path is configured for movement of fluid from the trap microfluidic channels into the outlet channel and from the outlet channel into the outlet conduit.

In some forms, the chip is transparent in one or more regions. In some forms, the chip is transparent in a region corresponding to the array. In some forms, the array includes a surface area of 25 mm² or less.

In some forms, the microfluidic platform includes two microfluidic flow paths. In some forms, the two microfluidic flow paths are flowably connected to a single fluid reservoir. In some forms, the fluid reservoir is flowably connected to the respective inlet conduits of the two microfluidic flow paths.

In some forms, the two microfluidic flow paths are symmetrically disposed on the microfluidic platform. In some forms, the two microfluidic flow paths are symmetrically disposed on the chip.

In some forms, the microfluidic chip also includes a plurality of micro-scale particles. In some forms, the micro-scale particle includes a microbead. In some forms, the microbead includes a magnetic microbead.

In some forms, the micro-scale particles also include a first capture agent specific for a target biomarker. In some forms, the first capture agent is conjugated to the micro-scale particle via streptavidin. In some forms, one or more of the first capture agents are bound to the target biomarker.

In some forms, the micro-scale particles have a diameter of between about 1 μm and about 5 μm, inclusive. In some forms, each of the micro-scale particles have between about 200,000 and about 400,000 first capture agents, inclusive.

In some forms, the first capture agent is selected from the group consisting of an antibody, a nucleic acid, a protein, a lipid, a carbohydrate, and a small molecule. In some forms, the first capture agent includes DNA or RNA, or both.

In some forms, the micro-scale particles are located within the array. In some forms, the micro-scale particles are located within the array at a density of about 100 micro-scale particles/μm². In some forms, the array includes from about 1×10⁴ micro-scale particles to about 1×10⁶ micro-scale particles, inclusive. In some forms, the array includes about 4×10⁵ micro-scale particles. In some forms, the array has an area of from about 10 mm² to about 50 mm², inclusive, optionally about 25 mm².

In some forms, the microfluidic chip includes a plurality of nano-scale objects. In some forms, the nano-scale objects include a second capture agent specific for the target biomarker. In some forms, one of the second capture agents are bound to one or more of the target biomarkers bound to the first capture agents.

In some forms, the nano-scale objects also include a reporter molecule. In some forms, the reporter molecule includes a highly bright quantum dot nanoparticle.

In some forms, the micropillars span the height of the inlet conduit. In some forms, the trap microfluidic channels include a hydrophilic polymer. In some forms, the hydrophilic polymer includes polyethylene glycol (PEG).

Disclosed are methods for detecting a target biomarker in a fluid sample. The method generally includes (a) introducing the fluid sample to one or more of the microfluidic flow paths of a microfluidic chip as disclosed herein and (b) performing digital chromatography on the chip. In some forms, the fluid sample includes, or is bought into contact with after its introduction, a plurality of micro-scale particles and a plurality of nano-scale objects. In some forms, digital chromatography identifies the presence and/or quantity of the target biomarker in the fluid sample.

In some forms, step (a) also includes introducing to a different microfluidic flow path of the same chip a control sample including a known amount of the target biomarker.

In some forms, the performance of digital chromatography of step (b) includes actuating movement of fluid through the microfluidic flow paths in the microfluidic chip. In some forms, the movement filters and washes the micro-scale particles within the FTJ structure. In some forms, the filtering of the micro-scale particles in the FTJ structure traps, and forms an array of, the micro-scale particles within the FTJ structure.

In some forms, step (b) also includes imaging the array of micro-scale particles within the microfluidic chip.

In some forms, the micro-scale particles includes a microbead. In some forms, the microbead includes a magnetic microbead. In some forms, the micro-scale particles also include a first capture agent specific for a target biomarker. In some forms, the first capture agent is conjugated to the micro-scale particle via streptavidin. In some forms, one or more of the first capture agents are bound to the target biomarker.

In some forms, the nano-scale objects include a second capture agent specific for the target biomarker. In some forms, one of the second capture agents is bound to one or more of the target biomarkers bound to the first capture agents.

In some forms, the nano-scale objects also include a reporter molecule. In some forms, the reporter molecule includes a highly bright quantum dot nanoparticle.

In some forms, step (b) also includes detecting and measuring the target biomarkers bound to the first capture agents on the micro-scale particles within the array.

In some forms, the method also includes, prior to step (a), (i) incubating the fluid sample with the micro-scale particles for a time and in an amount effective for binding of the target biomarkers to the first capture agent. In some forms, following step (i), washing the micro-scale particles.

In some forms, the method also includes, prior to step (a), contacting the micro-scale particles with the nano-scale objects for a time and in an amount effective for binding of the target biomarkers to the second capture agent.

In some forms, steps (a) and/or (b) include a total time of between about 10 and 1000 about seconds, inclusive, optionally about 180 seconds.

In some forms, the method detects at least 90%, such as 91%, 92%, 93%, 94%, 95%, 96%, 97%, 98%, 99%, or 100% of the biomarkers within the fluid sample. In some forms, the fluid sample includes a biomarker at a concentration of about 1 IU/mL. In some forms, the fluid sample includes a biomarker at a concentration of about 10⁻¹⁸ to 10⁻⁵ M. In some forms, the fluid sample includes a volume of between about 1.0 μL to about 100 μL, inclusive, optionally about 50 μL.

In some forms, the fluid sample includes between one and 100 different biomarkers, optionally between one and 10 different species of biomarkers. In some forms, one biomarker is an immunoglobulin. In some forms, one biomarker is an IgE. In some forms, one biomarker is an interferon. In some forms, one biomarker is TNF-alpha.

In some forms, the target biomarker within the fluid sample is derived from a bodily fluid from a subject. In some forms, the bodily fluid is selected from the group including blood, sweat, semen, serum, bile, saliva, tear fluid, pus, mucus, pleural fluid, vitreous fluid, spinal fluid, synovial fluid, amniotic fluid and urine. In some forms, the bodily fluid is tear fluid.

### BRIEF DESCRIPTION OF THE DRAWINGS

FIGS. 1A-1E are images that provide an overview of single-molecule protein detection with BiZi-FIA. FIG. 1A is a schematic of a BiZi-FIA chip, showing a detection area (FT-JA) compliant with maximum objective field of view limitation of 6×6 mm². FIG. 1B depicts Bright field microscopy image of microbeads array, imaging area is 30 mm². Images are obtained in two conduits to identify the “on” beads, and the “off” beads. FIG. 1C is a diagram of a BiZi-FIA chip structure and functionality, showing the two-layer composition of the BiZi-FIA chip, including the hydrophobic Flow layer (top), including both a test (T) and control (C) areas both connected to the same flow input via a double-lumen catheter; and the hydrophilic trap layer (bottom). FIG. 1D is an expanded view of the hydrophobic Flow layer depicted in FIG. 1C, showing the direction of fluid flowing within and throughout the test samples and the control area. FIG. 1E is a flowchart depicting the testing area with test samples and the control area, respectively, for an exemplary assay that includes (i) Filtering, whereby particles navigate through a filtering fractal structure including micropillars—this structure facilitates the constant collision of microbeads with the micropillars, leading to the dissociation of non-specifically adsorbed nanoparticles—the micro-pillars, featuring nonspecific adsorption properties with hydrophobic surface, capture and filter out the nanoparticles; (ii) washing and arraying occurs as particles pass into the traps; Modified with PEG reagent, the trap layer exhibits hydrophilic properties in the FT-JA area—this modification is instrumental in the elution of nonspecific nanoparticles, ensuring the selective retention of target microbeads-single protein molecules are captured on microbeads and labelled with ultrabright fluorescent nanoparticles (Qt dots labels). The control area features a structure that is a mirror reflection of the test area. By manipulating a single variable, the control area conducts tests in control samples; and (iii) determination of test sample concentration occurs as fluorescence signals detected in the FT-JA are considered background noise, guiding an adaptive algorithm to minimize system errors; a control concentration is input and a test sample concentration is output. A key for the flowchart is at bottom.

FIGS. 2A-2I depict a schematic and mechanism of FT-JA for microbead capture. Schematic representation of the FT-JA setup showing the reservoir and vent ports is illustrated in FIG. 2A. Detailed structure of the FT-JA highlighting the flow (Rf) and trap (Rt) resistances is illustrated in FIG. 2B. Microscopic image of the sequential array showing microbeads that are captured by FT-JA is illustrated in FIG. 2C. The 3D illustration of the FT-JA demonstrates the capturing process of microbeads in the sequential array is illustrated in FIG. 2D; a schematic depicting the flow and movement of particles within each of the sequential and supplemental arrays is depicted in FIG. 2E, including a simulation of the turbine valves centrally positioned to induce controlled hydrodynamic resistance and direct fluid laterally into the network of vertical access conduits and horizontal drainage conduits within the trap. FIG. 2F depicts the height-limited trap mechanism effectively directs and captures microbeads within the flow conduit. As microbeads enter the flow conduit, Microbead-1 bypasses an occupied trap and continues along the path. Microbead-2 supplementary sinks into an empty trap conduit, followed by Microbead-3. Microbead-4, which exceeds the capture capacity, is expelled through the terminal flow conduit. Once all eligible traps are occupied, the main flow is redirected to the straight flow channels. Subsequent microbeads, unable to enter occupied traps, follow the main flow out of the device. Microscopic images of the sequential supplementary array illustrating the microbead capture process is illustrated in FIG. 2G. Diagrams of the flow of fluid and beads within the sequential (test) array is depicted in FIG. 2H, and within the supplemental array are depicted in FIG. 2I, respectively.

FIGS. 3A-3K show performance evaluation of the BiZi-FIA system. Depicted is a simulated pressure gradient driving bilateral zigzag flow across FT-JA (FIG. 3A); Cross-sectional view of flow channels with differing widths (FIG. 3B); Simulated 2D multi-zigzag flow paths (FIG. 3C); and 3D single-zigzag and a schematic of microbead trapping in wide and narrow flow channels (FIG. 3D). Microscopic images of bead arrays in each of wide (FIG. 3E), medium (FIG. 3F), and narrow (FIG. 3G) channels, respectively, demonstrating increasing array density with narrower channels. FIGS. 3H-3J are graphs showing capture efficiency (FIG. 3H), and array density (FIG. 3I) trends across flow channel widths, and variance analysis of microbead capture in bilateral arrays, indicating high stability across batches (FIG. 3J), respectively. Simulated 3D multi-zigzag flow paths are depicted in FIG. 3K.

FIGS. 4A-4C are schematic diagrams, showing the process of the BiZi-FIA platform for single-molecule detection with automatic filtering and washing mechanism. FIG. 4A is a low-chart depicting reagent preparation and incubation in the BiZi-FIA workflow. FIGS. 4B-C are BiZi-FIA schematics showing filtering and washing mechanisms, using hydrophobic and hydrophilic surfaces (FIG. 4B) for efficient filtering (FIG. 4C).

FIGS. 5A-5B are graphs of Silica beads-enhanced fluorescence for extensive field of view detection, showing optical intensity comparison at a wavelength of 611 nm between magnetic and silica beads, showing a marked increase in signal for silica beads (FIG. 5A); and fluorescence intensity comparison across various magnifications (4×, 10×, 20×, 40×) showing the performance of magnetic and silica beads in enhancing signal detection (FIG. 5B).

FIGS. 6A-6D show Analysis of BiZi-FIA platform sensitivity and array density under varying conditions. FIG. 6A is a schematic of the capture antibody labelling method with SA-biotin systems. FIG. 6B is a schematic of reverse setup for detection antibodies labelling method with SA-biotin system. FIGS. 6C-6D are graphs of 400,000 beads used, the variation of LOD with different fractions of beads analyzed, showing optimal performance with higher fractions and lower objective lens magnification (FIG. 6C); and the relationship between limit of detection (LOD) in attomoles (aM) and the number of beads used to demonstrate increased sensitivity with higher bead density (FIG. 6D). With the number of beads used increased above the capturing ability, a certain number of beads would be accelerated lost, which leads to a decrease in the fraction of microbeads analyzed.

FIGS. 7A-7I are graphs showing comparisons of sensitivities of silica beads based on BiZi-FIA chip washing and magnetic beads based on conventional magnetic washing. FIGS. 7A-7C show a Digital immunoassay calibration curves for IgE (FIG. 7A), TNF-α (FIG. 7B), NFL (FIG. 7C) BiZi-FIA; and FIGS. 7D-7F show a conventional Digital immunoassay calibration curves for each of IgE (FIG. 7D), TNF-α (FIG. 7E), and NFL (FIG. 7F), respectively. Dashed lines indicate the calculated limits of detection (LODs). FIGS. 7G-7I show comparison of signal to background ratios between silica beads and conventional magnetic beads for both the BiZi-FIA and digital Immunoassays for each of IgE (FIG. 7G), TNF-α (FIG. 7H), and NFL (FIG. 7I), respectively.

FIGS. 8A-8C are schematics depicting the Adaptive Differential Algorithm, showing the differential mean value theorem for signal interpretation in adaptive corrections. The fluorescence intensity versus concentration curve illustrates relationships between S₀ (background noise), S_{Q} (quantitation sample signal), S_{C} (control sample signal), and Sₜ (test sample signal). Gradient differentials (f′(CQ), f′(C0), f′(Q0)) derived from concentration changes are critical for adaptive corrections. The shaded region highlights the difference between fluorescence units for accurate gradient optimization (FIG. 8A); Examples of fluorescent signal distributions across different sample types for training the ADNC. The red fluorescence dots represent the raw emission signal captured from the samples, while the green circles indicate signals that surpass the certain threshold and are subsequently identified as valid events by software. Blank sample (S_{O}), quantitation sample (S_{Q}), control sample (S_{C}), and test samples (Sₜ₁, Sₜ₂, Sₜ₃) show varying signal distributions (FIG. 8B); and a flowchart that outlines the key steps of the ADNC algorithm, including input setup, signal switching, and gradient threshold validation, including a 3D loss function surface plot (L(x)) demonstrates the iterative gradient descent process for minimizing discrepancies in unknown test sample concentrations (x₁, x₂, x₃). The algorithm ensures optimal calibration for robust signal corrections and accurate concentration predictions (FIG. 8C).

FIGS. 9A-9J are graphs of TNF-α (FIG. 9A, FIG. 9B) and NFL (FIG. 9C, FIG. 9D) generated using ADNC software with known concentrations. Linearity and accuracy analysis of TNF-α (FIG. 9E, FIG. 9F) and NFL (FIG. 9G, FIG. 9H) measurements, showing strong correlations (R²>0.99) between spiked and measured concentrations. FIG. 9I shows Quantitative comparison between the ADNC algorithm and the Poisson-based algorithm shows the broader dynamic range achieved with the ADNC algorithm. FIG. 9J shows Combined biomarker analysis showing the concentration profiles of IgE, TNF-α and NFL across cases, highlighting their wide dynamic range and potential for clinical diagnosis of allergic conjunctivitis and related inflammatory conditions.

FIG. 10 is a flow chart for a fabricated microfluidic device for BiZi-FIA using conventional photolithography and soft lithography techniques. After the bonding of the trap layer and the flow layer, a hydrophilic and hydrophobic surface treatment is conducted. To differentiate between the hydrophobic filtration area at the inlet and the hydrophilic array area near the outlet, the hydrophilic reagent, will be injected from one of the outlets under sealed inlet conditions, reaching the symmetrical end of the outlet.

### DETAILED DESCRIPTION OF THE INVENTION

The disclosed chip's design incorporates overlapping hydrophobic and hydrophilic interfaces to enhance filtration and washing within the chip, thereby improving the signal-to-noise ratio and eliminating background noise. The superhydrophobic interfaces are strategically placed at the entrance of the array and the connected filtration micropillars, while the hydrophilic interfaces are situated at the center of the array and the exits connected to the array center.

The chip features a symmetric design of the testing and control areas, sharing the same washing buffer inlet on either side. This symmetry in structure facilitates simultaneous sample introduction and processing in both areas, allowing for integrated control and comparative analysis, which is a distinct approach in assay technology.

The biochip includes a two-layer design, where the upper layer has symmetrically positioned inlets leading to a particle bifurcation structure and superhydrophobic filtration micropillars. This design ensures uniform distribution and flow of particles in the array area. The lower layer includes a staggered honeycomb mesh structure, facilitating high-density particle array formation and alignment of upper and lower chip layers without high-precision alignment systems. This two-layer design with specific flow and distribution control is innovative and not straightforward for those skilled in the art.

The design of the chip allows both microbead-based antibody capture and direct modification on the chip surface, demonstrating versatility in application. It offers superior dispersion capabilities for the analytes and the ability to control single variables during testing. This versatility and control are unique in the context of immunoassays.

The chip's design allows for a stringent control of a single variable during the assay. It differentiates effective signals from background noise based on control area readings. If the background noise exceeds the error range of the standard curve, the test is considered invalid; otherwise, the effective signal is calculated by subtracting the background noise from the test signal. This method of signal differentiation and control is both innovative and non-obvious.

In summary, the aspects of this chip include integration of surface chemistries, structural design for filtration and washing, dual-layer architecture for particle control, selective surface modification, and versatile application in immunoassay technology with enhanced signal control and differentiation. These features collectively contribute to the described systems and methods' distinction from existing technologies.

### I. Definitions

The term “nucleotide” refers to a molecule that contains a base moiety, a sugar moiety and a phosphate moiety. Nucleotides are typically linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

The term “residue” of a chemical species refers to the moiety that is the resulting product of the chemical species in a particular reaction scheme or subsequent formulation or chemical product, regardless of whether the moiety is actually obtained from the chemical species. Thus, an ethylene glycol residue in a polymer refers to one or more —OCH₂CH₂O— units in the polymer, regardless of whether ethylene glycol was used to prepare the polyester. As another example, in a polymer of monomer subunits, the incorporated monomer subunits can be referred to as residues of the un-polymerized monomer.

The term “nucleotide analog” refers to a nucleotide which contains some type of modification to the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

The term “nucleotide substitute” refers to a nucleotide molecule having similar functional properties to nucleotides, but which does not contain a phosphate moiety. An exemplary nucleotide substitute is peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. It is also possible to link other types of molecules (conjugates) to nucleotides or nucleotide analogs to enhance for example, interaction with DNA. Conjugates can be chemically linked to the nucleotide or nucleotide analogs. Exemplary conjugates include but are not limited to lipid moieties such as a cholesterol moiety.

The terms “nucleic acid,” “polynucleotide,” and “oligonucleotide” are interchangeable and refer to a deoxyribonucleotide or ribonucleotide biopolymer, in linear or circular conformation, and in either single- or double-stranded form. For the purposes of the present disclosure, these terms are not to be construed as limiting with respect to the length of a biopolymer. The terms can encompass known analogues of natural nucleotides, as well as nucleotides that are modified in the base, sugar and/or phosphate moieties (e.g., phosphorothioate backbones, locked nucleic acid). In general and unless otherwise specified, an analogue of a particular nucleotide has the same base-pairing specificity; i.e., an analogue of A will base-pair with T. When double-stranded DNA is described, the DNA can be described according to the conformation adopted by the helical DNA, as either A-DNA, B-DNA, or Z-DNA. The B-DNA described by James Watson and Francis Crick is believed to predominate in cells, and extends about 34 Å per 10 bp of sequence; A-DNA extends about 23 Å per 10 bp of sequence, and Z-DNA extends about 38 Å per 10 bp of sequence.

In some cases, nucleotide sequences are provided using character representations recommended by the International Union of Pure and Applied Chemistry (IUPAC) or a subset thereof. IUPAC nucleotide codes include, A=Adenine; C=Cytosine; G=Guanine; T=Thymin; U=Uracil; R=A or G; Y=C or T; S=G or C; W=A or T; K=Gor T; M=A or C; B=C or G or T; D=A or G or T; H=A or C or T; V=A or C or G; N=any base; “.” or “-”=gap. In some forms the set of characters is (A, C, G, T, U) for adenosine, cytidine, guanosine, thymidine, and uridine respectively. In some forms the set of characters is (A, C, G, T, U, I, X, Ψ) for adenosine, cytidine, guanosine, thymidine, uridine, inosine, uridine, xanthosine, pseudouridine, respectively. In some forms the set of characters is (A, C, G, T, U, I, X, Ψ, R, Y, N) for adenosine, cytidine, guanosine, thymidine, uridine, inosine, uridine, xanthosine, pseudouridine, unspecified purine, unspecified pyrimidine, and unspecified nucleotide, respectively.

The terms “polypeptide,” “peptide,” and “protein” are used interchangeably to refer to a polymer of amino acid residues. The term also applies to amino acid polymers in which one or more amino acids are chemical analogues or modified derivatives of corresponding naturally-occurring amino acids.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or MEGALIGN (DNASTAR) software is used to align sequences. Those skilled in the art can determine appropriate parameters for measuring alignment, including any formulas needed to achieve maximal alignment over the full-length of the sequences being compared. When sequences are aligned, the percent sequence identity of a given sequence A to, with, or against a given sequence B (which can alternatively be phrased as a given sequence A that has or includes a certain percent sequence identity to, with, or against a given sequence B) can be calculated as: percent sequence identity=X/Y 100, where X is the number of residues scored as identical matches by the sequence alignment program's or formula's alignment of A and B and Y is the total number of residues in B. If the length of sequence A is not equal to the length of sequence B, the percent sequence identity of A to B will not equal the percent sequence identity of B to A. Mismatches can be similarly defined as differences between the natural binding partners of nucleotides. The number, position and type of mismatches can be calculated and used for identification or ranking purposes.

The phrase that a molecule “specifically binds” to a target refers to a binding reaction which is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. The term “specific binding”, for example, between two entities, means an affinity of at least 10⁶, 10⁷, 10⁸, 10⁹, or 10¹⁰ M-1. Affinities greater than 10⁸ M-1 are preferred.

The term “target molecule” refers to a substance which is desired, for example, to be detected and/or quantified from a mixture of molecules, including non-target molecules.

The terms “antibody” and “immunoglobulin” include intact antibodies, and binding fragments thereof. Typically, fragments compete with the intact antibody from which they were derived for specific binding to an antigen fragment, including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes a bispecific antibody. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin. Exp. Immunol., 79:315-321 (1990); Kostelny, et al., J. Immunol., 148, 1547-1553 (1992).

The terms “epitope” and “antigenic determinant” refer to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10, amino acids, in a unique spatial conformation. Methods of determining spatial conformation of epitopes include, for example, x-ray crystallography and 2-dimensional nuclear magnetic resonance.

The term “small molecule,” as used herein, generally refers to an organic molecule that is less than about 2,000 g/mol in molecular weight, less than about 1,500 g/mol, less than about 1,000 g/mol, less than about 800 g/mol, or less than about 500 g/mol. Small molecules are non-polymeric and/or non-oligomeric.

The term “bead” or “magnetic bead” refers to a solid structure that is used as a support matrix for one or more reagents when used in methods, for example, such as digital immunochromatography. Beads can be any suitable bead.

The terms “wash reagent,” “wash buffer,” “wash,” and “rinse solution” refer to a solution that is used to purify remove one or more reagents from a sample. Typically, the wash buffer is a solvent that is effective to solvate and remove reagents from a molecule that is immobilized, for example, an immobilized biomarker.

The term “wash conditions” refers to the environmental/external conditions under which combination with a wash reagent (i.e., a distinct “wash step”) is carried out. For example, a wash can be carried out by combining one or more wash reagents with a solution or immobilized support containing the biomarker.

The terms “microfluidic device”, “microfluidics”, “microfluidic chip”, and “microfluidic platform” refer to any device, or system that supports and/or enables or actuates the movement of sub-microliter volumes of fluids. Typically, microfluidic devices implement components and means for controlling the user-defined movement of fluid in a controlled manner, as well as modifying or altering one or more physicochemical properties, such as temperature, electric charge, light, magnetic force, etc. In some forms, microfluidic devices control the movement, behavior and manipulation of fluids through one or more means for actuating fluid movement. Exemplary microfluidic devices actuate fluid movements through mechanisms including continuous flow, fluid dispensing, EWOD, pressure, optical or combinations thereof. Microfluidic devices can be “open” (i.e., fluid is contained, moved and manipulated on a single surface), or “closed” (i.e., fluid is contained, moved and manipulated between two surfaces). In some forms, the term “microfluidic device” is used interchangeably with “microfluidic system”, and includes the means for inputting user-defined control of fluid manipulation (e.g., through a general-user interface that employs computer software to control the movement of fluids within the device). The term “microfluidic system” also refers to additional equipment, such as equipment that is external to apparatus for controlling fluid movement, for example, devices for controlling parameters such as temperature, light, pressure, humidity, etc. In some form, “microfluidic devices” include devices and systems to input data for control of the movement or manipulation of the droplets on a microfluidic platform located close to, or at a distance from the site of data input. In some forms, the data input device is or incorporates a computer. In some forms, the system or device includes one or more systems for providing information to the control system, e.g., a device for proving feedback. In some forms, data input is autonomous (e.g., computational tasks can be performed, autonomously, like programs that run on conventional silicon computers, but here in the liquid state).

Disclosed are microfluidic chips 100 and methods of making and using the disclosed chips. The microfluidic chips 100 generally include a microfluidic platform 110 including one or more microfluidic flow paths 120. In some forms, the microfluidic flow paths 120 include an inlet conduit 130, a flow-trap junction (FTJ) structure 140, and an outlet conduit 300. In some forms, the microfluidic flow path 120 is configured for movement of fluid 400 from the inlet conduit 130 into the FTJ structure 140, and from the FTJ structure 140 into the outlet conduit 300.

In some forms, the inlet conduit 130 is wider where the fluid 400 moves from the inlet conduit 130 into the FTJ structure 140 than where the fluid 400 is introduced into the inlet conduit 130. In some forms, the inlet conduit 130 contains a multiplicity of hydrophobic micropillar structures 135.

In some forms, the FTJ structure 140 includes a flow layer 150 and a trap layer 190. In some forms, the flow layer 150 is in contact with, on top of, and overlapping with the trap layer 190.

In some forms, the flow layer 150 includes a plurality of flow microfluidic channels 160 each including a top 162, side walls 164, and an opening 166 on the bottom. In some forms, the surfaces of the flow microfluidic channels 160 are hydrophobic. In some forms, the flow microfluidic channels 160 allow free passage of micro-scale particles 500 and nano-scale objects 600. In some forms, the fluid 400 flows in the same direction in all of the flow microfluidic channels 160.

In some forms, the flow microfluidic channels 160 are parallel to each other. In some forms, the trap layer 190 includes a plurality of trap microfluidic channels 200 each including a bottom 202, side walls 204, and an opening 206 on the top. In some forms, the trap microfluidic channels 200 are parallel to each other. In some forms, the surfaces of the trap microfluidic channels 200 are hydrophilic. In some forms, the fluid 400 flows in the same direction in all of the trap microfluidic channels 200.

In some forms, the flow microfluidic channels 160 are not parallel to the trap microfluidic channels 200. In some forms, the flow microfluidic channels 160 and the trap microfluidic channels 200 allow fluid 400 movement from the flow microfluidic channels 160 into the trap microfluidic channels 200 via the openings 166 on the bottom and openings 206 on the top, respectively.

In some forms, the trap microfluidic channels 200, the transition from the flow microfluidic channels 160 to the trap microfluidic channels 200, or a combination of both the trap microfluidic channels 200 and the transition from the flow microfluidic channels 160 to the trap microfluidic channels 200 are configured to allow passage of the nano-scale objects 600 through the trap microfluidic channels 200, whereby the passaged nano-scale objects 600 flow into the outlet conduit 300.

In some forms, the trap microfluidic channels 200, the transition from the flow microfluidic channels 160 to the trap microfluidic channels 200, or a combination of both the trap microfluidic channels 200 and the transition from the flow microfluidic channels 160 to the trap microfluidic channels 200 are configured to trap the micro-scale particles 500 in the trap microfluidic channels 200, whereby the trapped micro-scale particles 500, in combination, form an array 230 within the FTJ structure 140.

In some forms, the side walls 204 of the trap microfluidic channels 200 are straight and parallel to each other, where the height of the trap microfluidic channels 200 is less than the diameter of the micro-scale particles 500. In some forms, the height of the trap microfluidic channels 200 is more than the diameter or long dimension of the nano-scale objects 600.

In some forms, the side walls 204 of the trap microfluidic channels 200 are not straight such that the trap microfluidic channels 200 vary in width in a regular pattern along their lengths. In some forms, the pattern of width variation forms narrowings 208 in the width of the trap microfluidic channels 200, where the width of the narrowings 208 are less than the diameter of the micro-scale particles 500. In some forms, the width of the narrowings 208 are more than the diameter or long dimension of the nano-scale objects 600.

In some forms, all or a subset of the narrowings 208 overlap the flow layer 150 between some or each of the openings 166 on the bottoms of adjacent flow microfluidic channels 160. In some forms, all or a subset of the narrowings 208 overlap the opening 166 on the bottom of some or each of the flow microfluidic channels 160. In some forms, a subset of the narrowings 208 overlap the opening 166 on the bottom of some or each of the flow microfluidic channels 160 and a subset of the narrowings 208 overlap the flow layer 150 between some or each of the openings 166 on the bottoms of adjacent flow microfluidic channels 160. In some forms, a subset of the narrowings 208 overlap the opening 166 on the bottom of each of the flow microfluidic channels 160 and a subset of the narrowings 208 overlap the flow layer 150 between each of the openings 166 on the bottoms of adjacent flow microfluidic channels 160.

In some forms, the narrowings 208 overlapping the openings 166 on the bottoms of the flow microfluidic channels 160 form a small trap entrance 210 on the down-flow side 214 of the opening 206 and a large trap entrance 212 on the down-flow side 214 of the opening 206 for alternating trap microfluidic channels 200. In some forms, the size of the small trap entrance 210 is less than the diameter of the micro-scale particles 500. In some forms, the size of the small trap entrance 210 is more than the diameter or long dimension of the nano-scale objects 600. In some forms, the size of the large trap entrance 212 is more than the diameter of the micro-scale particles 500.

In some forms, the flow microfluidic channels 160 and the trap microfluidic channels 200 are at a right angle to each other. In some forms, the flow microfluidic channels 160 and the trap microfluidic channels 200 are at an oblique angle to each other. In some forms, the flow microfluidic channels 160 and the trap microfluidic channels 200 are at an angle of between 60° to 90°, between 60° to 90°, between 60° to 90°, between 60° to 90°, between 60° to 90°, between 70° to 90°, between 80° to 90°, between 85° to 90°, between 87° to 90°, between 88° to 90°, or between 89° to 90°, to each other.

In some forms, the side walls 204 of the trap microfluidic channels 200 are angled toward the up-flow ends 168 of the flow microfluidic channels 160.

In some forms, the microfluidic flow path 120 also includes a sample inlet 125. In some forms, the microfluidic flow path 120 is configured for movement of fluid 400 from the sample inlet 125 into the inlet conduit 130.

In some forms, the microfluidic flow path 120 also includes a plurality of outlet channels 290, wherein the microfluidic flow path 120 is configured for movement of fluid 400 from the trap microfluidic channels 200 into the outlet channels 290 and from the outlet channels 290 into the outlet conduit 300. In some forms, each trap microfluidic channel is flowably connected to a different one of the outlet channels 290.

In some forms, the flow layer 150 of the FTJ structure 140 also includes a plurality of outlet channels 290. In some forms, the outlet channels 290 are interspersed between and parallel to the flow microfluidic channels 160. In some forms, the outlet channels 290 each include a top 292, side walls 294, and an opening 296 on the bottom. In some forms, the outlet channels 290 and the trap microfluidic channels 200 allow fluid 400 movement from the trap microfluidic channels 200 into the outlet channels 290 via the opening 296 on the bottom and openings 206 on the top, respectively. In some forms, the microfluidic flow path 120 is configured for movement of fluid 400 from the trap microfluidic channels 200 into the outlet channels 290 and from the outlet channels 290 into the outlet conduit 300.

In some forms, the outlet channels 290 alternate with the flow microfluidic channels 160 in the flow layer 150 of the FTJ structure 140.

In some forms, the flow layer 150 of the FTJ structure 140 also includes an outlet channel 290. In some forms, the outlet channel 290 includes a top 292, side walls 294, and an opening 296 on the bottom. In some forms, the outlet channel 290 overlaps the down-flow ends 216 of the trap microfluidic channels 200. In some forms, the outlet channel 290 and the trap microfluidic channels 200 allow fluid 400 movement from the trap microfluidic channels 200 into the outlet channel 290 via the opening 296 on the bottom and openings 206 on the top, respectively. In some forms, the microfluidic flow path 120 is configured for movement of fluid 400 from the trap microfluidic channels 200 into the outlet channel 290 and from the outlet channel 290 into the outlet conduit 300.

In some forms, the chip is transparent in one or more regions. In some forms, the chip is transparent in a region corresponding to the array 230. In some forms, the array 230 includes a surface area of 25 mm² or less.

In some forms, the microfluidic platform 110 includes two microfluidic flow paths 120. In some forms, the two microfluidic flow paths 120 are flowably connected to a single fluid reservoir. In some forms, the fluid reservoir is flowably connected to the respective inlet conduits 130 of the two microfluidic flow paths 120.

In some forms, the two microfluidic flow paths 120 are symmetrically disposed on the microfluidic platform 110. In some forms, the two microfluidic flow paths 120 are symmetrically disposed on the chip.

In some forms, the microfluidic chip 100 also includes a plurality of micro-scale particles 500. In some forms, the micro-scale particle includes a microbead. In some forms, the microbead includes a magnetic microbead.

In some forms, the micro-scale particles 500 also include a first capture agent 710 specific for a target biomarker 910. In some forms, the first capture agent 710 is conjugated to the micro-scale particle via streptavidin. In some forms, one or more of the first capture agents 710 are bound to the target biomarker 910.

In some forms, the micro-scale particles 500 have a diameter of between about 1 μm and about 5 μm, inclusive. In some forms, each of the micro-scale particles 500 have between about 200,000 and about 400,000 first capture agents 710, inclusive.

In some forms, the first capture agent 710 is selected from the group consisting of an antibody, a nucleic acid, a protein, a lipid, a carbohydrate, and a small molecule. In some forms, the first capture agent 710 includes DNA or RNA, or both.

In some forms, the micro-scale particles 500 are located within the array 230. In some forms, the micro-scale particles 500 are located within the array 230 at a density of about 100 micro-scale particles/μm². In some forms, the array 230 includes from about 1×10⁴ micro-scale particles to about 1×10⁶ micro-scale particles, inclusive. In some forms, the array 230 includes about 4×10⁵ micro-scale particles. In some forms, the array 230 has an area of from about 10 mm² to about 50 mm², inclusive, optionally about 25 mm².

In some forms, the microfluidic chip 100 includes a plurality of nano-scale objects 600. In some forms, the nano-scale objects 600 include a second capture agent 720 specific for the target biomarker 910. In some forms, one of the second capture agents 720 are bound to one or more of the target biomarkers 910 bound to the first capture agents 710.

In some forms, the nano-scale objects 600 also include a reporter molecule 800. In some forms, the reporter molecule 800 includes a highly bright quantum dot nanoparticle 810.

In some forms, the micropillar structures 135 span the height of the inlet conduit 130. In some forms, the trap microfluidic channels 200 include a hydrophilic polymer. In some forms, the hydrophilic polymer includes polyethylene glycol (PEG).

Disclosed are methods for detecting a target biomarker 910 in a fluid sample 900. The method generally includes (a) introducing the fluid sample 900 to one or more of the microfluidic flow paths 120 of a microfluidic chip 100 as disclosed herein and (b) performing digital chromatography on the chip. In some forms, the fluid sample 900 includes, or is bought into contact with after its introduction, a plurality of micro-scale particles 500 and a plurality of nano-scale objects 600. In some forms, digital chromatography identifies the presence and/or quantity of the target biomarker 910 in the fluid sample 900.

In some forms, step (a) also includes introducing to a different microfluidic flow path 120 of the same chip a control sample including a known amount of the target biomarker 910.

In some forms, the performance of digital chromatography of step (b) includes actuating movement of fluid 400 through the microfluidic flow paths 120 in the microfluidic chip 100. In some forms, the movement filters and washes the micro-scale particles 500 within the FTJ structure 140. In some forms, the filtering of the micro-scale particles 500 in the FTJ structure 140 traps, and forms an array 230 of, the micro-scale particles 500 within the FTJ structure 140.

In some forms, step (b) also includes imaging the array 230 of micro-scale particles 500 within the microfluidic chip 100.

In some forms, the micro-scale particles 500 includes a microbead. In some forms, the microbead includes a magnetic microbead. In some forms, the micro-scale particles 500 also include a first capture agent 710 specific for a target biomarker 910. In some forms, the first capture agent 710 is conjugated to the micro-scale particle via streptavidin. In some forms, one or more of the first capture agents 710 are bound to the target biomarker 910.

In some forms, the nano-scale objects 600 include a second capture agent 720 specific for the target biomarker 910. In some forms, one of the second capture agents 720 is bound to one or more of the target biomarkers 910 bound to the first capture agents 710.

In some forms, the nano-scale objects 600 also include a reporter molecule 800. In some forms, the reporter molecule 800 includes a highly bright quantum dot nanoparticle 810.

In some forms, step (b) also includes detecting and measuring the target biomarkers 910 bound to the first capture agents 710 on the micro-scale particles 500 within the array 230.

In some forms, the method also includes, prior to step (a), (i) incubating the fluid sample 900 with the micro-scale particles 500 for a time and in an amount effective for binding of the target biomarkers 910 to the first capture agent 710. In some forms, following step (i), washing the micro-scale particles 500.

In some forms, the method also includes, prior to step (a), contacting the micro-scale particles 500 with the nano-scale objects 600 for a time and in an amount effective for binding of the target biomarkers 910 to the second capture agent 720.

In some forms, steps (a) and/or (b) include a total time of between about 10 and 1000 about seconds, inclusive, optionally about 180 seconds.

In some forms, the method detects at least 90%, such as 91%, 92%, 93%, 94%, 95%, 96%, 97%, 98%, 99%, or 100% of the biomarkers within the fluid sample 900. In some forms, the fluid sample 900 includes a biomarker at a concentration of about 1 IU/mL. In some forms, the fluid sample 900 includes a biomarker at a concentration of about 10⁻¹⁸ to 10⁻⁵ M. In some forms, the fluid sample 900 includes a volume of between about 1.0 μL to about 100 μL, inclusive, optionally about 50 μL.

In some forms, the fluid sample 900 includes between one and 100 different biomarkers, optionally between one and 10 different species of biomarkers. In some forms, one biomarker is an immunoglobulin. In some forms, one biomarker is an IgE. In some forms, one biomarker is an interferon. In some forms, one biomarker is TNF-alpha.

In some forms, the target biomarker 910 within the fluid sample 900 is derived from a bodily fluid from a subject. In some forms, the bodily fluid is selected from the group including blood, sweat, semen, serum, bile, saliva, tear fluid, pus, mucus, pleural fluid, vitreous fluid, spinal fluid, synovial fluid, amniotic fluid, and urine. In some forms, the bodily fluid is tear fluid.

Component Reference Numbers

| Reference | Component | Part of |
|---|---|---|
| 100 | microfluidic chip | — |
| 110 | microfluidic platform | microfluidic chip |
| 120 | microfluidic flow path | microfluidic platform |
| 125 | sample inlet | microfluidic flow path |
| 130 | inlet conduit | microfluidic flow path |
| 135 | micropillar structure | inlet conduit |
| 140 | flow-trap junction (FTJ) structure | microfluidic flow path |
| 150 | flow layer | flow-trap junction (FTJ) structure |
| 160 | flow microfluidic channel | flow layer (of FTJ structure) |
| 162 | top | flow microfluidic channel |
| 164 | side walls | flow microfluidic channel |
| 166 | opening | flow microfluidic channel |
| 168 | up-flow end | flow microfluidic channel |
| 190 | trap layer | flow-trap junction (FTJ) structure |
| 200 | trap microfluidic channel | trap layer (of FTJ structure) |
| 202 | bottom | trap microfluidic channel |
| 204 | side walls | trap microfluidic channel |
| 206 | opening | trap microfluidic channel |
| 208 | narrowing | trap microfluidic channel |
| 210 | small trap entrance | trap microfluidic channel |
| 212 | large trap entrance | trap microfluidic channel |
| 214 | down-flow side | trap microfluidic channel |
| 216 | down-flow end | trap microfluidic channel |
| 230 | array | flow-trap junction (FTJ) structure |
| 290 | outlet channel | microfluidic flow path |
| 292 | top | outlet channel |
| 294 | side walls | outlet channel |
| 296 | opening | outlet channel |
| 300 | outlet conduit | microfluidic flow path |
| 400 | fluid | — |
| 500 | micro-scale particles | — |
| 600 | nano-scale objects | — |
| 700 | capture agent | micro-scale particles; nano-scale objects |
| 710 | first capture agent | micro-scale particles |
| 720 | second capture agent | nano-scale objects |
| 800 | reporter molecule | — |
| 810 | quantum dot nanoparticle | reporter molecule |
| 900 | fluid sample | — |
| 910 | target biomarker | fluid sample |

### II. Systems for Digital Immunochromatography

Systems and methods for detection and quantitation of a target biomarker within a sample in vitro using digital immunochromatography have been developed.

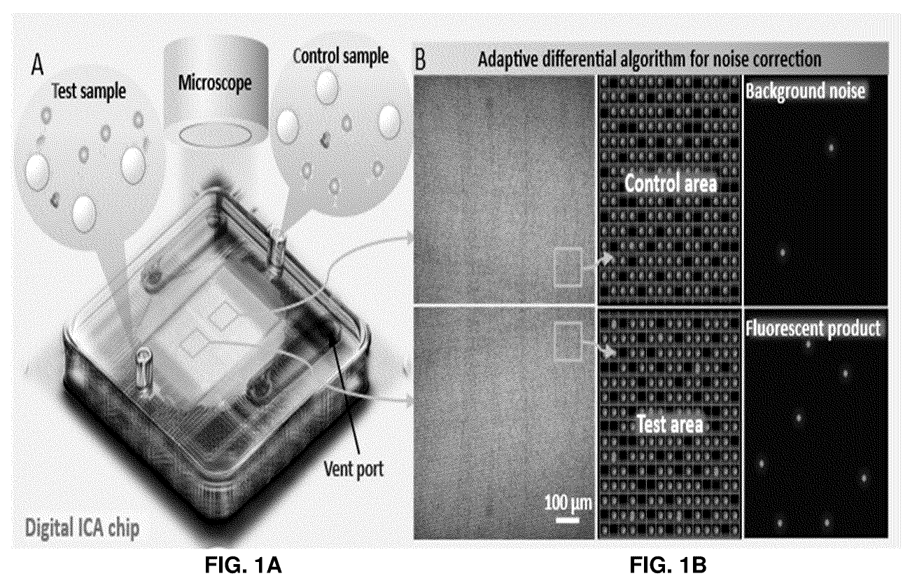

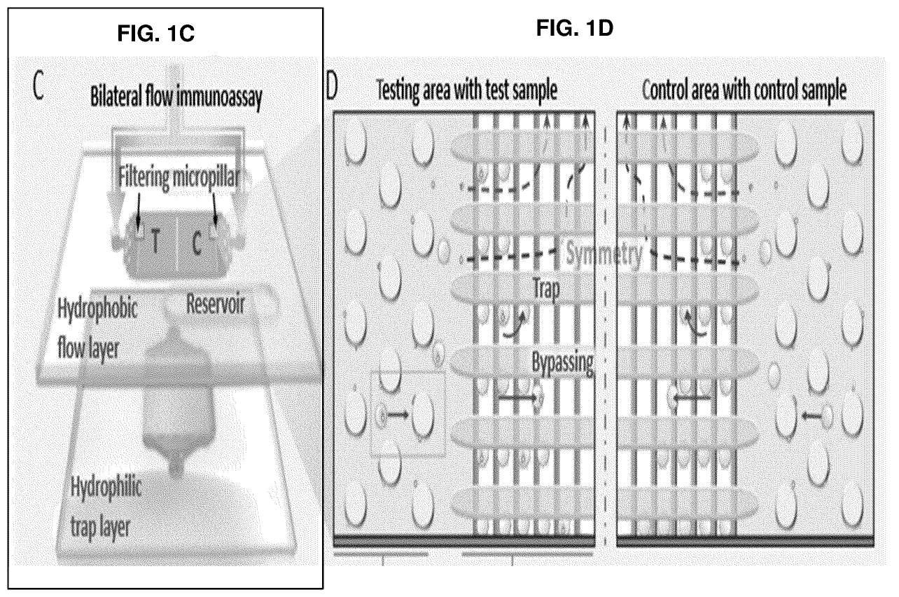

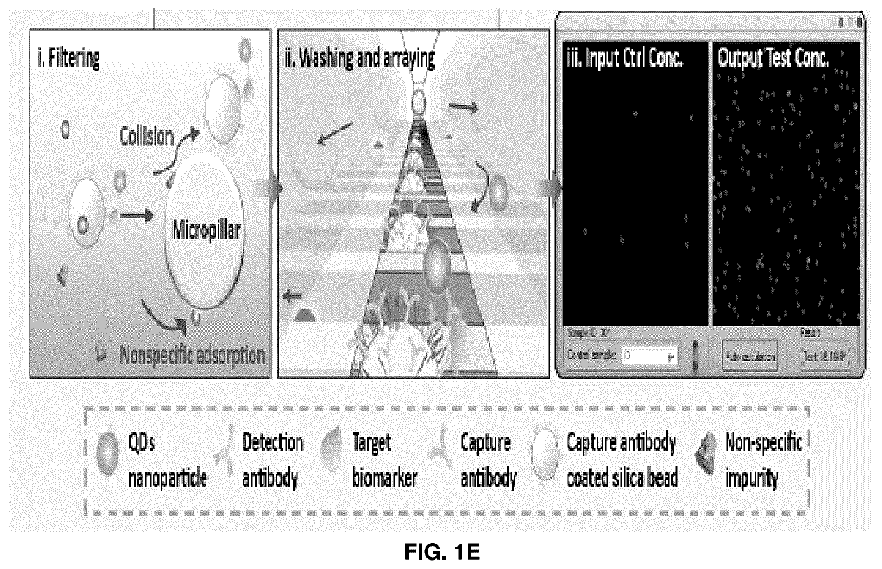

To overcome quantitative limitations of conventional lateral flow immunoassays (LFIAs) without sacrificing their advantages, a silica bead-based Bilateral Zigzag Flow Immunoassay (BiZi-FIA) with an Adaptive Differential Noise Correction (ADNC) algorithm has been developed as a platform that merges the ease-of-use of lateral flow with the precision of digital immunoassay techniques. An overview of an exemplary assay is set forth in FIGS. 1A-1B).

The described systems for BiZi-FIA leverage a symmetrical bilateral assay configuration, wherein test and control samples are introduced from opposing sides of a microfluidic channel, induced bilateral zigzag flow gets through the flow and trap channel formed grids array (FT-JA). This dual-layer FT-JA generates high-density bead arrays (e.g., 9.7×10³ beads/mm²) through hydrodynamic focusing, achieving approximately 93%, or more, capture efficiency in 180 seconds or less. In some forms, the BiZi-FIA captures beads at an amount greater than conventional single molecular arrays, for example, 1.1× to 10× denser, such as 5× denser.

The described design employs symmetrical environmental control to ensure consistent sample exposure, thereby enabling real-time concentration determination through Adaptive Dynamic Noise Correction (ADNC). As an advanced machine learning framework, ADNC revolutionizes analytical workflows by eliminating traditional calibration curve requirements through differential signal analysis of test/control channels. The algorithm dynamically refines analytical parameters using gradient descent optimization, iteratively enhancing model accuracy through cumulative experimental data. This iterative process establishes a robust correlation between predicted and measured values, precisely resolves signal gradients across 6 orders of magnitude (0.01-10,000 fM) with attomolar sensitivity.

Collectively, these integrated innovations establish an effective digital immunochromatographic platform, rigorously validated through multiplexed detection of three clinically significant biomarkers, immunoglobulin E (IgE), tumor necrosis factor-alpha (TNF-α), and neurofilament light chain (NFL), in both controlled laboratory settings and human tear matrices. The system demonstrated exceptional sensitivity with limits of detection (LOD, 3σ criterion) of 13 aM (0.013 fM) for IgE, 50 aM for TNF-α, and 810 aM for NFL, representing 2-3 order-of-magnitude improvements over conventional lateral flow assays. The complete analytical workflow requires only 40 minutes total processing time: 30 minutes for incubation (target capture/antibody conjugation), followed by <10 minutes for automated bead array imaging and signal quantification, an 80% reduction compared to standard digital ELISA protocols. The accuracy of BiZi-FIA was rigorously validated through spike-and-recovery experiments. For all three biomarkers (IgE, TNF-α, NFL), the measured concentrations showed quantitative recovery rates of 92-107% across clinically relevant ranges. This demonstrates the robustness against matrix effects in complex biological fluids. Critical clinical validation using minimally processed human tear samples (2.2 μL volume, 1:32 PBS dilution) confirmed the combine diagnostic capability with broaden concentration range of multiple biomarkers (n=3 replicates per sample). The attomolar sensitivity range particularly positions this technology for non-invasive monitoring of chronic inflammatory conditions (via TNF-α/IgE) and neurodegenerative disorders (via NFL), with immediate applications in early-stage dry eye disease diagnosis, real-time tracking of allergic response dynamics and point-of-care neurotrauma assessment.

This performance spectrum, combining microsample requirements, rapid processing, and molecular-level sensitivity, addresses critical gaps in current diagnostic paradigms. By enabling quantitative multiplexed analysis from non-invasive specimens, the platform establishes a new framework for precision medicine implementation in resource-limited settings.

### A. Compositions for Digital Immunochromatography (BiZi-FIA)

Compositions for BiZi-FIA including microfluidic platforms for actuating the detection of one or more target molecules within a fluidic sample by digital immunochromatography are described. Typically, the compositions include a microfluidic chip including a microfluidic channel and a filtration system, that excludes contaminants on the basis of size and charge. In some forms, the BiZi-FIA systems utilize luminescent quantum dot nanoparticles as biomarker labels. In some forms, the BiZi-FIA systems utilize gold or other metal nanoparticles to amplify detection signals. In some forms, the BiZi-FIA systems incorporate a double-layer flow and trap (FT) junction array that functions as a molecular sieve. For example, in some forms, the BiZi-FIA systems include one or more filters deposed within the microfluidic channel are sized to selectively retain microbeads carried with target biomarkers and expel particles at the nanometer scale to increase capture efficiency and to selectively minimize non-specific signal interference.

The described compositions and methods for BiZi-FIA achieve a facile beads-based accurate analyte assay to improve the capture efficiency of target molecular biomarkers and minimize non-specific signal interference. As described in the methods, implementation of the BiZi-FIA with a high-density FT junction array has demonstrated a substantial increase in microbead capture efficiency—from 40% to 90%, enabling 4×10⁵ of beads arraying on a 2D plane (30 mm²), which facilitates swift imaging and accurate analysis for a comprehensive range of target biomarkers within a sample with a field of view diameter 6.25 mm (Nikon ECLIPSE Ti2, 20 mm FOV).

The design of the chip allows both microbead-based antibody capture and direct modification on the chip surface, demonstrating versatility in application. The design of the chip offers superior dispersion capabilities for the analytes and the ability to control single variables during testing. This versatility and control are unique in the context of immunoassays.

The chip's design allows for stringent control of a single variable during the assay. It differentiates effective signals from background noise based on control area readings. If the background noise exceeds the error range of the standard curve, the test is considered invalid; otherwise, the effective signal is calculated by subtracting the background noise from the test signal. This method of signal differentiation and control is both innovative and non-obvious.

The chip's design allows for aspects of this chip include integration of surface chemistries, structural design for filtration and washing, dual-layer architecture for particle control, selective surface modification, and versatile application in immunoassay technology with enhanced signal control and differentiation. These features collectively contribute to the described systems and methods' distinction from existing technologies.

BiZi-FIA platforms, such as chips, designed according to the described requirements of BiZi-FIA methods for detection and measurement of biomarkers within a biological sample are described. Mobile phase matrices, such as microparticles, for use in the devices are also described. Target biomarkers and capture agents for specific binding to the target biomarkers are also described. Reporter molecules, such as nanoscale fluorescent markers or labels for detection are also described.

### (i) BiZi-FIA Platform Design

In some forms, systems for BiZi-FIA are implemented within a self-contained unit, such as a plate, biochip, or chip, or array. In an exemplary form, the systems for BiZi-FIA are implemented within a single chip. A chip typically includes a platform for forming an array of microbeads, including:
(i) one or more microfluidic channel for the channeling of fluids within and across the chip;
(ii) a flow-trap junction filtration structure (including a size-exclusion (trap) filtration system, and a hydrophilic (flow) filtration system; and a hydrophobic filtration system;
whereby the flow-trap junction filtration structure is implemented within the microfluidic channels, and whereby the trap collects microparticles for visualization.

In some forms, the BiZi-FIA includes a composition of two or more different layers, for example an upper and a lower layer that are combined to form a defined microfluidic channel.

Typically, at least a portion of the BiZi-FIA includes a region that permits inspection, for example, by a light microscope. Typically, the portion of the BiZi-FIA that includes the trap region will create the concentrated microparticle array that is to be imaged, for example, by a light microscope.

In some forms, all or part of at least one layer or section of a BiZi-FIA device is formed of or includes a polymer, such as an inert polymer. In some forms, all or part of a BiZi-FIA device is formed of or includes a metal. In some forms, all or part of a BiZi-FIA device is formed of or includes glass. In some forms, all or part of a BiZi-FIA device is formed of or includes an opaque structure that does not permit passage of light through the structure. In some forms, the BiZi-FIA device is formed of or includes transparent material, for example that permits passage of one or more different wavelengths of light through the device.

Typically, the BiZi-FIA device is configured to impart uniform distribution and/or flow of particles through the microfluidic channels.

### (a) Microfluidic Channels

An exemplary chip is configured to include one or more microfluidic channels for the channeling of fluids across the chip. Typically, each chip includes at least two microfluidic channels. Each microfluidic channel includes an inlet and an outlet. Typically, the microfluidic channel connects the inlet to the outlet. In some forms, the microfluidic channel includes one or more mid-sections located between the inlet and the outlet. Typically, the inlet of the microfluidic channel is connected to one or more fluid reservoirs. An exemplary fluid reservoir includes a wash buffer.

In some forms, each microfluidic channel of a chip having a plurality of microfluidic channels includes an inlet, whereby the inlet is open for addition of a sample into the microfluidic chamber. Therefore, in some forms an inlet includes an aperture sized to accommodate a pipette tip.

In some forms, the chip design includes one or more first microfluidic channels for conducting the passage of a testing sample, and one or more second microfluidic channels for conducting the passage of a control sample. An exemplary control sample is a control sample including a known amount and/or concentration of at least one known biomarkers. In some forms, the one or more first microfluidic channels and the one or more second microfluidic channels share the same washing buffer inlet on either side. For example, in some forms, the chip design includes one or more “testing” microfluidic channels for conducting the passage of a sample and one or more “control” microfluidic channels for conducting the passage of a control sample arranged in a symmetric pattern on either side of the same chip. In some forms, the symmetry in structure facilitates simultaneous sample introduction and/or processing in both microfluidic channels, allowing for integrated control and comparative analysis. In some forms, the structure of the chip is configured to facilitate simultaneous quantitation of the control and test samples in two structurally analogous microfluidic chambers, positioned opposite one another on the chip in a symmetrical configuration. In some forms, the chip is configured such that the flow and distribution of microparticles within analogous the test and control microfluidic chambers of a chip is equivalent.

### (b) Bilateral Zigzag Flow Directed High-Density Microbeads Trap (Flow/Trap Junction Array)

In an exemplary chip, a microfluidic channel incorporates a flow-trap junction (FTJ) for the filtration and washing of solid-phase particles within the BiZi-FIA device.

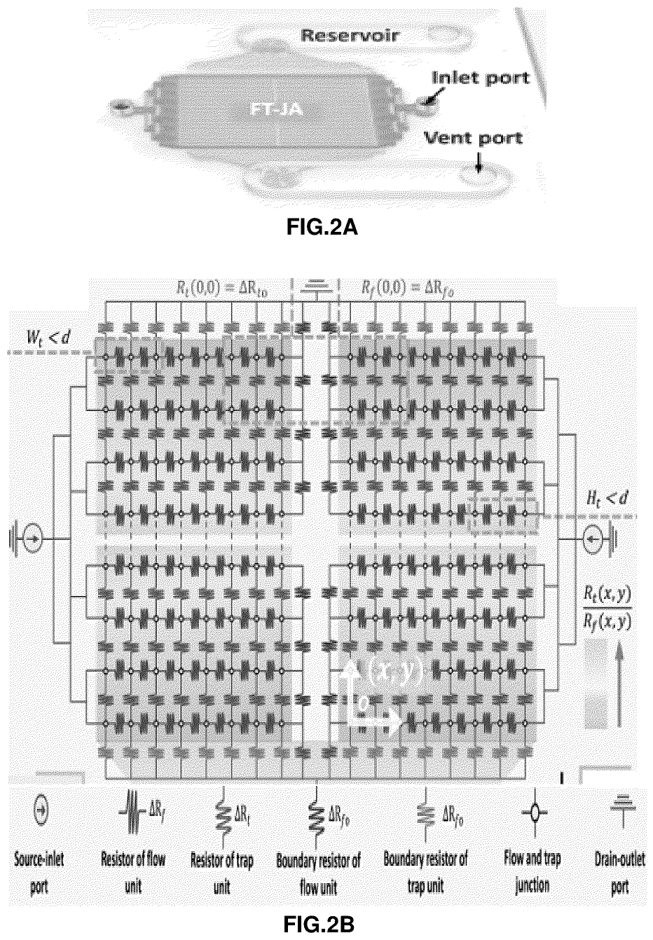

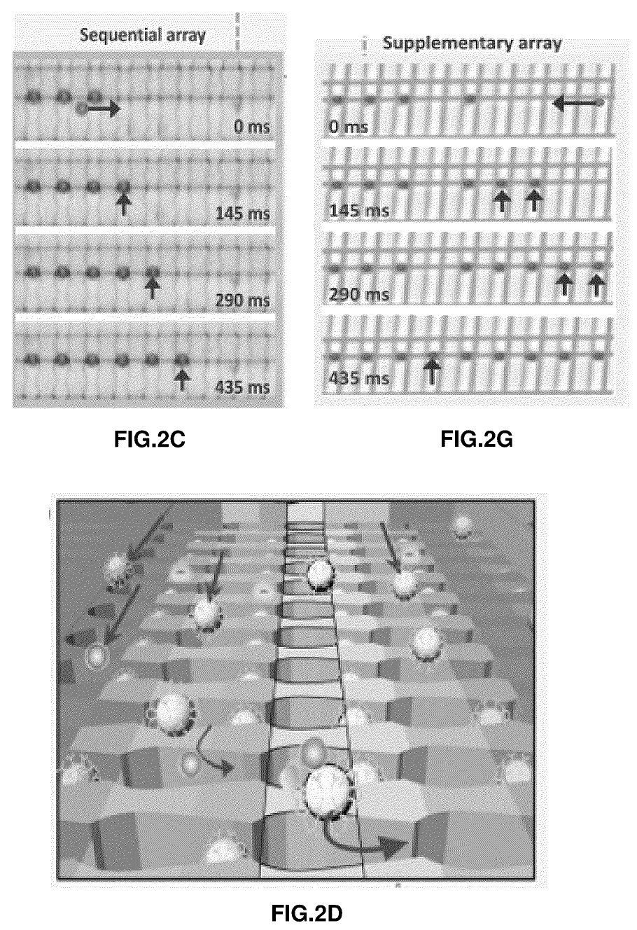

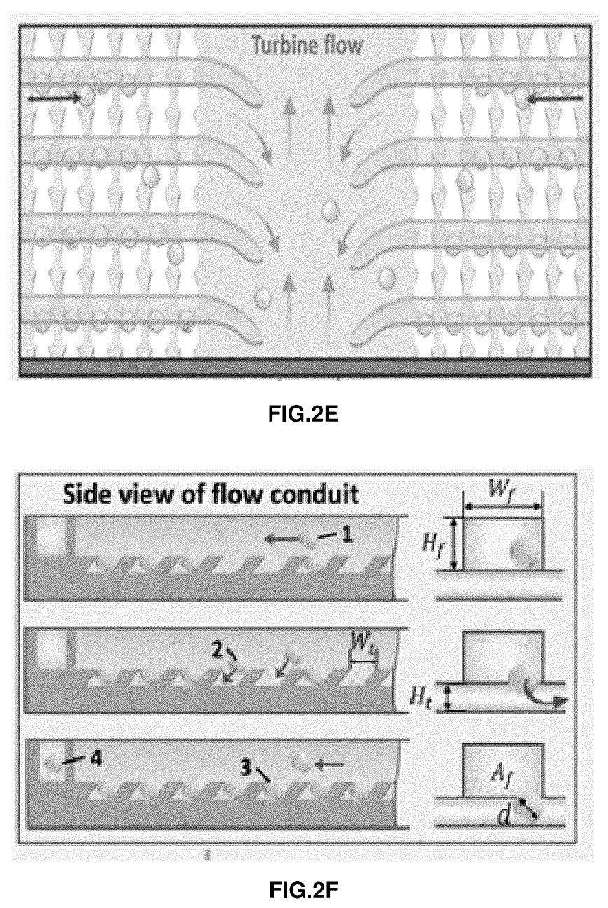

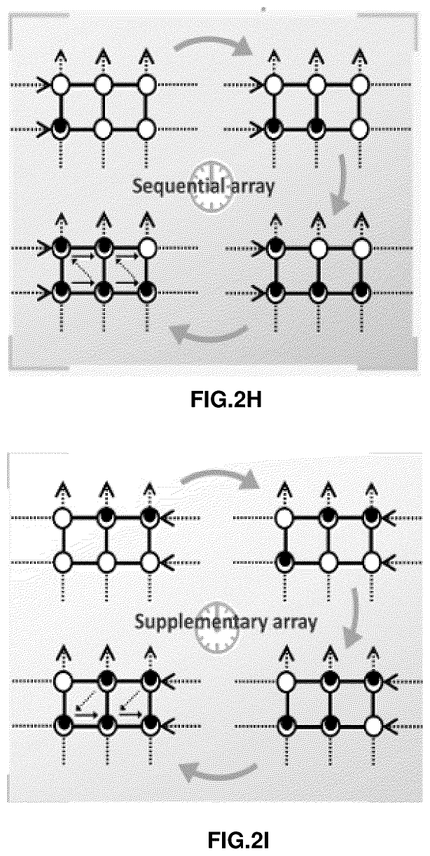

In single-molecule protein detection, the capturing efficiency of microbeads within a limited detection area is critical due to the low concentration of target proteins. Effective microbead capture is essential to enhance measurement reliability and maintain assay sensitivity. Inadequate capture can lead to significant errors, such as the underestimation of protein concentrations and false negatives. To address this challenge, the BiZi-FIA utilizes FT-JA functions as a refined molecular sieve, characterized by overlapping flow and trap layers that separate and localize target molecules within a confined space (FIG. 2A). Each flow channel connects to the two sides of the inlet conduit with a branched structure in the upper layer, while each trap channel links to the two sides of outlets with a reservoir in the lower layer (FIG. 2B). The crisscrossed FT junctions, composed of interlaced straight flow and trap channels, create narrowed pocket regions that serve as primary traps for microbeads, forming a zigzag flow path for the microfluidic system. In terms of microbead loading, when a trap is vacant, it offers lower flow resistance compared to the flow direction, causing microbeads to enter the trap channel, and be captured (FIG. 2C). Once inside, the captured microbeads act as a c, dramatically increasing the flow resistance within the trap, and therefore redirecting the main flow current back into the flow channel. Subsequent microbeads are directed along the flow channel, bypassing occupied traps and seeking the next available trap with a lower-pressure region (FIG. 2D).

The primary objective of the FTJ is to capture micro-scale solid phase particles (i.e., microbeads) for efficient washing and filtration. Typically, the FTJ is configured to uniformly distribute biomarkers, which adhere to the Poisson distribution, within a confined area. Typically, the arrangement of the FTJ facilitates efficient separation and precise spatial localization of target biomarkers for ultrabright fluorescent labelling.

The Flow and Trap Junction Array (FTJA) is a high-density filter system characterized by its overlapping flow and trap layers.

Hydrodynamic forces in a flow and trap junction array (FTJA) allow simultaneous transportation and immobilization of a large number of particles. The primary objective of the FTJA is to facilitate the smooth and efficient guiding of microspheres into capture traps by leveraging a slightly inclined branch. Capturing and immobilizing microbeads in the FTJA is achieved by introducing forces opposing their flow direction, resulting in localized regions of increased flow resistance.

The filter-like Flow and Trap Junction concept entails the strategic arrangement of flow and trap conduits to create a directed flow pattern that guides microspheres toward efficient capture traps. Inspired by the asymmetrical behavior of filter devices, the FTJA exhibits a favorable flow pattern, allowing easy ingress of microspheres into the trap regions while minimizing backflow, thus enhancing trapping efficiency.

To facilitate the smooth and facile guidance of particles towards the capture traps, a tilted supportive flow and trap structure was developed, rather than perpendicular to the main flow direction. This design criterion ensures that microspheres encounter reduced resistance during transit, promoting smooth movement toward the capture traps. The inclined support flow direction provides a balanced compromise between minimizing resistance and maximizing trapping efficiency, contributing to successful microsphere immobilization.

By arranging flow and trap conduits at different horizontal levels and establishing vertical connections, the FTJA enables parallel multi-conduit operation. This configuration allows for the simultaneous capture of multiple microspheres and enhances the overall capture capacity, crucial for various applications requiring large-scale microsphere manipulation. By aligning the trap direction with the supportive flow direction, trapping efficiency was optimized while minimizing resistance faced by the microspheres. This design ensures that the particles experience minimal energy loss during the capture process, leading to more efficient and stable immobilization within the traps.

The described High-Density Filter-like Flow and Trap Junction offer several key advantages. Firstly, the design enhances trapping efficiency, ensuring a high capture rate of microspheres passing through the device. Secondly, the lowered fluid resistance provides for smoother particle guidance, reducing the likelihood of particle escape or clogging. Furthermore, the junction's filter-like behavior promotes unidirectional flow, minimizing backflow and improving the overall system's reliability and repeatability. To achieve high throughput and accommodate a large number of particles, a high-density and parallel multi-conduit was designed.

This involved layering flow capture conduits at different horizontal planes and establishing vertical connections. As a result, the device can simultaneously capture multiple microspheres, ensuring efficient use of the available capture area and enhancing the overall particle capture capacity.

In some forms, the FT junction array includes two distinct layers: one “flow” layer a hydrophobic PDMS surface and one “trap” layer with PEG-coated surface.

Typically, the upper flow layer includes numerous parallel flow conduits, while the lower trap layer is made up of an array of parallel trap conduits. In some forms, these two layers combine together to create a flow and trap junction array.

Each flow and trap junction is crafted from a straight flow conduit paired with a pocket-shaped trap conduit. These conduits intertwine to form a single FT junction. Typically, the narrowed pocket regions beneath the straight flow conduit serve as the primary traps.

Typically, each flow conduit is connected to an inlet conduit with a branched structure, while every trap conduit directly links to an outlet. When a trap is vacant, the trap conduit offers a lower flow resistance compared to the flow conduit. Once inside, the microbead acts as a barrier, dramatically increasing the flow resistance within the trap conduit. This forces the main current back into the flow conduit. Any subsequent microbeads are then directed along the flow conduit, effectively bypassing any occupied traps, and actively seeking the next available trap.

In some forms, an outlet conduit of the device includes one or more turbine valve(s) configured to provide a directional fluid flow within the outlet conduit. These valves are termed “Tesla-style” turbine valves. In some forms, each flow microfluidic channel includes a terminus region proximal to the outlet conduit that is configured to include a curvature at the terminus of the flow microfluidic channel. Typically, the curvature is less than 90 degrees and is uniform among all microfluidic channels within the device. The curvature is generally sufficient to direct the flow of fluid out of the FTJ in a direction that is opposing that of a directional fluid flow within the outlet conduit. Therefore, in some forms, the opposing flow directionality provides a resistance force within the flow microfluidic channels. Typically, the resistance force is in an amount effective to direct fluid movement laterally through the trap microfluidic channels within the FTJ. The opposing flow created by the turbine valve/curved configuration of flow channels is depicted in FIG. 2E.

### (1) FTJ Design Parameters

The concept and design criterion of the High-Density Filter-like Flow and Trap Junction generally provide a solution for efficient microsphere capture and immobilization within microfluidic devices.

By leveraging the isotropic nature of spherical microbeads and strategically arranging flow and trap conduits, the FT junction array ensures high-efficiency microsphere capture, thereby presenting a tool for diverse applications, including biome diagnostics, cell sorting, and drug delivery systems.

In some forms, the FTJ includes a plurality of overlapping hydrophobic and hydrophilic interfaces, for example, amenable to enhance filtration and washing of microparticles within the chip. The structure of the filtration system also includes a “trap” that excludes passage of microparticles at a single site but permits passage of nanoscale particles. The trap structure concentrates the microparticles within a total area sized for imaging of the concentrated microparticles in a single frame of a microscope.

To ensure effective microbead capture by the bilateral zigzag flow, the resistance in the trap direction (Rt) should be smaller than in the flow direction (Rf). By calculating and distributing these resistance ratios, the design of flow and trap channels can be guided to optimize capture efficiency and flow performance. The resistance in the flow direction is defined as R_{f}(x, y) and in the trap direction as Rₜ(x, y), as illustrated in FIG. 2B. As the boundary conditions of FT-JA coordinate system, the origin (0,0) represents the final FT junction closest to the outlet, with the minimum resistances denoted as R_{f}(0,0)=ΔR_{fo} and Rₜ(0,0)=ΔRₜₒ, which values are influenced by the geometry of flow and trap unit respectively [23, 24]. Considering the parallel relationship between flow and trap direction at each junction, the resistance at one junction point, Rⱼ(x, y), is articulated as:

$${R}_{j} ( x , y ) = \frac{{R}_{t} ( x , y ) * {R}_{f} ( x , y )}{{R}_{t} ( x , y ) + {R}_{f} ( x , y )} \qquad [ 1 ]$$

For positions where x≥1 and y≥0, the resistance in the flow direction, R_{f}(x, y), is determined by the resistance at the preceding junction along x-axis (flow direction), Rⱼ(x−1, y), and the resistance of the current flow unit, ΔR_{f}(x,y), which is determined by the geometry of a flow unit at (x,y):

$${R}_{f} ( x , y ) = Δ {R}_{f} ( x , y ) + \frac{{R}_{t} ( x - 1 , y ) * {R}_{f} ( x - 1 , y )}{{R}_{t} ( x - 1 , y ) + {R}_{f} ( x - 1 , y )} \qquad [ 2 ]$$

Similarly, for x≥0 and y≥1, the resistance in the trap direction, Rₜ (x, y), is determined by the preceding junction along the y-axis (trap direction), Rⱼ (x, y−1), and the resistance of current trap unit, ΔRₜ (x,y), which is determined by the geometry of a trap unit at (x,y):

$${R}_{c} ( x , y ) = Δ {R}_{t} ( x , y ) + \frac{{R}_{t} ( x , y - 1 ) * {R}_{f} ( x , y - 1 )}{{R}_{t} ( x , y - 1 ) + {R}_{f} ( x , y - 1 )} \qquad [ 3 ]$$

Based on these recursive relationships, the capture efficiency of the FT-JA in the BiZi-FIA system can be predicted by the resistance ratio between the trap and flow direction, Rₜ (x, y)/R_{f} (x, y). When Rₜ (x, y)/R_{f} (x, y)<1, the corresponding junction works effectively to capture the microbeads. The total resistance, Rₜₒₜₐₗ, of FT-JA is calculated via equivalent circuit simulation (FIG. 2B).

$${R}_{total} = {( {\sum }_{i = 1}^{N} \frac{1}{{R}_{j} ( {x}_{ma} )} )}^{- 1} \qquad [ 4 ]$$

Simulation results (Table 1) indicated that maintaining a higher number of trap channels relative to flow channels reduces, Rₜₒₜₐₗ, favoring microbead capture by minimizing resistance in the trap direction. Specifically, scenarios where ΔRₜ (x, y)<ΔR_{f} (x, y), achieve maximum capture efficiency. To facilitate this, we designed inclined trap channels that guide flow lines preferentially into the trap layer. In contrast, vertical trap channels increase resistance and hinder capture efficiency. Consequently, the inclined trap channel design optimizes capture efficiency within the BiZi-FIA system (FIG. 2F).

In some forms, for laminar flow in microchannels, the pressure drop is analysed using the Hagen-Poiseuille equation for cylindrical conduits, which denotes the pressure drop due to viscous flow in a cylindrical pipe. The Darcy friction factor, f, is related to the aspect ratio, a, and Reynolds number, Re=μVD/μ, where μ is the fluid viscosity, ρ is the fluid density, V is the average velocity of the fluid, and D is the hydraulic diameter, respectively. The aspect ratio α is defined as the ratio of the smaller dimension (height H) to the larger dimension (width W) of the channel, α=H/W, such that 0≤α≤1. The product of the Darcy friction factor f and the Reynolds number Re is a constant that depends on the aspect ratio, i.e., f·Re=C(α), where C(α) denotes a constant that is a function of α. After simplifications, the expression obtained is:

$$Δ {R}_{f} ( x , y ) = C ( α ) \cdot \frac{μ {{L}_{f} ( {W}_{f} + {H}_{f} )}^{2}}{8 {W}_{f}^{3} {H}_{f}^{3}} ,$$

where L is the length of the channel.

In an exemplary form, the FTJ includes a hydrophobic and a hydrophilic layer, each layer full of isometric structures. Typically, the two layers of structures are arranged vertically, forming a high-density FT junction array. The FT junction array allows high-resolution recognition of target biomarkers with ultrabright fluorescent nanoscale labels (nanoparticles of quantum dots). The FT junction array provides high resolution, high-efficiency, and high-throughput single-molecule array.

Typically, the oblique trap junction optimizes injection performance of mechanical pumps and pipettes under low flow resistance conditions. For example in some forms, during operation, the particle suspension enters the side conduit on the trap layer from the main conduit on the flow layer. Due to the presence of an inclined plane, only particles with a diameter smaller than the height of the inclined plane can pass through the side conduit, while particles with a diameter larger than the height of the inclined plane will be blocked at the entrance of the side conduit and eventually concentrated in the main conduit. This structure achieves precise sorting and centralized control of high-density particle arrays. When the flow direction is biased, the original array of particles can be collected again. Therefore, the microbeads are repeatedly arrayed, cleaned, and resuspended in the chip with low flow resistance.

Typically, the entire region encompassing the flow-trap junction structure is sized to be compliant with maximum objective field of view of a bright-field microscope, for example of about 30 mm², to capture an image including, for example, two or more microbead arrays. Typically the concentrated microbeads within the trap structure of a single array occupy a total area of about 2.5×2.5 mm. For example, in some forms, for bright-field microscopy, an array occupies an area of about 2.5×2.5 mm, for imaging within a single frame. Typically, images are obtained in two conduits to identify the “on” beads, and the “off” beads.

### (2) Drainage Channels/Outlets

The described FTJ includes a plurality of drainage conduits drainage conduits for passage of fluids through the microfluidic device. Typically, the conduits overlap with access conduits, forming rows of grid-like array chambers. Therefore, in some forms, the structure forms a filter-like beads separator system. When liquids are displaced using typical hydrodynamic confinement in this system, the device provides a convenient and high-efficiency platform to transport and immobilize microbeads, infuse reagents, and arrange the microbeads in the desired array manner for many bioanalytical applications.

In some forms, the chip structure introduces a design optimized to efficiently eliminate non-specific molecular impurities. This is achieved by featuring interlocking, comb-like flow conduits and drainage conduits. These conduits are arranged in an overlapping and interlacing pattern on the same plane, creating an array of chambers in a grid-like formation within the trap channel. This intricate layout optimizes separation and removal of undesired molecules, enhancing the precision and effectiveness of the immunoassay process.

The designed unidirectional microcapillary flow allows simultaneous transportation and immobilization of many microbeads in a chip. Compared with single-conduit perfusion with negative injection pressure, parallel perfusion of multiple conduits is more efficient. Typically, a drainage conduit is parallel to the extending direction of an array conduit. That is, the guiding conduit and the extending direction of the capturing conduit are consistent. Typically, the array chamber captures the particles in the liquid, and the excess liquid flows into the drainage conduit.

### Drainage Chamber

In some forms, the drainage conduit includes a drainage chamber, which is a liquid storage tank, e.g., located on the left and/or right sides, configured to store the liquid after passing through the array chamber.

In some forms, the drainage conduit has a row of 3D re-entrant cavities. When the cross-section of cavity is semi-elliptical and inclined, the shearing force is more likely to shear the liquid, and the liquid enters the cavity. The bubbles and dead zones in the inclined cavity are less likely to be generated than in the vertical cavity. Typically an oblique conduit can better reduce the pinning effect than the vertical conduit, enabling faster liquid transport. In some forms, an oblique conduit provides the ability to utilize the three-dimensional surface energy gradient and Laplace pressure difference to achieve the spontaneous flow of liquid so that the liquid fills the reservoir under the capillary force of the structure of bionic Nepenthes peristome without the need for a complicated mechanical injection pump device, which has the characteristics of simple operation and high repeatability.

Upon entering the intermediate array region, the single-molecule immunocomplexes captured by the magnetic beads are subjected to the contrasting properties of the array region's surface. The hydrophilic and anti-static adsorption properties of this region, which are opposite to the hydrophobic properties of the flow separation zone, effectively prevent the non-specific adsorption of quantum dot microspheres in the array area. As a result, they continue to be expelled out of the chip with fluid dynamics. The chip, specifically designed for this purpose, efficiently removed the rest of non-specific impurities in its flow layer's separation zone. The chip's design, which leverages the hydrophobic nature of Polydimethylsiloxane (PDMS), ensures the effective removal of non-specifically bound proteins and other impurities. This design significantly improves the signal-to-noise ratio and sensitivity of the detection.

A large fraction of seeded quantum dots nanosphere adhered to the microwell surface due to a combination of surface interactions (electrostatic, van der Waals, steric, hydrophobic, and hydration forces) and therefore showed no Brownian motion.

### (3) Hydrophobic Interfaces

In an exemplary chip, a microfluidic channel incorporates a plurality of hydrophobic interfaces. The hydrophobic interface typically includes a filtering fractal structure including a plurality of micropillars; in operation, particles (such as microparticles) navigate through the filtering fractal structure, which facilitates the constant collision of microbeads with the micropillars, leading to the dissociation of non-specifically adsorbed nanoparticles.

In some forms, a plurality of hydrophobic (e.g., superhydrophobic) structures are present in the form of micropillar structures. Exemplary micropillars span all or part pf the distance between the top and the bottom of a microfluidic channel. Therefore, in some forms, a micropillar of a plurality of micropillars includes a structure that connects the top and bottom of a microfluidic channel. In other forms, a micropillar spans at least 50% of the distance between the top and the bottom of a microfluidic channel. In some forms, the size and shape and position of a micropillar is suitable to impede and/or alter the passage of a microparticle within the microfluidic channel, but not to prevent passage of a microparticle. In some forms, the shape of the micropillar is rounded, such that a microparticle that contact the micropillar will glance off the micropillar such that the trajectory of the microparticle is altered following contact with the micropillar. Therefore, in some forms, a plurality of micropillars are situated in a manner that directs the movement of microparticles flowing within the microfluidic channel.

Typically, the micropillars are formed from a hydrophobic material and have a hydrophobic fluid characteristic. In some forms, the micropillars include hydrophobic surface that imparts non-specific adsorption properties. Therefore, in some forms, the micropillars include a hydrophobic surface to capture and filter out non-specifically-bound particles, such as nanoparticles. A plurality of micropillars located within a region of a microfluidic channel forms a microfluidic hydrophobic flow layer.

In some forms, the filtration system includes a plurality of symmetrically positioned inlets leading to one or more particle bifurcation structures and/or a plurality of hydrophobic (e.g., superhydrophobic) filtration micropillar structures. In some forms, the filtration system includes a hydrophobic flow layer. In some forms, a hydrophobic flow layer provides a catchment device that facilitates the formation of a high-density array of microbeads for imaging.

### (4) Hydrophilic Interfaces

In some forms, a microfluidic channel incorporates a plurality of hydrophilic interfaces that form a “trap”, or porous mesh-like structure to selectively retain microparticles, whilst allowing elution of smaller particles, such as nano-scale particles. An exemplary trap includes a staggered regular or irregular honeycomb mesh structure. The size and configuration of the mesh structure is such that microbeads (e.g., that move within a microfluidic direction of flow through a microfluidic channel from the inlet and into the mesh structure) become lodged within the mesh. Therefore, each of the pores or spaces within the mesh are sized to retain a microscale particle. The trap structure thereby facilitates high-density particle/array formation. The mesh structure is or includes a hydrophilic coating that imparts a hydrophilic nature to the mesh or trap structure. Typically, the hydrophilic trap layer is modified with PEG reagent and exhibits hydrophilic properties in the FT junction array area. This modification permits the elution of nonspecific nanoparticles, ensuring the selective retention of target microbeads.

In some forms, the described compositions and methods differentiate hydrophobic filter inlets by modifying and perfusing hydrophilic reagents, as illustrated in the diagrams. This approach is applicable to a broad range of diverse spatial reflection symmetry microfluidic biochip designs. The described methods employ a technique focusing on altering the chemical properties of specific areas of the microfluidic biochip to ensure effective separation and analysis, tailored specifically for microfluidic biochips with a spatial reflection symmetry design in digital immunoassays.

### Hydrophilic Polymers

In some forms, the described plurality of hydrophilic interfaces within devices for performing BiZi-FIA include a plurality of surfaces coated with, or otherwise including a hydrophilic polymer. Any hydrophilic polymer can be implemented within the hydrophilic component of the described devices, including poly β-amino esters and 1,2-amino alcohol lipids. In some embodiments, the polymers are alkyl-modified polymers, such as alkyl modified poly(ethylene glycol). Other exemplary polymers include poly(alkylene glycol), polysaccharides, poly(vinyl alcohol)s, polypyrrolidones, polyoxyethylene block copolymers (e.g., PLURONIC®), polyethylene glycol (PEG) and copolymers thereof. In some forms, the hydrophilic polymers include, but are not limited to, poly(alkylene glycols) such as polyethylene glycol (PEG), poly(propylene glycol) (PPG), and copolymers of ethylene glycol and propylene glycol, poly(oxyethylated polyol), poly(olefinic alcohol), polyvinylpyrrolidone), poly(hydroxyalkylmethacrylamide), poly(hydroxyalkylmethacrylate), poly(saccharides), poly(amino acids), poly(hydroxy acids), poly(vinyl alcohol), and copolymers, terpolymers, and mixtures thereof.

In some forms, the one or more hydrophilic polymer component contains a poly(alkylene glycol) chain. The poly(alkylene glycol) chains may contain between 1 and 500 repeat units, more preferably between 40 and 500 repeat units. Suitable poly(alkylene glycols) include polyethylene glycol, polypropylene 1,2-glycol, poly(propylene oxide), polypropylene 1,3-glycol, and copolymers thereof. In some forms, the one or more hydrophilic polymer components are copolymers containing one or more blocks of polyethylene oxide (PEO) along with one or more blocks composed of other biocompatible polymers (for example, poly(lactide), poly(glycolide), poly(lactide-co-glycolide), or polycaprolactone). The one or more hydrophilic polymer segments can be copolymers containing one or more blocks of PEO along with one or more blocks containing polypropylene oxide (PPO). Specific examples include triblock copolymers of PEO-PPO-PEO, such as POLOXAMERS™ and PLURONICS™. In some forms, the hydrophilic polymer includes one or more moieties that impart distinct structural and functional properties to the polymers. For example, in some embodiments, one or more hydrophilic polymers are modified by addition of polypeptides or other small molecules. Modified hydrophilic polymers can be used to impart one or more distinct functional or structural properties to the hydrophilic component of a BiZi-FIA device, as compared to the same device in the absence of the modification. Exemplary functional or structural properties include variation of the hydrophilicity, and binding selectivity of hydrophilic component of a BiZi-FIA device.

### Polyethylene Glycol (PEG)

In some forms, the hydrophilic polymer is or includes Polyethylene glycol (PEG). PEG is one of the most commonly used hydrophilic polymer agents. The size, relative quantity and distribution of the amphiphilic PEG included in the MDNPs can influence the biophysical characteristics of the resulting modified dendrimer-based nanoparticle (MDNPs), such as structural features and charge density. In some forms, one or more physical properties of the hydrophilic interface is directly associated with the size, relative quantity and distribution of the PEG used for coating the interface (i.e., the nature and extent of pegylation that is imparted to the device). Exemplary properties that can be modified include the speed and efficacy of the retention of therapeutic, prophylactic and diagnostic agents, and the charge neutralization of the microparticles in the hydrophilic region of the device.

In some forms the PEG includes a short-chain oligo-ethylene glycol. Exemplary oligo-ethylene glycols include di-ethylene glycol, tri-ethylene glycol, tetra-ethylene glycol, penta-ethylene glycol, hexa-ethylene glycol, etc.

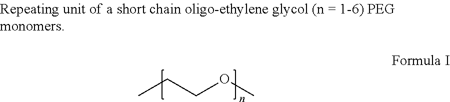

In some forms, the hydrophilic polymer is or includes monomethoxy polyethylene glycol (mPEG). In certain embodiments, the PEG or mPEG is a branched or “multi-arm” PEG. In some forms, the hydrophilic polymer is or includes polyethylene glycol polymers having different molecular weights. For example, the PEGs can have molecular weights between approximately 100 Da (i.e., PEG 100 Da) and approximately 12,000 kDa (i.e., PEG 12 KDa), inclusive. In some forms, the hydrophilic polymer is or includes a single species of PEG, or from two or more different species of PEGs. In some forms, the hydrophilic polymer is or includes a single polymer species, or a mixture of multiple different polymer species. In some forms, the hydrophilic polymer is modified, for example, with one or more adducts. For example, hydrophilic polymers can be modified with the same or different adducts. In some forms, the hydrophilic polymer is or includes the amphiphilic polymer DSPE-mPEG. In some forms, the hydrophilic polymer is or includes DSPE-mPEG molecules having different molecular weight mPEG, such as DSPE-mPEG (350); DSPE-mPEG (550); DSPE-mPEG (750); DSPE-mPEG (1000); DSPE-mPEG (2000); DSPE-mPEG (3000); or DSPE-mPEG (5000). The lipidic component can include saturated or non-saturated fatty acidic moieties.

### (5) High Density Bead Array (HDBA)

The BiZi-FIA devices include one or more structures that concentrate a plurality of microbeads within one or more defined areas of the microfluidic channel, to form a microbead array. In some forms, the array is a High Density Bead Array (HDBA). The described BiZi-FIA systems use a HDBA to capture and isolate beads-based immunocomplex and identify them with ultra-bright fluorescent labels. Typically, each bead filter (BD) is an FT junction node between a lyophobic and hydrophilic conduit with a height difference. The FT junction precisely controls single-bead immobilization at a certain size and filtration with a certain target molecule. The BiZi-FIA with HDBDA thereby enables sample handling without pumps, reduced costs, and enhanced sensitivity compared to traditional digital Elisa.

The array is typically formed by immobilization of the solid phase matric (e.g., microbeads) within the mesh-like “trap” structure of the BiZi-FIA. In an exemplary form, the array of microbeads includes from about 1×10⁴ to about 1×10⁶ beads, inclusive on a 2D plane (e.g., an area of about 5×5 mm²), for example, about 4×10⁵ of beads on a 2D plane (e.g., an area of about 5×5 mm²) to enable imaging and analysis. In some forms, the location and dimensions of an array area correspond with a field of view diameter of about 6.25 mm (e.g., using a Nikon ECLIPSE Ti2, 20 mm FOV).

The number of multiplexed assays possible is limited by the size of the array, the camera field of view, and the ability of the optical system to discern unique fluorescent signals on the array reliably. In some forms, the upper limit of the number of wells that can be imaged using a CCD camera is approximately 200,000. Therefore, about 200,000 wells are typically available for each assay to maintain dynamic ranges of multiplexed assays.

### (6) Multi-Layered Configuration

In some forms, the chip includes a multiple-layered structure. For example, in some forms, a chip includes a two-layered structure. An exemplary two-layer structure includes a first layer and a second layer. In an exemplary form, a first layer is an upper layer and a second layer is a lower layer. For example, in some forms, an upper layer is positioned directly on top of, or directly above a lower layer. For example, in some forms, an upper layer contacts a lower layer. Therefore, in some forms, the lower surface of an upper layer contacts the upper surface of a lower layer to form a two-layer structure. In some forms, both of the upper and lower structures of a two-layered chip are the same or different size. For example, in some forms, both of the upper and lower structures of a chip include the same or different surface area. In some forms, both of the upper and lower structures of a chip combine to form the top and bottom sections of the one or more microfluidic channels.

### Exemplary First Layer Structures

In some forms, the first layer includes a plurality of symmetrically positioned inlets leading to one or more particle bifurcation structures and/or a plurality of hydrophobic (e.g., superhydrophobic) filtration micropillar structures. In some forms, a first layer is a hydrophobic flow layer. In some forms, a first layer is an upper layer. Therefore, in some forms, an upper layer is a hydrophobic flow layer.

### Exemplary Lower Layer Structures

In some forms, the second layer incorporates the hydrophilic interface component of a RTJ structure within a BiZi-FIA device. For example, in some forms, a lower layer combines with an upper layer to form a “trap”, or porous mesh-like structure that selectively retains microparticles, whilst allowing elution of smaller particles, such as nano-scale particles. An exemplary trap includes a staggered regular or irregular honeycomb mesh structure with a staggered honeycomb mesh structure. When the second layer includes a staggered honeycomb mesh structure, the second layer facilitates high-density particle array formation. Therefore, in some forms, a second layer is a hydrophilic trap layer.

### (ii) Exemplary BiZi-FIA Chip Design

An exemplary platform for BiZi-FIA is a biochip for single-molecule protein detection includes a two-layer structure, having a first (upper) hydrophobic flow layer and a second (lower) hydrophilic trap layer, whereby the lower surface of the upper layer contacts the upper surface of the lower layer to form a single, contiguous microfluidic channel, the base of which includes the trap. In the first (upper) hydrophobic flow layer, particles (such as microparticles) navigate through a filtering fractal structure including a plurality of featuring nonspecific adsorption properties with hydrophobic surface, capture and filter out the nanoparticles. The second (lower) hydrophilic trap layer is modified with PEG reagent and exhibits hydrophilic properties in the FT junction array area. This modification is instrumental in the elution of nonspecific nanoparticles, ensuring the selective retention of target microbeads. Single protein molecules are captured on the microbeads and labelled with Qt dots nanoparticles. Simultaneously, gold nanoparticles bound within a streptavidin-affinity system are introduced to act as “amplifying antennas” for the fluorescent signal. This dual labelling approach-using quantum dots for initial marking and gold nanoparticles for signal enhancement.

Concurrently a control area features a structure that is a mirror reflection of the test area. By manipulating a single variable, the control area conducts tests in control samples. An exemplary FT junction array is sized with an upper limit of 6×6 mm², with each of microbeads array, imaging area is 30 mm². Images are obtained in two conduits to identify the “on” beads, and the “off” beads. Fluorescence signals detected the blank sample of the FT junction array are considered background noise, guiding an adaptive algorithm to minimize system errors. Under varying environmental conditions, there is no need to repeatedly test the standard curve. This indicates a robust assay or measurement system that maintains its accuracy and consistency despite changes in the environment, thereby reducing the need for frequent recalibration or reassessment of the standard curve. This feature is particularly valuable in ensuring time-efficiency and reliability in ongoing or long-term experimental or analytical setups.

An exemplary platform for BiZi-FIA is depicted in FIGS. 1A-1D.

(iii) Solid Phase Matrix

In some forms, the BiZi-FIA is configured for the attachment of target biomarkers to capture probes that are complexed with or conjugated to a solid phase matrix. Typically, the BiZi-FIA is configured for the retention of a micro-scale matrix. An exemplary micro-scale solid phase matrix is a particle, such as a microparticle. Micro-scale particles, or “microparticles” are sized between about 1.0 and 1000.0 about micrometers, inclusive, such as about 1 μm, or 2, 3, 4, 5, 6, 7, 8, 9, 10, 20, 30, 40, 50, 60, 70, 80, 90, 100, 150, 200, 300, 400, 500, 600, 700, 800, 900, or 999 or 1,000 μm. The hydrodynamic diameter (Dh) of a molecule is defined as the diameter of a perfect solid sphere that would exhibit the same hydrodynamic friction as the molecule of interest. For example, the Dh value reflects primarily the hydrodynamic friction but is usually also a good estimation of the absolute size of the molecule. For example, in some forms, a microparticle has a hydrodynamic volume (Dh) of between about 1.0 and 1000.0 about micrometers, inclusive, such as about 1 μm, or 2, 3, 4, 5, 6, 7, 8, 9, 10, 20, 30, 40, 50, 60, 70, 80, 90, 100, 150, 200, 300, 400, 500, 600, 700, 800, 900, or 999 or 1,000 μm. in other forms, a microparticle has a diameter of between about 1.0 and 1000.0 about micrometers, inclusive, such as about 1 μm, or 2, 3, 4, 5, 6, 7, 8, 9, 10, 20, 30, 40, 50, 60, 70, 80, 90, 100, 150, 200, 300, 400, 500, 600, 700, 800, 900, or 999 or 1,000 μm. In an exemplary form, microbeads have a diameter of about 3 μm.

In some forms, the microparticles are formed of or include a polymer, such as an inert polymer. In some forms, the microparticles are formed of or include a metal. In some forms, the microparticles are formed of or include glass. In some forms, the microparticles are opaque. In some forms, the microparticles are transparent or permit passage of one or more different wavelengths of light throughout the particle. In some forms, the microbeads are biotinylated beads. In some forms, the number of beads used should be in excess of the number of molecules of a target biomarker within the sample. For example, in some forms, the number of microbeads is at least the same as, or greater than the number of molecules of a target biomarker within the sample. In some forms, the number of microbeads is 100%, 150%, 200%, 300%, 400%, 500%, 600%, or more than 600% of the total number of molecules a target biomarker within the sample.

An exemplary solid phase matrix includes Dynabeads, such as Dynabeads including a 2.8 μm diameter carboxylic acid and epoxy-linked superparamagnetic beads. In an exemplary form, the solid phase matric is or includes between about 100,000 and 1,000,000 microbeads. An exemplary number of microbeads is about 450,000 microbeads. Typically, the beads are conjugated to one or more capture agents that are specific for one or more target biomarkers.

Without the constraints of magnetic forces, microbeads that exhibit excellent uniformity and optical transparency are a preferred choice, including silica, polystyrene, glass microbeads, gold nanoparticle beads. The controllable and uniform capture and distribution of these microbeads further minimize random errors. The consistent behaviour and transparency of the microbeads contribute to more precise measurements and clearer observation in assays or analytical procedures.

### (a) Capture Agents

Typically, the described BiZi-FIA devices incorporate capture agents for specific binding to a target biomarker. The capture agents are typically nano-scale particles, however capture agents can include, or can be conjugate to a solid-phase matrix, such as a bead. Exemplary capture agents include, but are not limited to immunoglobulins, nucleic acids such as DNA and RNA, such as RNA aptamers, and small molecule, such as ligands for specific proteins and/or agents. The term “capture probe” refers to any molecule capable of capturing (directly or indirectly) and/or labelling a target molecule (e.g., an biomarker of interest) in a biological sample. In some forms, the capture probe is a nucleic acid or a polypeptide that includes at least one analyte capture sequence. In some forms, the capture probe includes a capture tag, for example for conjugation with the solid phase matrix.

### (1) Capture Tags

In some forms the capture agent includes one or more capture tags, for example, to couple the capture agent to a solid support matrix, or another molecule. Preferably, the capture tag is a compound, such as a ligand or hapten, which binds to or interacts with another compound, such as ligand-binding capture agent or an antibody capture agent.

It is also preferred that such interaction between the capture tag and the capturing component be a specific interaction, such as between a hapten and an antibody or a ligand and a ligand-binding molecule.

A preferred capture tag is biotin. In an exemplary form, the capture agent is a biotinylated capture agent. In a preferred form the biotinylated capture agent is a biotinylated antibody.

Capture tags incorporated into capture agents allow the capture agents to be captured by, adhered to, or coupled to a substrate, such as a transparent microbead.

### (b) Reporter Molecules

Typically, the described BiZi-FIA devices incorporate reporter molecules, such as nanoscale fluorescent markers or labels, for detection of biomarkers. Reporter molecules and labels are known in the art. Any nano-scale reporter molecules known in the art can be included in the described systems for BiZi-FIA measurement of biomarkers.

Exemplary reporter molecules for use in the described BiZi-FIA systems include luminescent quantum dot nanoparticles. An exemplary quantum dot nanoparticle has a diameter of about 120 nm. An exemplary quantum dot nanoparticle has a 625 nm Emission Peak. Typically, gold nanoparticles bound within a streptavidin-affinity system are introduced to amplify the fluorescent signal. This dual labeling approach-using fluorescent labels for initial marking and gold nanoparticles for signal enhancement, increases the overall accuracy and sensitivity of the system.

Typically, the reporter molecule has an affinity for the capture agent that has bound to a target biomarker. Typically the reporter molecule is sized according to the pores within the hydrophilic trap/mesh section of the RTJ structure of the described BiZi-FIA devices, such that the unconjugated reporter labels are washed away from the conjugated microparticles within the BiZi-FIA device. In some forms, the bandwidth of the optical system limits the number of available dyes to about four (e.g, red, green, blue, yellow). In some forms, the reporter includes one or more molecules that act as a detectable label or dye. In some forms, the label is an optically-detectable moiety (e.g., a fluorophore). Non-limiting examples of types of optically-detectable labels include a fluorescent, chemiluminescence, or electrochemically luminescent label. Examples of fluorescent labels include, but are not limited to, 4-acetamido-4′-isothiocyanatostilbene-2,2′disulfonic acid; acridine and derivatives thereof such as acridine, acridine isothiocyanate; 5-(2′-aminoethyl)aminonaphthalene-1-sulfonic acid (EDANS); 4-amino-N-[3-vinylsulfonyl)phenyl]naphthalimide-3,5disulfonate; N-(4-anilino-1-naphthyl) maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 15 1); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylaminolnaphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivatives of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalocyanine; naphthalocyanine; any of the fluorescent labels available from Atto-Tec, such as Atto 390, Atto 425, Atto 465, Atto 488, Atto 495, Atto 520, Atto 532, Atto 550, Atto 565, Atto 590, Atto 594, Atto 610, Atto 611X, Atto 620, Atto 633, Atto 635, Atto 637, Atto 647, Atto 647N, Atto 655, Atto 680, Atto 700, Atto 725, Atto 740, etc.; any of the fluorescent labels available from Dyomics such as DY-630, DY-631, DY-632, DY-633, DY-634, DY-635, DY-636, Dy-647, Dy-648, DY-649, Dy-650, Dy-651, DY-652, etc.; any of the fluorescent labels available from Pierce such as DyLight 405, DyLight 488, DyLight 549, DyLight 633, DyLight 649, DyLight 680, DyLight 800, etc.; any of the fluorescent labels available from AnaSpec such as HiLyte Fluor™ 488 dyes, HiLyte Fluor™ 555 dyes, HiLyte Fluor™ 647 dyes, HiLyte Fluor™ 680 dyes, HiLyte Fluor™ 750 dyes, HiLytePlus™ 555 dyes, HiLytePlus™ 647 dyes, HiLytePlus™ 750 dyes, etc.; any of the fluorescent labels available from Denovo Biolables such as Oyster 500, Oyster 550 P, Oyster 550 D, Oyster 556, Oyster 645, Oyster 650 P, Oyster 650 D, Oyster 656, etc.; IRDye® 680, IRDye® 700, IRDye® 700DX, IRDye® 800, IRDye® 800 RS, IRDye® 800 CW, etc.; any of the fluorescent labels available from SETA Biomedicals such as Seta K1-204, Seta K5-3212, Seta K8-1342, Seta K8-1352, Seta K8-1357, Seta K8-1407, Seta K8-1642, Seta K8-1644, Seta K8-1663, Seta K8-1664, Seta K8-1669, Seta K8-3002, Seta K4-1082, Seta K8-1669, Seta K7-545, Seta K7-547, Seta K7-549, Seta K8-1252, Seta K8-1261, Seta K8-1262, Seta K8-1320, Seta K8-1344, Seta K8-1367, Seta K8-1377, Seta K8-1382, Seta K8-1446, Seta K8-1667, Seta K8-1752, Seta K8-1762, Seta K8-1767, Seta K8-1777, Seta K8-1782, etc.

### (iv) Samples

The described BiZi-FIA devices are designed for the detection and measurement of or one or more biomarkers, for example, a biomarker present within a sample. An exemplary sample is a biological sample, such as a biological fluid. An exemplary biological fluid is a bodily fluid, for example, a bodily fluid obtained from a sample.

The described BiZi-FIA devices require a microliter amount of a sample. Therefore, in some forms, a sample for use according to the described devices for BiZi-FIA have a volume of 1 μL, or 2, 3, 4, 5, 6, 7, 8, 9, 10, 20, 30, 40, 50, 60, 70, 80, 90, 100, 150, 200, 300, 400, 500, 600, 700, 800, 900, or 999 or 1,000 μL, or more than 1,000 μL. In an exemplary form, a sample is or is diluted to a volume of between about 40 μL and about 60 μL solution, inclusive. In some forms, the sample volume is 50 μL.

In some forms, a sample is an unfiltered bodily fluid, such as pus, blood or tears from a subject. In some forms, a sample is a concentrated sample, for example, concentrated to less than 100% of the initial (“neat”) volume, such as 90%, 80%, 70%, 60%, 50%, 40%, 30%, 20% of 10%, or less than 10% of the initial volume of a sample such as a bodily fluid. In other forms, a sample is diluted, i.e., to increase the volume relative to the initial (“neat”) volume of a sample such as a bodily fluid. For example, in some forms a sample is diluted to more than 100% of the initial (“neat”) volume, such as 900%, 800%, 700%, 600%, 500%, 400%, 300%, 200% or 150%, or less than 150% but more than 100% of the initial volume of a sample such as a bodily fluid.

Typically, the volume of a neat biological sample, such as a bodily fluid, that is required for BiZi-FIA detection and/or measurement of a target biomarker by the described devices is between about 0.01 μL and about 5.0 μL, inclusive, such as about 0.1375 μL per biomarker. As demonstrated in the Examples, in an exemplary form, the volume of 2.2 μL of human tear fluid is diluted to 16 parts to meet the requirement of 8 different biomarker detection (i.e., a volume of about 0.1375 μL per biomarker).

In some forms, the amount of a target biomarker within a neat biological sample, such as a bodily fluid, is between about 1 IU/mL and about 10 U/mL, inclusive, such as about 1 U/mL. It boasts a detection sensitivity extending to attomole levels for model assays and low femtomolar levels in human tears. The described BiZi-FIA devices can detect an amount of a target biomarker in a sample at a concentration from about 10×10⁻¹⁸ M to about 10×10⁻⁵ M, inclusive.

### (a) Biological Fluids

In some forms, one or more biomarkers are within a biological sample, such as sample that is or includes a biological fluid. Typically, the biological sample includes a bodily fluid from a subject. Generally, the subject is a mammal. In some forms, the subject is a human. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid. In some forms, the biological sample includes tear fluid, urine, or serum obtained from a subject. In other forms, the biological sample includes blood, sputum, mucus, or a liquidized or lysed tissue, such as a lysate of bone marrow, or peripheral blood cells.

### (b) Target Biomarkers

The described BiZi-FIA devices are designed for the detection and measurement of or one or more biomarkers, for example, a biomarker present within a biological sample. Any biomarker known in the art can be detected according to the described BiZi-FIA methods. For example, a target biomarker can be or include a small molecule, peptide, carbohydrate, lipid, nucleic acid (such as DNA or RNA), a protein (such as an enzyme, immunoglobulin or receptor), a synthetic polymer, a metal, a hormone, drug, bacteria, virus, protozoan or combinations thereof. In some forms, the biomarker is a cytokine, or an antigen, such as a cancer antigen or an antigen derived from a microorganism, such as a pathogen, or an allergen. In some forms, the target biomarker is an immunoglobulin, such as immunoglobulin specific for an antigen, such as an autologous antigen or an exogenous antigen. Exemplary biomarkers include TNF-α, LCN-1, NFL, tau-n, IgE, for example, as inBiZi-FIAtive of an ocular inBiZi-FIAtion present within human tear fluid. An exemplary pathogenic biomarker is a viral protein, such as a SARS-COV2 N protein as inBiZi-FIAtive of an infection with a SARS-COV2 virus. Other exemplary biomarkers include tumor biomarkers, such as Prostate-Specific Antigen (PSA), Carcinoembryonic Antigen (CEA), Alpha-Fetoprotein (AFP), Cancer Antigen 125 (CA-125), Cancer Antigen 19-9 (CA 19-9), Human Epidermal Growth Factor Receptor 2 (HER2/neu), Cytokeratin 19 Fragment (CYFRA 21-1), B-Raf Proto-Oncogene (BRAF V600 Mutation), and Epidermal Growth Factor Receptor (EGFR Mutation).

### (v) Buffers and Wash Reagents

In some forms, methods for microfluidic device-based BiZi-FIA employ buffers and wash reagents. Buffers and Wash reagents can be any solution that is used to remove or reduce the local concentration of another component, for example, a contaminant.

Exemplary buffers and wash reagents include water, physiological salt solutions, for example, PBS, and DMEM.

### (a) Buffers

In some forms, methods for microfluidic device-based BiZi-FIA employ buffers for diluting samples, loading of microparticles conjugated with capture agents with target biomarkers, and for actuating flow within a BiZi-FIA device. Typically, the methods include wash reagents and/or buffers that are known in the art to be physiological and/or do not disrupt the structure or amount of a target biomarker.

In some forms, the buffer includes one or more of EDTA, sodium, ammonium, chloride, iodide, phosphate ions, and TRIS buffer.

### III. Methods of Making

Methods for fabricating digital immune-chromatography (BiZi-FIA) chips have been developed.

Typically, the methods include the steps of
(a) Lithography to ablate a simple microconduit pattern on a silicon wafer;
(b) Surface treatment of the microconduit;
(c) Capture agent coupling with reporter molecule; and
(d) Coupling capture agent on beads.

### A. Lithography

The methods include one or more steps of lithography. In an exemplary form, PDMS and a hardener are mixed in a 10:1 weight ratio and the mixture is de-gassed in a vacuum to remove air bubbles.

In some forms, the PDMS mixture is poured onto the ablated silicon wafer and cured, e.g., at 70° C. for 30 minutes. After curing, the PDMS layer is peeled off from the silicon wafer to form a PDMS convex mold.

In some forms, the methods treat the PDMS mold with 02 plasma for 1 minute and then immerse it in a 0.1 M PEG solution (average molecular weight 6000 g/mol) to form an isolation film.

In some forms, the methods repeat the PDMS pouring and curing steps on the convex mold to create a concave PDMS mold.

In some forms, the methods bond the PDMS mold to a blank PDMS substrate to form the microfluidic chip.

### B. Surface Treatment

The methods include one or more steps of surface treatment. In some forms, the methods prepare a Surface Treatment Reagent. The surface treatment methods enhance the hydrophilicity of the PDMS surfaces in the microfluidic chip, thereby minimizing nonspecific adsorption and improving chip performance for various applications.

For example, in some forms, the methods prepare a 0.1 M solution of Polyethylene Glycol (PEG) with an average molecular weight of 6000 g/mol. This solution serves as the surface treatment reagent.

In some forms, the methods include Setting Up the Surface Treatment System. For example, in some forms, the methods arrange a surface treatment pool connected to the inlet of the surface treatment conduits in the microfluidic chip.

In some forms, the methods include one or more steps to ensure the system is sealed and leak-proof. For example, in some forms, the methods include connecting a precision pump to the surface treatment pool to control the flow of the PEG solution.

In some forms, the methods initiate the Surface Treatment Process. For example, in some forms, the methods activate the precision pump to introduce the hydrophilic solution, such as a PEG solution, into the surface treatment conduits. The flow rate should be maintained at a consistent and controlled pace to ensure even distribution of the reagent. In some forms, the PEG solution simultaneously flows through the array capture conduits, which are parallel to the surface treatment conduits. This ensures uniform exposure of the conduits to the treatment reagent.

In some forms, the methods maintain Flow and Treatment Time. For example, in some forms, the methods maintain a constant flow rate of the PEG solution throughout the treatment process. Recommended flow rate parameters can be around 0.5 mL/min, but this may vary based on the specific design and dimensions of the microfluidic chip.

The duration of the surface treatment process should be carefully timed. A typical treatment time can range from 25 to 30 minutes. This duration ensures adequate surface modification without overexposure.

In some forms, the methods Complete and Discharge the Reagent. For example, in some forms, after the treatment duration is completed, the methods gradually decrease the flow rate and eventually stop the pump.

The methods allow the hydrophilic (i.e., PEG) solution to be completely discharged from the exit port of the microfluidic chip. Ensure that no residual treatment reagent remains within the conduits.

In some forms, the methods include post-Treatment Handling. For example, in some forms the methods include steps to rinse the surface-treated conduits with deionized water to remove any unbound PEG molecules. In some forms Dry the microfluidic chip under a gentle stream of nitrogen to remove any remaining moisture.

In some forms, the methods include verification of Surface Treatment. In some forms, the methods assess the success of the surface treatment by measuring the contact angle of a water droplet on the treated surface. A significant decrease in contact angle indicates successful hydrophilic modification.

Optionally, in some forms, the methods include additional analytical techniques such as SEM (Scanning Electron Microscopy) or FTIR (Fourier Transform Infrared Spectroscopy) for further verification and analysis of the surface characteristics. In some forms, the methods include using a CCD camera to measure the contact angle of a 5 μL droplet of deionized water on the differently treated PDMS substrates. In some forms, the methods evaluate the hydrophilic property of the treated surface by recording the contact angle over up to 420 hours for the plasma-PEG treated surface. In some forms, the methods examine the morphology of the PDMS before and after surface treatment using a SEM microscope.

In some forms, the methods employ FTIR (Fourier Transform Infrared Spectroscopy) to compare the PDMS samples before and after treatment.

C. Capture Agent Coupling with Reporter (Label)

The methods include one or more steps of coupling a capture agent with a reporter molecule.

In some forms, the methods include preparing a capture agent, such as a detection antibody, by coupling with a reporter, for example with a streptavidin-functionalized quantum dot nanoparticle.

In some forms, the methods include one or more steps to conjugate a capture agent specific for a target biomarker with a reporter molecule according to any of the standardized protocols known in the art.

In some forms, the methods include one or more steps to couple antibody to quantum dot nanoparticles. In some forms, the methods prepare antibody-conjugated quantum dot nanoparticles primed and ready for integration into further experimental applications, such as immunofluorescence assays or protein detection.

As demonstrated in the Examples, in some forms, the methods include one or more steps to prepare a working solution of streptavidin quantum dot nanoparticles with a 625 nm emission peak was prepared according to the manufacturer's instructions (catalog number of DNQ-N008, DiagNano™, CD Bioparticles). The prepared bead solution (50 μL) was centrifuged at 5000 rpm for 2 minutes at room temperature to remove the supernatant. Following this, the beads underwent a thorough washing process in which they were gently vortexed and then centrifuged three times using 1 mL of phosphate-buffered saline (PBS). Subsequently, 25 μg of the antibody solution was introduced to the washed beads. The tube containing the mixture was gently pipetted to ensure uniform mixing. The tube was then incubated at room temperature with gentle shaking on an orbital shaker for 1 hour, allowing sufficient time for the antibodies to bind effectively to the streptavidin-coated Qt nanoparticles. Once the incubation period was completed, the supernatant was removed after centrifugation. After that, the beads were subjected to three additional washes using 1 mL of PBS each time to remove any unbound antibodies from the beads. After the final wash, the antibody-coupled Qt nanoparticles were resuspended in 100 μL of PBS for immediate use or in a suitable storage buffer for longer-term storage.

### D. Coupling Capture Agent on Beads

In some forms, the methods include one or more steps to couple a capture agent, such as a capture agent conjugated to a label, with a solid phase matrix. In some forms, the methods include one or more steps of coupling a labeled capture agent with a microbead.

In some forms, the methods include one or more steps to conjugate a labeled capture agent specific for a target biomarker with a microparticle according to any of the standardized protocols known in the art.

As depicted in the examples, a method was elucidated for covalently immobilizing antibodies onto the surface of DYNABEADS® M-270 Epoxy. The protocol described in the Examples was designed and executed strictly in accordance with the guidelines provided in the Antibody Coupling Kit from Thermo Fisher Scientific, DYNABEADS® (2.8 μm diameter carboxylic acid and epoxy-linked superparamagnetic beads).

Initially, a series of buffers and solutions, including C1 for bead washing and preparation, C2 as an activator for coupling, LB as a low-salt buffer for non-specific binding removal, HB as a high-salt buffer to enhance binding, and SB for long-term bead storage, were prepared by Antibody Coupling Kit. These reagents, of analytical grade, were confirmed to be compatible with protease and phosphatase inhibitors and were stored at 2-25° C.

For a 5 mg bead preparation, exactly 5 mg of DYNABEADS® M-270 Epoxy was weighed out with beads at room temperature to prevent condensation. The beads were then washed using the C1 solution to ensure their surface was ready for coupling. After magnetic separation, a volume of antibody was added, and the remaining volume to reach 250 μl was filled with C1. An additional 250 μl of the C2 activator solution was introduced, facilitating the covalent attachment of the antibodies to the bead surface. The mixture was then incubated on a roller at 37° C. overnight.

The next day, the tube was placed on a magnet, and the supernatant was discarded. The beads underwent sequential washes with HB and LB to remove non-specifically bound substances and enhance specific binding. A short SB wash was conducted, followed by a long SB wash where the beads were incubated at room temperature for 15 minutes. The beads were finally resuspended in 500 μl of SB, ensuring their stability and longevity. In an exemplary forms, the final bead concentration is 10 mg antibody coupled beads/ml.

### IV. Methods of Digital Immunochromatography (BiZi-FIA)

Methods for digital immune-chromatography (BiZi-FIA) have been developed. Typically, the methods utilize a BiZi-FIA chip device, loaded with functionalized beads as solid-phase carriers for specific capture molecules. These beads are guided through a microfluidic conduit, where the filter-like flow control ensures the precise manipulation and directed movement of the beads. As the beads encounter the trap junction, they are immobilized, allowing for controlled interactions between the target analytes and the capture molecules.

The filter-like flow and trap junction mechanism offer several advantages over conventional immunochromatography techniques. The controlled flow enables accurate control over reaction times, improving the binding kinetics between the capture molecules and target analytes. The trap junction provides a stable and confined environment for the immobilization of the beads, facilitating enhanced sensitivity and specificity in analyte detection.

The integration of filter-like flow and trap junction technology, and bead-based assays offers a versatile and robust platform for digital immunochromatography. The method holds great potential in various fields, such as clinical diagnostics, environmental monitoring, and food safety, due to its high sensitivity, rapid detection, and potential for cost-effective, high-throughput analysis. This digital immunochromatography approach provides a sensitive and multiplexed analyte detection.

The described BiZi-FIA biochip simplifies the immunoassay process by integrating incubation, filtration, and washing steps within the chip. The facile BiZi-FIA achieves single molecule protein detection with attomole sensitivity, marked by enhanced signal-to-noise ratio, rapid assay times, reduced reagent usage, and reduced setup cost. The robustness of the assay methods technique is demonstrated in the Examples, which exemplify a 94% capture efficiency for 4×10⁵ beads within a compact area (30 mm²), achieved in 180 seconds, which capture efficiency and array density is significantly two times higher than commercialized single-molecule array chips (e.g., SimoA chips).

If the background noise in the control area exceeds the error range of the standard curve, the test is considered a failure. If the background noise is within the acceptable range, the signal from the testing area can be adjusted by subtracting the background noise from the control area, yielding the effective signal for concentration estimation based on the standard curve.

Typically, the methods include one or more steps using BiZi-FIA to detect and/or measure the amount of a biomarker within a sample. The assay methods typically include one or more steps of
(a) providing, on the described microfluidic device for BiZi-FIA
   (i) a fluid sample including a target biomarker, and
   (ii) a control sample including a known amount of the target biomarker;
   wherein the fluid sample and the control sample are provided in separate microfluidic channels in the microfluidic device;
   (iii) a solid phase matrix including a plurality of microparticles,
   wherein a microparticle of the plurality of microparticles includes a plurality of capture agents conjugated thereto,
   wherein a capture agent of the plurality of capture agents is specific for the target biomarker; and
   (iv) a labeled capture agent specific for the target biomarker,
   wherein an equal amount of the microparticles are provided in separate microfluidic channels in the microfluidic device;
(b) actuating movement of the microparticles within the microfluidic channels in the microfluidic device,
   wherein the movement includes filtration and washing of the microparticles within the flow-trap junctions within the microfluidic channels in the microfluidic device; and
(c) forming an array including the microparticles within the trap of each of the microfluidic channels in the microfluidic device; and
(d) imaging the array of microparticles within the microfluidic device.

In some forms, the methods further include one or more steps for
(e) detecting and measuring the target biomarkers conjugated or complexed with microbeads within the array.

### A. Providing Reagents

Typically, the assay methods include the step of providing, on the described microfluidic device for BiZi-FIA an effective amount of a fluid sample including a target biomarker.

In some forms, the fluid sample includes a volume of between 10 μL and 100 μL. In some forms, the control sample includes a volume of between 10 μL and 100 μL.

In some forms, the fluid sample and the control samples are contacted with microbeads conjugated with a capture agent specific for the target biomolecule prior to providing the test and control samples or the microbeads on the device. For example, in some forms, the microbeads conjugated to the labeled capture agents specific for the target biomarker(s) are incubated with the test or control sample prior to providing the reagents of the BiZi-FIA device to enable specific binding of the target biomarker to the capture agent on the surface of the microbead.

In some forms, the fluid sample and the control samples are contacted with microbeads conjugated with a labeled capture agent specific for the target biomolecule for an amount of time effective to enable binding of at least 50%, at least 60%, at least 70%, at least 80%, at least 90%, or at least 100% of the biomarker molecules within the sample to the capture agents immobilized on the microparticles. In some forms, the incubation time is between 1 second and 1 minute, or between 1 minute and about 100 minutes or 100 hours. In an exemplary form, the incubation time is about 30 minutes. For example, in some forms, after ensuring thorough mixing, the samples are incubated for 30 minutes at room temperature. The incubation period generally facilitates the binding of quantum dot-labeled capture agents, such as detection antibodies, to the antigens previously captured on the magnetic beads.

In an exemplary form, after incubation, a further washing step is performed following the same protocol, i.e., to remove unbound quantum dot-antibody complexes. In an exemplary form the beads are separated from the supernatant using a magnet and resuspended in 50 μL buffer.

In an exemplary form, the resuspended bead-protein-quantum dot complexes are then carefully introduced into the microfluidic BiZi-FIA device, for example, using a micropipette.

Typically, an equal amount of the microparticles are provided in separate microfluidic channels in the microfluidic device.

B. Actuating Movement of the Microparticles within the Microfluidic Channels in the Microfluidic Device

Typically, the assay methods include the step of actuating the flow of the particles within the microfluidic channels in the device. The flow can be automated, for example, by use of pumps, or it can be manually actuated, for example, by a pipette. The flow is typically actuated by applying a pressurized fluid into the inlets within the device. Excess fluid passes through the device via the outlet conduits within the trap structure.

The amount of fluid and pressure and time can be varied according to the requirements of the assay. In some forms, the methods pass a fluid through the device in an amount effective to filter and wash the microparticles within the device. In some forms, the methods pass a fluid through the device in an amount and for a time effective to concentrate the microparticles within the trap structures of the device to form an array. In some forms, the methods pass a fluid through the device to filter, concentrate and/or wash the microparticles in the fluid for a time of between about 60 and about 300 seconds, inclusive. In some forms, the methods pass a fluid through the device to filter, concentrate and/or wash the microparticles in the fluid for a time of between about 180 seconds.

### C. On-Bead Filtering and Washing

In some forms, methods for using the described BiZi-FIA platforms include automated filtering and washing of capture beads.

In some forms, the methods include one or more steps to wash the beads, to efficiently remove non-specific impurities while preserving specific immunocomplexes on the beads. For example, in some forms, the methods load and isolate microbeads associated with single enzyme molecules using only hydrodynamic forces. Parallel perfusion of multiple channels significantly enhances efficiency. In some forms, upon entering the intermediate array region, single molecule immunocomplexes captured by the beads interact with the contrasting surface properties of the array region. The hydrophilic and anti-static surfaces prevent non-specific adsorption of quantum dot (QD) microspheres, ensuring their removal from the chip through fluid dynamics. The methods flow particles past a hydrophobic polymer, such as Polydimethylsiloxane (PDMS), in the flow separation zone, to effectively eliminate non-specifically bound proteins and impurities, and thereby significantly improving the signal-to-noise ratio (SNR) and detection sensitivity. Therefore, the methods employ on-chip separation, whereby interaction of particles with hydrophobic micropillars bind and remove non-specifically adsorbed particles. The methods employ collision-based filtering by passing the microbeads over hydrophilic and hydrophobic surfaces to reduce non-specific adsorption of particles to the beads.

### D. Bead Detection

The described methods include one or more steps for detecting a biomarker based on imaging microbeads within a high density microbead array have been developed. In some forms, the methods employ fluorescence detection, where beads are arrayed in a detection chamber. In some forms, the methods employ fluorescence detection if a negative control sample (0 fg/mL). In some forms, the methods automate filtration processes to improve bead arraying using the BiZi-FIA platform. In some forms, where the BiZi-FIA platform employs silica beads as the primary capture substrate, the higher optical clarity of silica beads and reduced tendency for non-specific binding, lower background noise compared with non-silica bead systems. In some forms, measurement of optical intensity is carried out by fluorescence spectrophotometer at 611 nm. In some forms, measurement includes microscopic imaging.

In an exemplary form, the methods employ one or more algorithms for accurate measurement of biomarker quantity. An exemplary algorithm is an adaptive difference algorithm with one-step imaging for accurate bead detection.

### 1. Adaptive Difference Algorithm

In some forms, the methods for detection and quantitation of biomarkers include an adaptive difference algorithm.

Capitalizing on the excellent sample loading capabilities of BiZi-FIA, an adaptive differential algorithm has been designed to compensate for background noise fluctuations. The algorithm operates by contrasting the signals from the control sample with those of the fluid sample, effectively cancelling out background noise signals that are present under identical environmental conditions. This methodology maintains assay accuracy, particularly when detecting ultra-low analyte concentrations, and it demonstrates significant resilience to environmental variations. BiZi-FIA obviates the necessity for conductive metal film deposition on microchip surfaces, a requirement in traditional methodologies that employ di-electrophoresis (DEP), electric, or magnetic field techniques to augment bead loading rates. The simplification achieved with BiZi-FIA enables high-resolution, bead-based analyte quantification with ease of operation.

In some forms, the number of beads used and their percentage to be analyzed are two important parameters for the sensitivity and dynamic range of the described BiZi-FIA assay. In some forms, the number of beads used determines the fₒₙ (the number of positive events over the total number of beads) and the AEB molecules. For example, for 1,000,000, 500,000, and 100,000 beads used, the theoretical AEB is 0.0006, 0.0012, and 0.0060, respectively. As a result, using fewer beads will lead to a higher fₒₙ and AEB. In some forms, the percentage of beads analyzed leads to the sensitivity of the detection, as the more beads are analyzed, the lower the measurement uncertainty.

In some forms, when all beads in an array are analyzed, the number of beads used does not affect the sensitivity of detection. In other forms, the more beads used, the shorter the incubation time required to fully assay. In some forms, using a larger number of beads expands the dynamic range of quantitative detection, enabling the chip to be applicable in a larger range of analyte concentrations. Therefore, in some forms, using as many beads as possible is advantageous for improving sensitivity if all the beads are analyzed. While analyzing so many beads may be advantageous, it may also result in more complex systems and instrumentation that are not amenable to routine or rapid use. Therefore, by increasing the density of the beads array, reaction and imaging time can be reduced, enhance detection sensitivity with simplified system complexity, and enable rapid point-of-care diagnosis.

It may be that in clinical testing, the accuracy of diagnostics can be significantly impacted by environmental instabilities such as pH variations, temperature fluctuations, ionic concentration changes, and interfacial effects. This is particularly problematic when traditional Poisson distribution algorithms are applied to detections at extremely low concentrations. At such low levels, the inherent randomness and uncertainty, compounded by background noise, including interference signals caused by nonspecific adsorption, result in considerable detection errors that are unacceptable in clinical contexts.

In some forms, the methods enable comprehensive analysis of entire samples by the principle of exhaustive testing. In some forms, the methods employ a computational method that is insensitive to both systematic and random errors, termed the “Adaptive Differential Method” that leverages control samples with known concentrations to facilitate rapid initial testing without the need for generating standard curves, allowing for precise differential calculations of sample concentrations. As the number of known concentration tests in the control samples increases, more data points are collected, enabling continual refinement and enhancement of the model. The method includes an iterative process, involving the re-fitting of curves with new data points to generate updated predictive functions, heightens the model's accuracy for unknown data predictions, utilizing incremental learning within the domain of machine learning.

In some forms, the differential adaptive algorithm involves subtracting the noise signal from a control blank sample to obtain concentration differences. The comprehensive washing method uses a known concentration control sample, washing until the control sample signal meets the error range of standard curve value, ensuring the test area signal is valid.

In some forms, the methods include an algorithm to reduce errors in a Spatial Reflection Symmetry Microfluidic Biochip, especially regarding nonspecific adsorption issues at low concentrations. In some forms, the methods include a mathematical model based on Poisson distribution for eliminating errors caused by nonspecific adsorption in microfluidic chip assays for single-molecule protein detection. In some forms, the methods include the steps of:
(a) Selecting the fluorescent signals from both the fluid sample and the control sample to follow Poisson distributions. The Poisson distribution is suitable for describing sparse events such as signals from single-molecule protein detection;
(b) Selecting the background noise due to nonspecific adsorption to follow an independent Poisson distribution; and
(c) Selecting models to represent the Poisson distributions of signals from the fluid sample and the control sample.

### 2. Exemplary Optimization Parameters

In some forms, the methods optimize one or more of the parameters including the numerical aperture (NA) of the objective lens, exposure time, and detector sensitivity, particularly focusing on the detection limits of the CMOS sensor used in commercial fluorescence microscopy.

In some forms, the methods determine photon emission rate (Rₑₘ) of quantum dots (QDs) on beads by the photon absorption rate (R_{abs}) and the quantum yield (QY):

$${R}_{em} = QY \times {R}_{abs}$$

In some forms, the methods calculate photon emission rate (Rₑₘ) from a single QD using:

$${R}_{em} = QY \times \frac{{σ}_{abs} \times {I}_{0}}{{hv}_{exc}}$$

Where:
σ_{abs} is the absorption cross-section of QDs (1×10⁻¹⁶ cm², the date from the datasheet provided by manufacturer DiagNano™, typically measured through experimental measurements of the QDs' absorption properties at the specific wavelength of 550 nm)
I₀ is the excitation light intensity (1×10⁵ W/cm²), equal to the ratio between the power of a laser beam (10 mW) and the area of the light spot;
hv_{exc} is the individual photon energy at the excitation wavelength;
h is Planck constant (6.626×10⁻³⁴ J·s); and
vₑₓₑ is the photon frequency (5.45×10¹⁴ Hz), which is equal to the ratio between the velocity of light and excitation wavelength.

For silica beads, which are highly transparent and exhibit minimal quenching, the calculated photon emission rate (R_{em,silica}) is approximately 2.35×10⁷ photons/s.

$$e {R}_{em , magnatic} = {R}_{abs} \times QY \times QF \times {A}_{exc}$$

### Where:

QF is a quenching factor, represents a reduction in quantum yield due to energy transfer processes or other quenching mechanisms caused by the proximity of the magnetic material; and
A_{exc} is attenuation of excitation light due to absorption/scattering by the magnetic bead.

Consequently, photon emission rate of magnetic beads (R_{em,magnatic}) is reduced to about 5.89×10⁶ photons/s, which is used to determine the number of photons detected (N_{detected}):

$${N}_{detected} = {R}_{em} \times {A}_{em} \times T \times {η}_{detector} \times τ \times {f}_{collection}$$

### Where:

Aₑₘ is the emission attenuation factor, which represents the reduction in emitted fluorescence intensity due to absorption and scattering of emitted photons by the bead material before they reach the detector;
T is the overall transmission efficiency of the optical system, accounting for losses due to optical components (50% or 0.5, based on typical losses in the optical components used e.g., lenses, filters);
η_{detector} is the quantum efficiency of the detector (CMOS sensor) at the emission wavelength of 611 nm (50% or 0.5, based on the specifications of detector provided by the microscope manufacturer);
τ is the exposure time (1 second, chosen based on the requirements of the experimental design and the need to balance signal acquisition with imaging speed); and
f_{collection} is the fraction of emitted photons collected by the objective lens, depending on its numerical aperture NA of the lens:

$${f}_{collection} = \frac{1 - \sqrt{1 - {NA}^{2}}}{2} .$$

The minimum number of detected photons (N_{detected}) required to achieve the signal-to-noise ratio (SNR) of 100 is approximately 10,025 photons. This is determined using the SNR definition formula:

$$S N R = \frac{{N}_{detected}}{\sqrt{{N}_{detected + {σ}_{read}^{2}}}}$$

### Where:

σ_{read} is the read noise of the detector in electrons rms. (σ_{read}=5 rms, obtained from the technical specifications of microscope CMOS detector).

To achieve the required N_{detected}, the minimum fraction of collected photons (f_{collection}) must satisfy:

$${f}_{collection} \geq \frac{{N}_{detected}}{{R}_{em} \times T \times {η}_{{detector}^{\times τ}}} .$$

The minimum NA is then calculated using:

NA≥\sqrt{2×f_{collection}}. The lower NA requirements for silica beads facilitate large field-of-view imaging, as low-magnification objectives with wide fields can be used without compromising detection sensitivity. This advantage is crucial for applications requiring the analysis of large populations of beads or widespread distribution of targets. The BiZi-FIA platform, employing silica beads as the primary capture substrate, demonstrates a marked improvement in signal-to-noise ratio (SNR) compared to traditional magnetic bead-based assays.

E. Adaptive Differential Noise Correction with BiZi-FIA for Broad Dynamic Range Rapid Precision Detection

In some forms, the methods implement an adaptive Differential Noise Correction (ADNC) algorithm to enhance the precision and reliability of the Bilateral Zigzag Flow Immunoassay (BiZi-FIA) system in detecting ultra-low concentration proteins.

In some forms, the methods leverage the symmetrical bilateral assay setup of BiZi-FIA, where test and control samples are introduced from opposite sides of the flow channel, ensuring similar environmental exposure for both samples to accurately distinguishing true signal from background noise. Methods for implementation of ADNC involve several key steps, including:
(i) data acquisition,
(ii) differential analysis,
(iii) noise modeling, and
(iv) iterative optimization using gradient descent.

The mathematical framework for the ADNC algorithm is grounded in the Poisson distribution, which is suitable for describing sparse events like single-molecule protein detection signals. Fluorescent signals from the test sample (Sₜ) and the control sample (S_{c}) are modeled as independent Poisson-distributed variables. Background noise (S₀), a component of both the test and control signals, is also assumed as Poisson-distributed. Importantly, the control sample (S_{c}) contains background noise but may also include signal contributions from known concentrations of a standard. To refine detection accuracy, the algorithm calculates the gradient of the signal difference between test and control samples using:

$${f}^{′} ( Tc ) = ( {S}_{T} - {S}_{c} ) / ( x - C )$$

where x and C are the concentrations of the test and control samples, respectively. This differential analysis accounts for nonspecific adsorption and isolates the true signal.

Systematic experiments are conducted across various known concentrations to train the ADNC algorithm through differential analysis. Data from bilateral assays is collected to establish relationships between test and control areas. Test samples highlight experimental variations at specific concentrations, forming the basis for gradient analysis, which normally caused by “pH”: pH variations, “T”: temperature fluctuations, “k_{off}”: dissociation constant of specific antibody, “γ”: interfacial energy, and “I”: ionic concentration changes. “Nᵤₛₑ”: number of microbeads used, “Nₐₙₗ”: number of microbeads analyzed.

During the training process, the algorithm learns to adjust test readings based on control responses. The software has an initial setup, which involves inputting the concentration values of quantitation sample (Q) and control samples (C). The process begins with a bilateral assay of the test sample against the quantitation sample to obtain the first test signal (Sₜ₁) and the quantitation signal (S_{Q}). This is followed by a bilateral assay with a control sample (C) to get the second test signal (Sₜ₂) and the control signal (S_{C}). Finally, a bilateral assay with a blank sample provides the third test signal (Sₜ₃) and the background signal (S₀). After repeating the bilateral assays, the software records the signal gradients f′(CQ), f′(C0), and f′(Q0) for the specific biomarker as follows.

$${f}^{′} ( CQ ) = \frac{{S}_{c} - {S}_{Q}}{C - Q}$$

$${f}^{′} ( C 0 ) = \frac{{S}_{c} - {S}_{0}}{C}$$

$${f}^{′} ( Q 0 ) = \frac{{S}_{Q} - {S}_{0}}{Q}$$

During the initial setup, software will iterate through the threshold range to ensure that

$${f}^{′} ( CQ ) \geq {f}^{′} ( C 0 ) \geq {f}^{′} ( Q 0 )$$

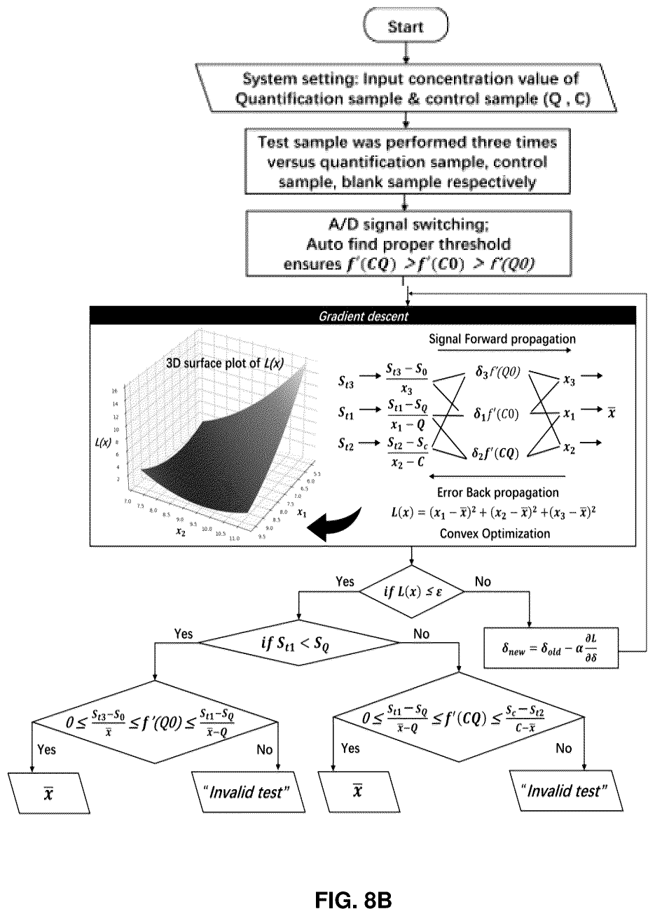

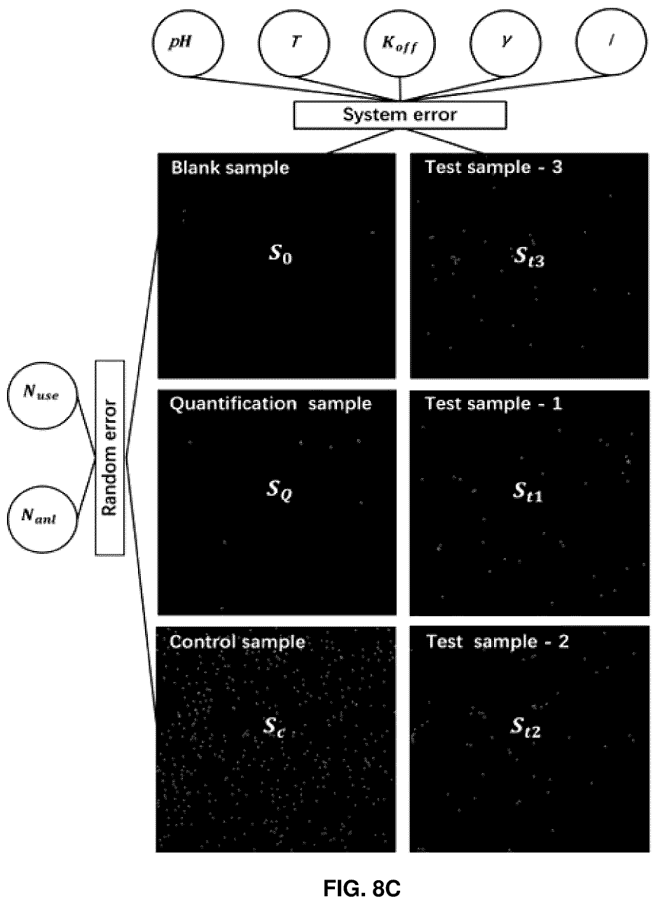

which can be understood in the context of Lagrange's differential meaning value theorem, explaining the underlying connection between the average rate of change in the function and its instantaneous rates at different points (FIGS. 8A-8C).

By calculating the mean difference in signal intensity between consecutive points in the assay field, the algorithm can fine-tune its correction factor, δ, to address local variations caused by measure error. Based on the available concentrations (C, Q) and the corresponding fluorescence detection signals, we establish functions for x in terms of δ.

$${x}_{1} = Q + \frac{{S}_{t 1} - {S}_{Q}}{{δ}_{1} {f}^{′} ( CQ )}$$

$${x}_{2} = C + \frac{{S}_{t 2} - {S}_{c}}{{δ}_{2} {f}^{′} ( C 0 )}$$

$${x}_{3} = \frac{{S}_{t 3} - {S}_{0}}{{δ}_{3} {df}^{′} ( Q 0 )}$$

Next, validation is performed using cross-validation techniques to assess the accuracy of the model. Parameters are fine-tuned through gradient descent optimization, minimizing variance among concentration estimates across repeated tests. The loss function, L(x, x̅), used in the optimization process is defined as:

$$L ( x , \overline{x} ) = {\sum }_{i = 1}^{n} {( {x}_{i} - \overline{x} )}^{2}$$

where xᵢ represents individual concentration estimates, and x̅ is their mean value. The correction factor δ is iteratively updated using:

$${δ}_{new} = {δ}_{old} - α \frac{\partial L}{\partial δ} .$$

This iterative process ensures convergence to an optimal correction factor that minimizes discrepancies and gradually reduces the loss, helping it find the optimal model parameters δ₁, δ₂, δ₃. This process involves calculating the partial derivatives of the loss function L(x, x̅) concerning δ and iteratively updating the values of δ based on these derivatives, until the L(x, x̅)≤ε. δ_{old} refers to the current value of the parameter from the previous iteration. ∝L/∝δ is the gradient of the loss function with respect to the parameter, indicating how the loss function changes with small changes in δ. α is the learning rate that determines the size of each step taken during the update. δ_{new} is the updated parameter value used in the next iteration. Typically, “1” is used as the initial value of δ, starting with the ideal state. Using the optimized correction factor (δ1, δ2, δ3), the program calculates the values of the unknowns x₁, x₂, x₃. Finally, the program outputs precise concentration by taking the mean value, x̅ (FIG. 8B). After three repeated bilateral assays, a standard curve function, f(x), with the optimized signal gradient f′(x) is established for specific biomarkers, allowing followed sample tests with a bilateral assay to calculate the concentration quickly. The software automatically verifies the accuracy of the concentration output results. If it detects values exceeding areasonable concentration range, the system will output “invalid data” instead of generating erroneous data. By following these steps, the ADNC model can help understand and correct the errors caused by environmental variables for single-molecule protein detection, thereby improving the accuracy and reliability of the detection.

The disclosed compositions and methods can be further understood through the following numbered paragraphs.

1. A microfluidic chip comprising:
a microfluidic platform comprising one or more microfluidic flow paths, wherein at least one of the microfluidic flow paths comprises an inlet conduit, a flow-trap junction (FTJ) structure, and an outlet conduit, wherein the microfluidic flow path is configured for movement of fluid from the inlet conduit into the FTJ structure, and from the FTJ structure into the outlet conduit,
wherein the inlet conduit is wider where the fluid moves from the inlet conduit into the FTJ structure than where the fluid is introduced into the inlet conduit, wherein the inlet conduit contains a multiplicity of hydrophobic micropillar structures,
wherein the FTJ structure comprises a flow layer and a trap layer, wherein the flow layer is in contact with, on top of, and overlapping with the trap layer,
wherein the flow layer comprises a plurality of flow microfluidic channels each comprising a top, side walls, and an opening on the bottom, wherein the surfaces of the flow microfluidic channels are hydrophobic, wherein the flow microfluidic channels allow free passage of micro-scale particles and nano-scale objects, wherein the fluid flows in the same direction in all of the flow microfluidic channels,
wherein the flow microfluidic channels are parallel to each other, wherein the trap layer comprises a plurality of trap microfluidic channels each comprising a bottom, side walls, and an opening on the top, wherein the trap microfluidic channels are parallel to each other, wherein the surfaces of the trap microfluidic channels are hydrophilic, wherein the fluid flows in the same direction in all of the trap microfluidic channels,
wherein the flow microfluidic channels are not parallel to the trap microfluidic channels, wherein the flow microfluidic channels and the trap microfluidic channels allow fluid movement from the flow microfluidic channels into the trap microfluidic channels via the openings on the bottom and openings on the top, respectively,
wherein the trap microfluidic channels, the transition from the flow microfluidic channels to the trap microfluidic channels, or a combination of both the trap microfluidic channels and the transition from the flow microfluidic channels to the trap microfluidic channels are configured to allow passage of the nano-scale objects through the trap microfluidic channels, whereby the passaged nano-scale objects flow into the outlet conduit,
wherein the trap microfluidic channels, the transition from the flow microfluidic channels to the trap microfluidic channels, or a combination of both the trap microfluidic channels and the transition from the flow microfluidic channels to the trap microfluidic channels are configured to trap the micro-scale particles in the trap microfluidic channels, whereby the trapped micro-scale particles, in combination, form an array within the FTJ structure.

2. The chip of paragraph 1, wherein the side walls of the trap microfluidic channels are straight and parallel to each other, wherein the height of the trap microfluidic channels is less than the diameter of the micro-scale particles.

3. The chip of paragraph 2, wherein the height of the trap microfluidic channels is more than the diameter or long dimension of the nano-scale objects.

4. The chip of paragraph 1, wherein the side walls of the trap microfluidic channels are not straight such that the trap microfluidic channels vary in width in a regular pattern along their lengths, wherein the pattern of width variation forms narrowings in the width of the trap microfluidic channels, wherein the width of the narrowings are less than the diameter of the micro-scale particles, wherein the width of the narrowings are more than the diameter or long dimension of the nano-scale objects.

5. The chip of paragraph 4, wherein all or a subset of the narrowings overlap the flow layer between some or each of the openings on the bottoms of adjacent flow microfluidic channels.

6. The chip of paragraph 4 or 5, wherein all or a subset of the narrowings overlap the opening on the bottom of some or each of the flow microfluidic channels.

7. The chip of any one of paragraphs 4 to 6, wherein a subset of the narrowings overlap the opening on the bottom of some or each of the flow microfluidic channels and a subset of the narrowings overlap the flow layer between some or each of the openings on the bottoms of adjacent flow microfluidic channels.

8. The chip of any one of paragraphs 4 to 6, wherein a subset of the narrowings overlap the opening on the bottom of each of the flow microfluidic channels and a subset of the narrowings overlap the flow layer between each of the openings on the bottoms of adjacent flow microfluidic channels.

9. The chip of any one of paragraphs 6 to 8, wherein the narrowings overlapping the openings on the bottoms of the flow microfluidic channels form a small trap entrance on the down-flow side of the opening and a large trap entrance on the down-flow side of the opening for alternating trap microfluidic channels, wherein the size of the small trap entrance is less than the diameter of the micro-scale particles, wherein the size of the small trap entrance is more than the diameter or long dimension of the nano-scale objects, wherein the size of the large trap entrance is more than the diameter of the micro-scale particles.

10. The chip of any one of paragraphs 1 to 3, wherein the flow microfluidic channels and the trap microfluidic channels are at a right angle to each other.

11. The chip of any one of paragraphs 1 to 3, wherein the flow microfluidic channels and the trap microfluidic channels are at an oblique angle to each other.

12. The chip of any one of paragraphs 1 to 3, wherein the flow microfluidic channels and the trap microfluidic channels are at an angle of between 60° to 90°, between 60° to 90°, between 60° to 90°, between 60° to 90°, between 60° to 90°, between 70° to 90°, between 80° to 90°, between 85° to 90°, between 87° to 90°, between 88° to 90°, or between 89° to 90°, to each other.

13. The chip of any one of paragraphs 1 to 12, wherein the side walls of the trap microfluidic channels are angled toward the up-flow ends of the flow microfluidic channels.

14. The chip of any one of paragraphs 1 to 13, wherein the microfluidic flow path further comprises a sample inlet, wherein the microfluidic flow path is configured for movement of fluid from the sample inlet into the inlet conduit.

15. The chip of any one of paragraphs 1 to 14, wherein the microfluidic flow path further comprises a plurality of outlet channels, wherein the microfluidic flow path is configured for movement of fluid from the trap microfluidic channels into the outlet channels and from the outlet channels into the outlet conduit.

16. The chip of paragraph 15, wherein each trap microfluidic channel is flowably connected to a different one of the outlet channels.

17. The chip of any one of paragraphs 1 to 14, wherein the flow layer of the FTJ structure further comprises a plurality of outlet channels, wherein the outlet channels are interspersed between and parallel to the flow microfluidic channels, wherein the outlet channels each comprise a top, side walls, and an opening on the bottom, wherein the outlet channels and the trap microfluidic channels allow fluid movement from the trap microfluidic channels into the outlet channels via the opening on the bottom and openings on the top, respectively, wherein the microfluidic flow path is configured for movement of fluid from the trap microfluidic channels into the outlet channels and from the outlet channels into the outlet conduit.

18. The chip of paragraph 17, wherein the outlet channels alternate with the flow microfluidic channels in the flow layer of the FTJ structure.

19. The chip of any one of paragraphs 1 to 14, wherein the flow layer of the FTJ structure further comprises an outlet channel, wherein the outlet channel comprises a top, side walls, and an opening on the bottom, wherein the outlet channel overlaps the down-flow ends of the trap microfluidic channels, wherein the outlet channel and the trap microfluidic channels allow fluid movement from the trap microfluidic channels into the outlet channel via the opening on the bottom and openings on the top, respectively, wherein the microfluidic flow path is configured for movement of fluid from the trap microfluidic channels into the outlet channel and from the outlet channel into the outlet conduit.

20. The chip of any one of paragraphs 1 to 19, wherein the chip is transparent in one or more regions, wherein the chip is transparent in a region corresponding to the array.

21. The chip of any one of paragraphs 1 to 20, wherein the array comprises a surface area of 25 mm² or less.

22. The chip of any one of paragraphs 1 to 21, wherein the microfluidic platform comprises two microfluidic flow paths.

23. The chip of paragraph 22, wherein the two microfluidic flow paths are flowably connected to a single fluid reservoir, wherein the fluid reservoir is flowably connected to the respective inlet conduits of the two microfluidic flow paths.

24. The chip of paragraph 22 or 23, wherein the two microfluidic flow paths are symmetrically disposed on the microfluidic platform.

25. The chip of any one of paragraphs 22 to 24, wherein the two microfluidic flow paths are symmetrically disposed on the chip.

26. The chip of any one of paragraphs 1 to 25 further comprising a plurality of micro-scale particles.

27. The chip of paragraph 26, wherein the micro-scale particle comprise a microbead.

28. The chip of paragraph 27, wherein the microbead comprises a magnetic microbead or a silica microbead, optionally wherein the silica microbead comprises a diameter of about 2 μm to about 56 μm, inclusive, or about 2.97 μm to about 3.0 μm.

29. The chip of any one of paragraphs 26 to 28, wherein the micro-scale particles further comprise a first capture agent specific for a target biomarker.

30. The chip of paragraph 29, wherein the first capture agent is conjugated to the micro-scale particle via streptavidin.

31. The chip of paragraph 29 or 30, wherein one or more of the first capture agents are bound to the target biomarker.

32. The chip of any one of paragraphs 26 to 31, wherein the micro-scale particles have a diameter of between about 1 μm and about 5 μm, inclusive.

33. The chip of any one of paragraphs 29 to 32, wherein each of the micro-scale particles comprise between about 200,000 and about 400,000 first capture agents, inclusive.

34. The chip of any one of paragraphs 29 to 33, wherein the first capture agent is selected from the group consisting of a nucleic acid, a protein, a polypeptide, a lipid, a carbohydrate, and a small molecule, optionally wherein the protein is an antibody.

35. The chip of any one of paragraphs 29 to 34, wherein the first capture agent comprises DNA or RNA, or both.

36. The chip of any one of paragraphs 26 to 35, wherein the micro-scale particles are present within the array.

37. The chip of paragraph 36, wherein the micro-scale particles are present within the array at a density of about 100 micro-scale particles/μm².

38. The chip of any one of paragraphs 26 to 37, wherein the array comprises from about 1×10⁴ micro-scale particles, to about 1×10⁶ micro-scale particles, inclusive, optionally about 4×10⁵ micro-scale particles.

39. The chip of any one of paragraphs 1 to 38, wherein the array has an area of from about 10 mm² to about 50 mm², inclusive, optionally about 25 mm².

40. The chip of any one of paragraphs 1 to 39 further comprising a plurality of nano-scale objects.

41. The chip of paragraph 40, wherein the nano-scale objects comprise a second capture agent specific for the target biomarker.

42. The chip of paragraph 41, wherein one of the second capture agents is bound to one or more of the target biomarkers bound to the first capture agents.

43. The chip of paragraph 41 or 42, wherein the nano-scale objects further comprise a reporter molecule.

44. The chip of paragraph 43, wherein the reporter molecule comprises a highly bright quantum dot nanoparticle.

45. The chip of any one of paragraphs 1 to 44, wherein the micropillars span the height of the inlet conduit.

46. The chip of any one of paragraphs 1 to 45, wherein the trap microfluidic channels comprise a hydrophilic polymer.

47. The chip of paragraph 46, wherein the hydrophilic polymer comprises polyethylene glycol (PEG).

48. A method for detecting a target biomarker in a fluid sample, the method comprising:
(a) introducing the fluid sample to one or more of the microfluidic flow paths of the microfluidic chip of any one of paragraphs 1 to 25, wherein the fluid sample comprises, or is bought into contact with after its introduction, a plurality of micro-scale particles and a plurality of nano-scale objects, and
(b) performing digital chromatography on the chip, wherein the digital chromatography identifies the presence and/or quantity of the target biomarker in the fluid sample.

49. The method of paragraph 48, wherein step (a) further comprises introducing to a different microfluidic flow path of the same chip a control sample comprising a known amount of the target biomarker.

50. The method of any one of paragraphs 48 or 49, wherein the performance of digital chromatography of step (b) comprises actuating movement of fluid through the microfluidic flow paths in the microfluidic chip, wherein the movement filters and washes the micro-scale particles within the FTJ structure.

51. The method of paragraph 50, wherein the filtering of the micro-scale particles in the FTJ structure traps, and forms an array of, the micro-scale particles within the FTJ structure.

52. The method of paragraph 51, wherein step (b) further comprises imaging the array of micro-scale particles within the microfluidic chip.

53. The method of any one of paragraphs 48 to 52, wherein the micro-scale particles comprises a microbead.

54. The method of paragraph 53, wherein the microbead comprises a magnetic microbead.

55. The method of any one of paragraphs 48 to 54, wherein the micro-scale particles further comprise a first capture agent specific for a target biomarker.

56. The method of paragraph 55, wherein the first capture agent is conjugated to the micro-scale particle via streptavidin.

57. The method of paragraph 55 or 56, wherein one or more of the first capture agents are bound to the target biomarker.

58. The method of any one of paragraphs 48 to 57, wherein the nano-scale objects comprise a second capture agent specific for the target biomarker.

59. The method of paragraph 58, wherein one of the second capture agents are bound to one or more of the target biomarkers bound to the first capture agents.

60. The method of paragraph 58 or 59, wherein the nano-scale objects further comprise a reporter molecule.

61. The method of paragraph 60, wherein the reporter molecule comprises a highly bright quantum dot nanoparticle.

62. The method of any one of paragraphs 57 to 61, wherein step (b) further comprises detecting and measuring the target biomarkers bound to the first capture agents on the micro-scale particles within the array.

63. The method of any one of paragraphs 55 to 62 further comprising, prior to step (a),
(i) incubating the fluid sample with the micro-scale particles for a time and in an amount effective for binding of the target biomarkers to the first capture agent; and
(ii) optionally washing the micro-scale particles.

64. The method of paragraph 63 further comprising, prior to step (a), contacting the micro-scale particles with the nano-scale objects for a time and in an amount effective for binding of the target biomarkers to the second capture agent.

65. The method of any one of paragraphs 48 to 64, wherein steps (a) and/or (b) comprise a total time of between about 10 and 1000 about seconds, inclusive, optionally about 180 seconds.

66. The method of any one of paragraphs 48 to 65, wherein the method detects at least 90%, such as 91%, 92%, 93%, 94%, 95%, 96%, 97%, 98%, 99%, or 100% of the biomarkers within the fluid sample.

67. The method of any one of paragraphs 48 to 66, wherein the fluid sample comprises a biomarker at a concentration of about 1 IU/mL.

68. The method of any one of paragraphs 48 to 67, wherein the fluid sample comprises a biomarker at a concentration of about 10⁻¹⁸ M to about 10⁻⁵ M, inclusive.

69. The method of any one of paragraphs 48 to 68, wherein the fluid sample comprises a volume of between about 1.0 μL to about 100 μL, inclusive, optionally about 50 μL.

70. The method of any one of paragraphs 48 to 69, wherein the sample comprises between one and 100 different biomarkers, optionally between one and 10 different species of biomarkers.

71. The method of any one of paragraphs 48 to 70, wherein one biomarker comprises an immunoglobulin.

72. The method of any one of paragraphs 48 to 71, wherein one biomarker comprises IgE.

73. The method of any one of paragraphs 48 to 72, wherein one biomarker comprises an interferon.

74. The method of any one of paragraphs 48 to 73, wherein one biomarker comprises TNF-alpha.

75. The method of any one of paragraphs 48 to 74, wherein one biomarker comprises a tumor antigen.

76. The method of paragraph 75, wherein the tumor antigen is selected from the group consisting of Prostate-Specific Antigen (PSA), Carcinoembryonic Antigen (CEA), Alpha-Fetoprotein (AFP), Cancer Antigen 125 (CA-125), Cancer Antigen 19-9 (CA 19-9), Human Epidermal Growth Factor Receptor 2 (HER2/neu), Cytokeratin 19 Fragment (CYFRA 21-1), B-Raf Proto-Oncogene, and Epidermal Growth Factor Receptor (EGFR).

77. The method of any one of paragraphs 48 to 73, wherein one biomarker comprises a biomarker derived from a pathogen.

78. The method of paragraph 77, wherein the pathogen is selected from the group consisting of a bacterium, a fungus, a virus and a protozoan.

79. The method of paragraph 78, wherein the virus is a coronavirus, optionally wherein the coronavirus is SARS-Cov-2.

80. The method of any one of paragraphs 48 to 79, wherein the target biomarker within the fluid sample is derived from a bodily fluid from a subject.

81. The method of paragraph 80, wherein the bodily fluid is selected from the group consisting of blood, sweat, semen, serum, bile, saliva, tear fluid, pus, mucus, pleural fluid, vitreous fluid, spinal fluid, synovial fluid, amniotic fluid and urine.

82. The method of paragraph 81, wherein the bodily fluid is tear fluid.

The present disclosure will be further understood by reference to the following non-limiting examples.

### EXAMPLES

### Example 1: Design and Fabrication of a BiZi-FIA Microfluidic Chip

A device based on BiZi-FIA was designed to isolate and precisely detect single molecules of biomarkers at a low sample volume (FIGS. 1A-1E).

### Methods

Sylgard 184 Silicone Elastomer Kit was purchased from Dow Corning, USA.

Superparamagnetic beads covalently binding carboxylic acid and surface epoxy groups were purchased from Thermo Fisher Scientific, USA (DYNABEADS® M-270) and used without any further treatment. Artificial tears (A7720) were purchased from Solarbio, China.

A microfluidic device for BiZi-FIA was designed and fabricated using conventional photolithography and soft lithography techniques. The fabrication process began with the careful design of the flow/trap layer mask, which was specifically tailored to define the flow conduits and trap structures important for the Filter-like Flow and Trap Junction. The mask was then materialized using lithography on an apt substrate.

For the flow layer, MicroChem's SU-8 2025 photoresist was utilized, renowned for its capacity to achieve a 5.5 μm thickness. The substrate underwent spin-coating at 4000 rpm for 30 seconds, succeeded by a soft bake at 65° C. for 5 minutes. The flow/trap layer mask was then meticulously aligned and subjected to UV light exposure with a strength of 100 mJ/cm². After that, the layer mask conducted a post-bake at 95° C. for 5 minutes and developed with the MicroChem SU-8 Developer for 2-3 minutes. The regions that remained unexposed were washed, culminating in the manifestation of the envisaged flow conduit patterns.

A mold for the trap layer was prepared by employing MicroChem's SU-8 3005, specifically chosen for its proficiency in producing a structure with a 3 μm height.

A 45-degree angle photolithography technique was harnessed, wherein the flow/trap layer mask was aligned with precision at this angle, ensuring the UV light exposure was executed at the important 45-degree angle. Following a soft bake at 65° C. for 2 minutes, UV exposure was conducted at approximately 90 mJ/cm². A subsequent post-exposure bake was performed at 95° C. for 3 minutes. The developed resist then served as a foundational mold for the trap layer.

Sylgard 184 Silicone Elastomer Kit (Dow Corning, USA) was selected and mixed the base and curing agent in a 10:1 ratio for preparing the trap layer. After the meticulously mixing and subsequent degassing, this mixture was then cast onto the mold and underwent a curing process at 65° C. for 4-6 hours. Post-curing, the PDMS structure was cautiously demolded, ensuring alignment with the photolithography's incident angle, unveiling the intricate trap patterns.

To ensure the bonding was robust and enduring, trap and flow layers were treated with plasma and precisely aligned before bonding. To augment the trap layer's biocompatibility and mitigate non-specific binding, a surface modification was undertaken using Sigma-Aldrich's PEG 2000. A PEG solution was judiciously introduced through the trap layer's outlet port, with the flow layer's inlet port securely sealed. This strategic approach ensured that the PEG solution was confined to the trap layer. Following the 8-hour incubation period, the device was rinsed to eliminate residual PEG.

### Chip Fabrication

Lithography was used to ablate a simple microstructure pattern on a silicon wafer.

Mix PDMS and a hardener in a 10:1 weight ratio and degas the mixture in a vacuum to remove air bubbles.

Pour the PDMS mixture onto the ablated silicon wafer and cure at 70° C. for 30 minutes.

After curing, peel off the PDMS layer from the silicon wafer to form a PDMS convex mold.

Treat the PDMS mold with O2 plasma for 1 minute and then immerse it in a 0.1 M PEG solution (average molecular weight 6000 g/mol) to form an isolation film.

Repeat the PDMS pouring and curing steps on the convex mold to create a concave PDMS mold.

Finally, bond the PDMS mold to a blank PDMS substrate to form the microfluidic chip.

### Surface Treatment

Preparation of Surface Treatment Reagent: Prepare a 0.1 M solution of Polyethylene Glycol (PEG) with an average molecular weight of 6000 g/mol. This solution serves as the surface treatment reagent.

Setting Up the Surface Treatment System: Arrange a surface treatment pool connected to the inlet of the surface treatment channels in the microfluidic chip. Ensure the system is sealed and leak-proof.

Connect a precision pump to the surface treatment pool to control the flow of the PEG solution.

Initiating the Surface Treatment Process: Activate the precision pump to introduce the PEG solution into the surface treatment channels. The flow rate should be maintained at a consistent and controlled pace to ensure even distribution of the reagent.

As shown in FIGS. 1A-1E, 2A-2I and 3A-3I, the PEG solution simultaneously flows through the array capture channels, which are parallel to the surface treatment channels. This ensures uniform exposure of the channels to the treatment reagent.

As depicted in FIG. 2E, reverse-oriented channels positioned at the terminus of each flow channel create turbine valve structures, with centrally located valves in the flow layer inducing controlled hydrodynamic resistance to direct fluid laterally into the trap network. The system facilitates selective bead capture through an adaptive zigzag pathway dynamically governed by trap occupancy states: unoccupied traps exhibit minimal resistance, enabling bead ingress and settling, whereas occupied traps generate elevated resistance profiles, redirecting fluid recirculation to primary channels and steering subsequent beads toward adjacent available sites. This self-regulating process ensures spatially ordered bead deposition, as fluid dynamics autonomously adjust to real-time trap availability. Upon saturation of all trapping sites, surplus beads undergo controlled evacuation via the terminal flow conduit, preventing overcrowding while maintaining optimal particle density. The architecture operates through autonomous fluidic regulation, leveraging occupancy-dependent resistance modulation, self-directed particle routing, and systemic overfill prevention to achieve non-mechanical flow control, self-limiting deposition, and scalable array formation without reliance on external control systems or active monitoring components.

Maintaining Flow and Treatment Time: Maintain a constant flow rate of the PEG solution throughout the treatment process. Recommended flow rate parameters can be around 0.5 mL/min, but this may vary based on the specific design and dimensions of the microfluidic chip.

The duration of the surface treatment process should be carefully timed. A typical treatment time can range from 25 to 30 minutes. This duration ensures adequate surface modification without overexposure.

Completion and Discharge of Reagent: After the treatment duration is completed, gradually decrease the flow rate and eventually stop the pump.

Allow the PEG solution to be completely discharged from the exit port of the microfluidic chip. Ensure that no residual treatment reagent remains within the channels.

Post-Treatment Handling: Rinse the surface-treated channels with deionized water to remove any unbound PEG molecules.

Dry the microfluidic chip under a gentle stream of nitrogen to remove any remaining moisture.

Verification of Surface Treatment: Assess the success of the surface treatment by measuring the contact angle of a water droplet on the treated surface. A significant decrease in contact angle indicates successful hydrophilic modification.

Optionally, use additional analytical techniques such as SEM (Scanning Electron Microscopy) or FTIR (Fourier Transform Infrared Spectroscopy) for further verification and analysis of the surface characteristics.

Use a CCD camera to measure the contact angle of a 5 μL droplet of deionized water on the differently treated PDMS substrates.

Evaluate the hydrophilic property of the treated surface by recording the contact angle over up to 420 hours for the plasma-PEG treated surface.

Examine the morphology of the PDMS before and after surface treatment using a SEM microscope.

Employ FTIR (Fourier Transform Infrared Spectroscopy) to compare the PDMS samples before and after treatment.

This detailed surface treatment procedure aims to enhance the hydrophilicity of the PDMS surfaces in the microfluidic chip, thereby minimizing nonspecific adsorption and improving chip performance for various applications.

Detection Antibody Coupling with Ultrabright Fluorescent Label

To prepare the detection antibody coupled with streptavidin-functionalized quantum dot nanoparticle, the standardized protocol as reported in the literature was followed.

Initially, a working solution of streptavidin quantum dot nanoparticles with a 625 nm emission peak was prepared according to the manufacturer's instructions (catalog number of DNQ-N008, DiagNano™, CD Bioparticles). The prepared bead solution (50 μL) was centrifuged at 5000 rpm for 2 minutes at room temperature to remove the supernatant.

Following this, the beads underwent a thorough washing process in which they were gently vortexed and then centrifuged three times using 1 mL of phosphate-buffered saline (PBS).

Subsequently, 25 μg of the antibody solution was introduced to the washed beads. The tube containing the mixture was gently pipetted to ensure uniform mixing. The tube was then incubated at room temperature with gentle shaking on an orbital shaker for 1 hour, allowing sufficient time for the antibodies to bind effectively to the streptavidin-coated Qt nanoparticles. Once the incubation period was completed, the supernatant was removed after centrifugation.

After that, the beads were subjected to three additional washes using 1 mL of PBS each time to remove any unbound antibodies from the beads. After the final wash, the antibody-coupled Qt nanoparticles were resuspended in 100 μL of PBS for immediate use or in a suitable storage buffer for longer-term storage.

In conclusion, the antibody-coupled quantum dot nanoparticles were primed and ready for integration into further experimental applications, such as immunofluorescence assays or protein detection.

### Coupling Capture Antibody on Beads

A method was elucidated for covalently immobilizing antibodies onto the surface of Dynabeads® M-270 Epoxy. It's imperative to note that the protocol described herein was designed and executed strictly in accordance with the guidelines provided in the Antibody Coupling Kit from Thermo Fisher Scientific, Dynabeads (3 μm diameter carboxylic acid and epoxy-linked superparamagnetic beads).

Initially, a series of buffers and solutions, including C1 for bead washing and preparation, C2 as an activator for coupling, LB as a low-salt buffer for non-specific binding removal, HB as a high-salt buffer to enhance binding, and SB for long-term bead storage, were prepared by Antibody Coupling Kit. These reagents, of analytical grade, were confirmed to be compatible with protease and phosphatase inhibitors and were stored at 2-25° C.

For a 5 mg bead preparation, exactly 5 mg of Dynabeads® M-270 Epoxy was weighed out with beads at room temperature to prevent condensation. The beads were then washed using the C1 solution to ensure their surface was ready for coupling. After magnetic separation, a volume of antibody was added, and the remaining volume to reach 250 μl was filled with C1. An additional 250 μl of the C2 activator solution was introduced, facilitating the covalent attachment of the antibodies to the bead surface. The mixture was then incubated on a roller at 37° C. overnight.

The next day, the tube was placed on a magnet, and the supernatant was discarded. The beads underwent sequential washes with HB and LB to remove non-specifically bound substances and enhance specific binding. A short SB wash was conducted, followed by a long SB wash where the beads were incubated at room temperature for 15 minutes. The beads were finally resuspended in 500 μl of SB, ensuring their stability and longevity. The final bead concentration is 10 mg antibody coupled beads/ml.

### Results

The designed unidirectional microcapillary flow allows simultaneous transportation and immobilization of many microbeads in a chip. The innovation of this device is portable and low-cost because the device does not need to connect with syringe pumps and other additional controls and is easily mass manufactured by nanoimprinting. Compared with single-conduit perfusion with negative injection pressure, parallel perfusion of multiple conduits is more efficient.

Further, the drainage conduit is parallel to the extending direction of the array conduit. That is, the guiding conduit and the extending direction of the capturing conduit are consistent. The array chamber captured the particles in the liquid, and the excess liquid flowed into the drainage conduit. The drainage conduit includes a liquid storage tank on the left and right sides, configured to store the liquid after passing through the array chamber. The drainage conduit has a row of 3D re-entrant cavities. When the cross-section of cavity is semi-elliptical and inclined, the shearing force is more likely to shear the liquid, and the liquid enters the cavity. The bubbles and dead zones in the inclined cavity are less likely to be generated than in the vertical cavity. FIG. 2F shows that the oblique conduit can better reduce the pinning effect than the vertical conduit, enabling faster liquid transport. Moreover, it possesses the ability to utilize the three-dimensional surface energy gradient and Laplace pressure difference to achieve the spontaneous flow of liquid so that the liquid fills the reservoir under the capillary force of the structure of bionic Nepenthes peristome without the need for a complicated mechanical injection pump device, which has the characteristics of simple operation and high repeatability.

Upon entering the intermediate array region, the single-molecule immunocomplexes captured by the magnetic beads are subjected to the contrasting properties of the array region's surface. The hydrophilic and anti-static adsorption properties of this region, which are opposite to the hydrophobic properties of the flow separation zone, effectively prevent the non-specific adsorption of quantum dot microspheres in the array area. As a result, they continue to be expelled out of the chip with fluid dynamics. The chip, specifically designed for this purpose, efficiently removed the rest of non-specific impurities in its flow layer's separation zone. The chip's design, which leverages the hydrophobic nature of Polydimethylsiloxane (PDMS), ensures the effective removal of non-specifically bound proteins and other impurities. This design significantly improves the signal-to-noise ratio and sensitivity of the detection.

A large fraction of seeded quantum dots nanosphere adhered to the microwell surface due to a combination of surface interactions (electrostatic, van der Waals, steric, hydrophobic, and hydration forces) and therefore showed no Brownian motion.

### Analytical Validation of the Assay

In the first step of incubation, by changing different antibodies on microbeads, different target proteins were detected one by one.

The COVID-19 N-protein was selected to test the lower limit of detection and compare the performance of the digital ICA system relative to other methods. Two ocular biomarkers, TNF-α and LCN-1 were also select, to evaluate the ability of digital ICA for ophthalmology application.

To generate a standard curve for COVID-19 N-protein, TNF-alpha, and LCN-1 detection, the biomarker samples were diluted for preparation with a series of gradients and labeled the quantum dots onto the detection antibody. Then magnetic beads are combined with antigens and Quantum dots labels to form a sandwich structure complex. In this step, each of the three different types of capture microbead was placed in EP tubes on a magnetic stand to remove excess liquid, and the detection antibody solution was added to the tubes. The microbead suspensions with different standard concentrations and samples were mixed in separate tubes by shaking and incubated at room temperature for 30 min. In the wash step, Magnetic beads were removed and washed three times with PBS and Tween 20. Finally, the three magnetic beads were dispersed in 40 μL of PBS for detection.

Finally, custom-developed intelligent software was used to analyze the single molecular signal automatically. The algorithm of this software is that the Horizontal coordinate corresponds to the left-to-right and top-to-bottom position per pixel on the images. The ordinate represents the gray scatter. When the signal strength exceeds a threshold, the signal will be counted. Using 9 pictures can scan the entire array area of 200,000 particles in total and analyze more than 90% of them. Each concentration is repeated for six times before the ratio between the single molecular number and the total number of the particles is calculated to provide the Average Molecule per Bead (AMB).

The lowest detection limit in the gradient concentration data that meets linearity above 90 percent with Average Molecule Per Bead is determined.

The result shows that by using the Avidin coating system, the detection limit of COVID-19N protein reached 15 attomolar. And the limit of the detection was increased by 6-fold compared to the traditional carboxyl coating system. In different proteins, the detection limit can be affected by dissociation constant and molecular mass of different proteins. Generally, lower dissociation constant values result in higher sensitivity. Meanwhile, the sensitivity of the biomarker detection was directly related to the affinity of the antibody and aptamer.

To determine the lowest limit of detection (LOD) in a concentration gradient test, a method was employed that capitalizes on the specificity of antibody-coupled magnetic beads and quantum dots. For each concentration gradient tested, 2 μL of the magnetic bead solution was pipetted from a stock solution with a final bead concentration of 10 mg antibody-coupled beads/mL. This aliquot, containing approximately 340,000 beads, was first mixed with 10 μL of antigen sample and then diluted in pH 7.4, with 10 mM PBS to reach specific concentrations ranging from 0 fg/mL to 100 μg/mL. Samples with different concentrations were then incubated for 30 minutes at room temperature to ensure optimal binding between the antigen and the capture antibody on the magnetic beads.

Following this, the samples underwent a washing step with 150 μL of washing buffer (pH 7.4, 10 mM PBS containing 0.01% Tween-20 and 0.01% BSA) to remove any unbound antigens. The magnetic beads were subsequently isolated from the supernatant using a magnet.

Next, 2 μL quantum dot-labeled detection antibody (concentration: 25 ug/100 uL) was diluted in 100 μL of buffer solution (50 mM Tris, 0.1% BSA, and 0.05% Tween-20, pH 7.4) and then added to each sample. After ensuring thorough mixing, the samples were incubated for 30 minutes at room temperature. This incubation period facilitated the binding of the quantum dot-labeled detection antibodies to the antigens previously captured on the magnetic beads.

After incubation, another washing step was performed following the same protocol above mentioned to remove unbound quantum dot-antibody complexes. The magnetic beads were then separated from the supernatant using a magnet and resuspended in 50 μL buffer. The resuspended bead-protein-quantum dot complexes were then carefully introduced into a TF-JA (Filter-like Flow and Trap Junction array) chip using a micropipette.

All experiments were performed more than 3 times. Either duplicate or triplicate measurements were performed for the dry eyes samples at a single time point of the longitudinal cytokine profile monitoring test.

The described BiZi-FIA device was used to successfully detect coronavirus antigen N protein; as well as TNF-alpha and LCN-1.

### Example 2: Confirmation of Design Parameters for Flow-Trap Junctions

To ensure effective microbead capture by the bilateral zigzag flow, the resistance in the trap direction (Rt) should be smaller than in the flow direction (Rf). By calculating and distributing these resistance ratios, the design of flow and trap channels can be guided to optimize capture efficiency and flow performance. The resistance in the flow direction is defined as R_{f}(x, y) and in the trap direction as Rₜ(x, y), as illustrated in FIG. 2B. As the boundary conditions of FT-JA coordinate system, the origin (0,0) represents the final FT junction closest to the outlet, with the minimum resistances denoted as R_{f}(0,0)=ΔR_{fo} and Rₜ(0,0)=ΔRₜₒ, which values are influenced by the geometry of flow and trap unit respectively [23, 24]. Considering the parallel relationship between flow and trap direction at each junction, the resistance at one junction point, Rⱼ(x, y), is articulated as:

$${R}_{j} ( x , y ) = \frac{{R}_{t} ( x , y ) * {R}_{f} ( x , y )}{{R}_{t} ( x , y ) + {R}_{f} ( x , y )} . \qquad [ 1 ]$$

For positions where x≥1 and y≥0, the resistance in the flow direction, R_{f}(x, y), is determined by the resistance at the preceding junction along x-axis (flow direction), Rⱼ (x−1, y), and the resistance of the current flow unit, ΔR_{f}(x,y), which is determined by the geometry of a flow unit at (x,y):

$${R}_{f} ( x , y ) = Δ {R}_{f} ( x , y ) + \frac{{R}_{t} ( x - 1 , y ) * {R}_{f} ( x - 1 , y )}{{R}_{t} ( x - 1 , y ) + {R}_{f} ( x - 1 , y )} . \qquad [ 2 ]$$

Similarly, for x≥0 and y≥1, the resistance in the trap direction, Rₜ (x, y), is determined by the preceding junction along the y-axis (trap direction), Rⱼ (x, y−1), and the resistance of current trap unit, ΔRₜ(x,y), which is determined by the geometry of a trap unit at (x,y):

$${R}_{t} ( x , y ) = Δ {R}_{t} ( x , y ) + \frac{{R}_{t} ( x , y - 1 ) * {R}_{f} ( x , y - 1 )}{{R}_{t} ( x , y - 1 ) + {R}_{f} ( x , y - 1 )} . \qquad [ 3 ]$$

Based on these recursive relationships, the capture efficiency of the FT-JA in the BiZi-FIA system can be predicted by the resistance ratio between the trap and flow direction, Rₜ (x, y)/R_{f} (x, y). When Rₜ (x, y)/R_{f} (x, y)<1, the corresponding junction works effectively to capture the microbeads. The total resistance, Rₜₒₜₐₗ, of FT-JA is calculated via equivalent circuit simulation (FIGS. 2A-2I).

$${R}_{total} = {( {\sum }_{i} \frac{1}{{R}_{j} ( {x}_{max} , {y}_{i} )} )}^{- 1} \qquad [ 4 ]$$

Simulation results (Table 1) indicate that maintaining a higher number of trap channels relative to flow channels reduces, Rₜₒₜₐₗ, favoring microbead capture by minimizing resistance in the trap direction. Specifically, scenarios where ΔRₜ(x, y)<ΔR_{f}(x, y), achieve maximum capture efficiency. To facilitate this, we designed inclined trap channels that guide flow lines preferentially into the trap layer. In contrast, vertical trap channels increase resistance and hinder capture efficiency. Consequently, the inclined trap channel design optimizes capture efficiency within the BiZi-FIA system (FIG. 2F).

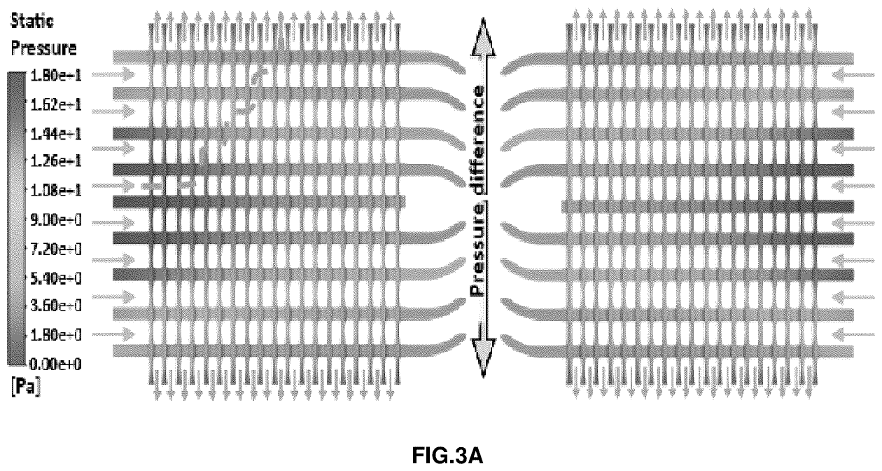

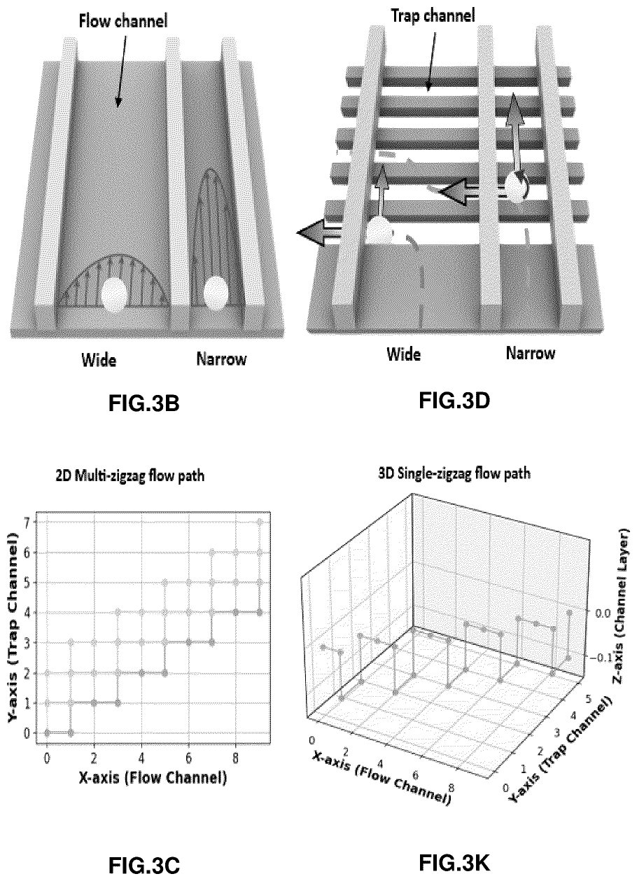

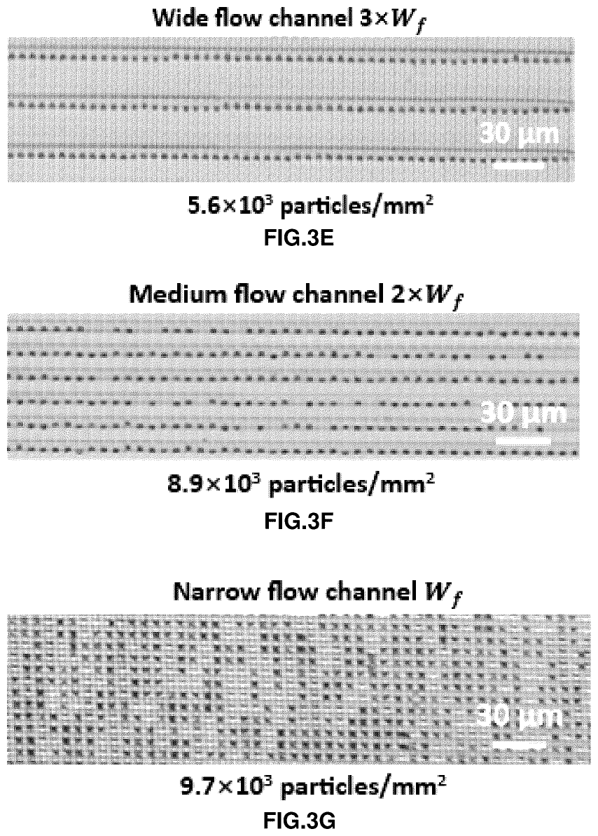

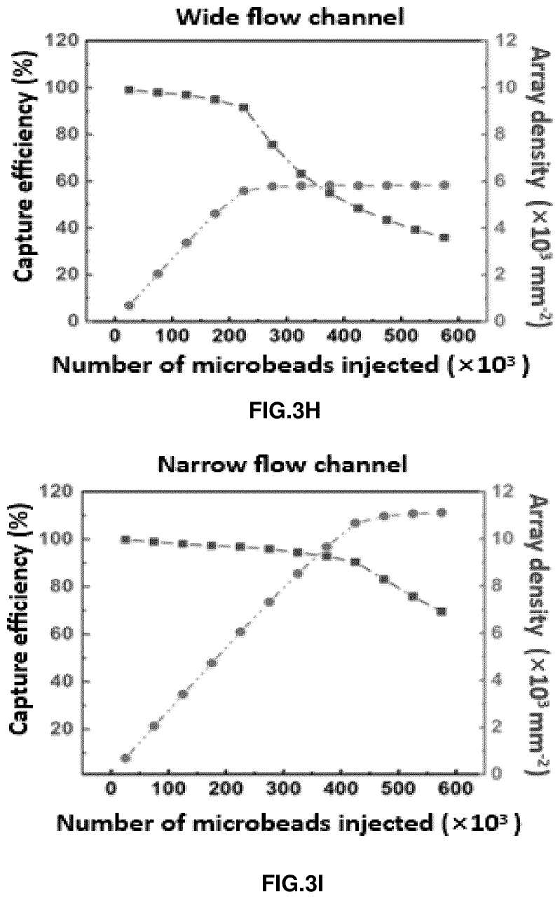

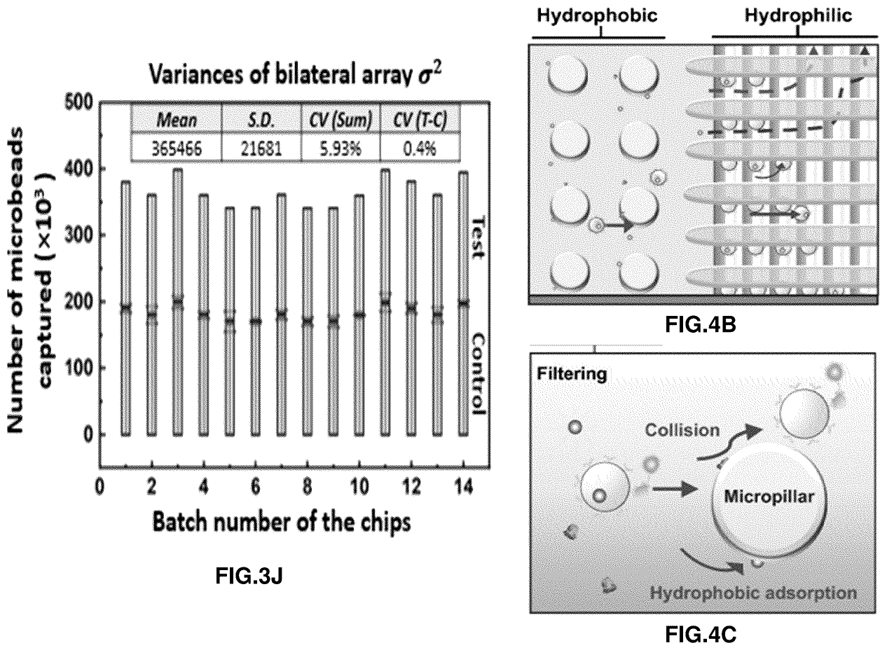

To further optimize the FT-JA system, we investigated the effect of varying the width of the flow channels. The objective was to determine whether flow channel width influences fluid velocity and, consequently, capture efficiency. The results demonstrated that while varying flow channel widths affect fluid velocity under a specific pressure difference (FIGS. 3A-3D), the overall capture efficiency remained stable (FIG. 3E-F). Specifically, at lower fluid velocities, wider channels maintained a consistent capture efficiency of 90% (n=3), allowing microbeads to be captured in an orderly and evenly spaced manner, resulting in arrays with a density of 5.6×10\hat{ }3 beads/mm² (FIG. 3E). At higher fluid velocities, narrower channels also maintained a high capture efficiency of 90% (n=3), leading to denser supplementary arrays with a density of 9.7×10\hat{ }3 beads/mm² (FIG. 3G). This stability is attributed to the optimized resistance ratios, Rₜ (x, y)/R_{f} (x, y), which ensure efficient microbead capture regardless of channel width and fluid velocity.

The FT-JA design ensures robust and efficient microbead capture, whether beads are height-restricted (Ht<d, FIG. 2G) or width-restricted (Wt<d, FIG. 2C). Experimental results (FIG. 3J) showed that maintaining a 90% capture efficiency resulted in an average of 365,466 microbeads captured across multiple batches, with a coefficient of variation (CV) of 5.9%. Within individual bilateral arrays of a single chip, the mean CV for microbead capture was as low as 0.4%, highlighting the high consistency and reliability of the BiZi-FIA system.

The FT-JA array in the BiZi-FIA system leverages hydrodynamic forces to achieve high-efficiency microbead capture and immobilization. Its filter-like flow and trap junction design ensure unidirectional flow, minimizing backflow and enhancing trapping efficiency. The inclined trap channels and parallel multi-conduit design support the formation of dense microbead arrays, maintaining high capture efficiency across varying channel widths and fluid velocities. This robust and reliable system is ideal for high-throughput and ultra-sensitive bioanalytical applications, offering a portable and cost-effective solution for single-molecule detection. Performance evaluation of the BiZi-FIA system with varying width of channel for denser microbead trapping is illustrated in FIGS. 3A-3L, showing simulated pressure gradient driving bilateral zigzag flow across FT-JA, simulated 2D multi-zigzag and 3D single-zigzag flow paths, cross-sectional view of flow channels with differing widths, and variance analysis of microbead capture in bilateral arrays, inBiZi-FIAting high stability across batches, respectively.

### Example 3: On-Chip Filtering and Washing for Magnetic-Free Immunoassay

A pivotal feature of the BiZi-FIA platform is its automated filtering and washing mechanism, which efficiently removes non-specific impurities while preserving specific immunocomplexes on the beads. The BiZi-FIA system achieves large-scale microbead arraying within an intersecting grid of double-layer Flow and Trap Junction Arrays (FT-JA). The trap channels overlap with flow channels, forming grid-like array chambers. This filter-like bead separator system effectively transports, immobilizes, and arranges microbeads into desired arrays for bioanalytical applications. It facilitates the loading and isolation of microbeads associated with single enzyme molecules using only hydrodynamic forces. Compared to single-conduit perfusion with negative injection pressure, our parallel perfusion of multiple channels significantly enhances efficiency.

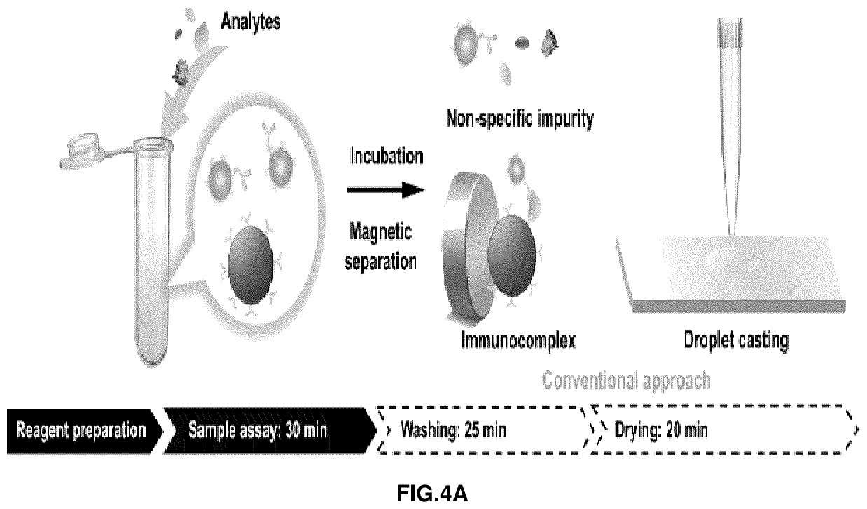

Upon entering the intermediate array region, single molecule immunocomplexes captured by the beads interact with the contrasting surface properties of the array region. The hydrophilic and anti-static surfaces prevent non-specific adsorption of quantum dot (QD) microspheres, ensuring their removal from the chip through fluid dynamics. Additionally, the hydrophobic nature of Polydimethylsiloxane (PDMS) in the flow separation zone effectively eliminates non-specifically bound proteins and impurities, significantly improving the signal-to-noise ratio (SNR) and detection sensitivity. FIGS. 4A-4C illustrates this process involves on-chip separation, where micropillars filter out non-specifically adsorbed particles. The filtering module leverages hydrophilic and hydrophobic surfaces to enhance collision-based filtering, significantly reducing non-specific adsorption. The enhanced specificity of the platform is evident in the fluorescence detection phase, where beads arrayed in the detection chamber show minimal non-specific signals, even in negative control samples (0 fg/mL). Conventional methods relying on magnetic setup suffer from higher noise levels due to incomplete removal of non-specifically nanosphere. By automating the filtration process and improving bead arraying, the BiZi-FIA platform ensures high throughput and efficient detection of even ultra-low concentrations of target molecules. A exemplary process of the BiZi-FIA platform for single-molecule detection with automatic filtering and washing mechanism is depicted in FIG. 4, showing reagent preparation and incubation in the BiZi-FIA workflow, conventional separation method with magnetic device, droplet casting for immunocomplex analysis, BiZi-FIA schematic showing filtering and washing mechanisms, using hydrophobic and hydrophilic surfaces for efficient filtering, and a fluorescence image of BiZi-FIA, showing effective removal of non-specific impurities and clear separation of signals in negative controls (0 fg/mL). Conventional magnetic washing retains non-specific nanospheres, increasing background noise, and fluorescence detection of target molecules (10, 20, and 40 fg/mL), demonstrates distinct signal spots and uniform microbead alignment in magnified views.

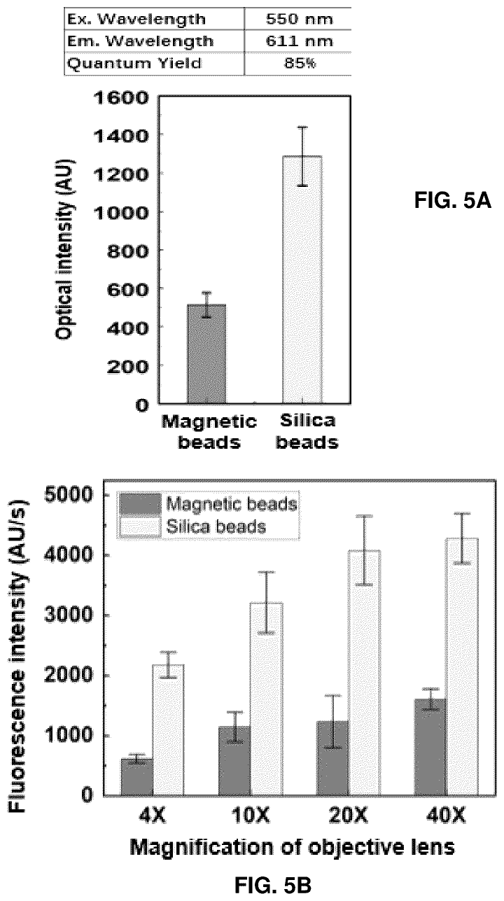

The BiZi-FIA platform, employing silica beads as the primary capture substrate, demonstrates a marked improvement in signal-to-noise ratio (SNR) compared to traditional magnetic bead-based assays. This enhancement is driven by the higher optical clarity of silica beads and reduced tendency for non-specific binding, which is a key factor in lowering background noise. As shown in FIGS. 5A-5B, silica beads were selected for their enhanced light transmissivity, which significantly boosts the detection sensitivity of QD-labeled antibodies. This is demonstrated in the comparison of fluorescence microscopy images, where silica beads exhibit a higher density of visible QD-labeled events than magnetic beads. This improvement is attributed to the minimized light attenuation and quenching typically associated with magnetic beads. Optical intensity measurements by fluorescence spectrophotometer at 611 nm (FIG. 5A) further confirm the advantage of silica beads, with fluorescence intensities almost 2-3 times those observed with magnetic beads. This enhancement is particularly beneficial for single-molecule detection, where the ability to differentiate between true signals and background noise is critical. Microscopic imaging also highlights the superior signal uniformity across individual silica beads, underscoring the ability to maintain high SNR across different magnification levels (FIG. 5B).

### Example 4: Superior Signal-to-Noise Ratio Based on Silica Beads for Large Area Rapid Imaging by CMOS Sensor

To quantitatively understand the superior performance of silica beads and compare the minimum requirements for the optical system that enables the detection of single-molecule fluorescence signals on different bead types, we analyzed various parameters affecting signal detection capability. These parameters include the numerical aperture (NA) of the objective lens, exposure time, and detector sensitivity, particularly focusing on the detection limits of the CMOS sensor used in commercial fluorescence microscopy. The photon emission rate (Rₑₘ) of quantum dots (QDs) on beads is determined by the photon absorption rate (R_{abs}) and the quantum yield (QY):

$${R}_{e m} = QY \times {R}_{a b s}$$

Both silica and magnetic beads were around 3 μm in diameter and labeled with QDs exhibiting a high quantum yield (QY) of 85% with excitation and emission wavelengths at 550 nm and 611 nm respectively (from the data of manufacturer DIAGNANO™, typically measured using fluorescence spectrometry under specific excitation conditions). The photon emission rate (Rₑₘ) from a single QD is calculated using:

$${R}_{e m} = QY \times \frac{{σ}_{a b s} \times {I}_{0}}{h {v}_{e x c}}$$

### Where:

σ_{abs} is the absorption cross-section of QDs (1×10⁻¹⁶ cm², the date from the datasheet provided by manufacturer DIAGNANO™, typically measured through experimental measurements of the QDs' absorption properties at the specific wavelength of 550 nm). I₀ is the excitation light intensity (1×10⁵ W/cm²), equal to the ratio between the power of a laser beam (10 mW) and the area of the light spot;
hv_{exc} is the individual photon energy at the excitation wavelength;
h is Planck constant (6.626×10⁻³⁴ J·s); and
v_{exc} is the photon frequency (5.45×10¹⁴ Hz), which is equal to the ratio between the velocity of light and excitation wavelength.

For silica beads, which are highly transparent and exhibit minimal quenching, the calculated photon emission rate (R_{em,silica}) is approximately 2.35×10⁷ photons/s. In contrast, magnetic beads introduce significant light absorption, scattering, and quenching due to their magnetic materials (iron oxide). These effects reduce the effective quantum yield and the excitation/emission efficiencies by approximately 50%.

$$e {R}_{e m , m a g n a t i c} = {R}_{a b s} \times QY \times QF \times {A}_{e x c}$$

### Where:

QF is a quenching factor, represents a reduction in quantum yield due to energy transfer processes or other quenching mechanisms caused by the proximity of the magnetic material; and
A_{exc} is attenuation of excitation light due to absorption/scattering by the magnetic bead.

Consequently, photon emission rate of magnetic beads (R_{em,magnatic}) is reduced to about 5.89'10⁶ photons/s, which is used to determine the number of photons detected (N_{detected}):

$${N}_{d e t e c t e d} = {R}_{e m} \times {A}_{e m} \times T \times {η}_{d e t e c t o r} \times τ \times {f}_{c o l lection}$$

### Where:

Aₑₘ is the emission attenuation factor, which was measured experimentally. Represents the reduction in emitted fluorescence intensity due to absorption and scattering of emitted photons by the bead material before they reach the detector;
T is the overall transmission efficiency of the optical system, accounting for losses due to optical components (50% or 0.5, based on typical losses in the optical components used e.g., lenses, filters);
η_{detector} is the quantum efficiency of the detector (CMOS sensor) at the emission wavelength of 611 nm (50% or 0.5, based on the specifications of detector provided by the microscope manufacturer);
τ is the exposure time (1 second, chosen based on the requirements of the experimental design and the need to balance signal acquisition with imaging speed); and
f_{collection} is the fraction of emitted photons collected by the objective lens, depending on its numerical aperture NA of the lens:

$${f}_{collection} = \frac{1 - \sqrt{1 - N {A}^{2}}}{2} .$$

To meet the application requirement for precise quantification of single-molecule signal, we set a high SNR threshold of 100 that provides a high level of confidence in distinguishing the signal from noise. The minimum number of detected photons (N_{detected}) required to achieve the signal-to-noise ratio (SNR) of 100 is approximately 10,025 photons. This is determined using the SNR definition formula:

$$SNR = \frac{{N}_{d e t e c t e d}}{\sqrt{{N}_{d e t e c t e d + {σ}_{r e a d}^{2}}}}$$

Where: σ_{read} is the read noise of the detector in electrons rms. (σ_{read}=5 rms, obtained from the technical specifications of microscope CMOS detector).

To achieve the required N_{detected}, the minimum fraction of collected photons (f_{collection}) must satisfy:

$${f}_{c o l lection} \geq \frac{{N}_{d e t e c t e d}}{{R}_{e m} \times T \times {η}_{d e t e ctor} \times τ}$$

The minimum NA is then calculated using:

$$N A \geq \sqrt{2 \times {f}_{c o l lection}}$$

Based on this relationship, Table 2 lists different objective magnifications, their typical numerical apertures (NAs), and the corresponding calculated result of signal-to-noise ratios (SNRs) for both silica beads and magnetic beads. This table helps visualize how the SNR varies with different optical system configurations for each bead type.

TABLE 2
Quantification of SNR between silica beads and magnetic
beads among different magnification of objective lens

| Magnification | NA | f_{collection} | N_{detected silica} | SNR_{silica} | N_{detected mag} [symbol] | SNR_{magn} [symbol] |
|---|---|---|---|---|---|---|
| 4× | 0.10 | 0.0025 | 14,687.5 | 121.0 | 1,836.0 | 42.56 |
| 10× | 0.25 | 0.0311 | 182,722.5 | 427.3 | 22,840.3 | 151.1 |
| 20× | 0.40 | 0.0804 | 472,940.0 | 687.3 | 59,117.5 | 243.1 |
| 40× | 0.65 | 0.2113 | 1,243,856.3 | 1,115.1 | 155,482.0 | 394.4 |
| 60× | 0.85 | 0.3201 | 1,885,660.6 | 1,373.7 | 235,707.6 | 485.5 |
| 100× | 1.30 | 0.2436 | 1,429,950.0 | 1,195.6 | 178,743.8 | 423.0 |

*The magnification of the eyepiece (usually 10×) has no direct effect on these values. [symbol]
indicates data missing or illegible when filed

For silica beads, calculations show that an NA as low as 0.1 is sufficient for silica beads, which is easily achievable with standard low-magnification objectives (e.g., a 4× objective with NA≈0.1). For magnetic beads, due to the reduced emission rate, the required f_{collection} is higher, leading to a minimum NA of approximately 0.25. This indicates that detecting single-molecule signals on magnetic beads necessitates a higher NA or additional optimization, such as increased exposure time or enhanced detector sensitivity. The lower NA requirements for silica beads facilitate large field-of-view imaging, as low-magnification objectives with wide fields can be used without compromising detection sensitivity. This advantage is crucial for applications requiring the analysis of large populations of beads or widespread distribution of targets. The BiZi-FIA platform, employing silica beads as the primary capture substrate, demonstrates a marked improvement in signal-to-noise ratio (SNR) compared to traditional magnetic bead-based assays. This enhancement is driven by the higher optical clarity of silica beads and reduced tendency for non-specific binding, which is a key factor in lowering background noise. As shown in FIGS. 5A-5B, silica beads were selected for their enhanced light transmissivity, which significantly boosts the detection sensitivity of QD-labeled antibodies. This is demonstrated in the comparison of fluorescence microscopy images, where silica beads exhibit a higher density of visible QD-labeled events than magnetic beads. This improvement is attributed to the minimized light attenuation and quenching typically associated with magnetic beads. Optical intensity measurements by fluorescence spectrophotometer at 611 nm (FIG. 5A) further confirm the advantage of silica beads, with fluorescence intensities almost 2-3 times those observed with magnetic beads. This enhancement is particularly beneficial for single-molecule detection, where the ability to differentiate between true signals and background noise is critical. Microscopic imaging also highlights the superior signal uniformity across individual silica beads, underscoring the ability to maintain high SNR across different magnification levels (FIG. 5B). The theoretical calculations corroborate the experimental observations in FIG. 5B, confirming that the superior fluorescence intensity of silica beads arises from their excellent optical properties which are high transparency and minimal quenching. These properties allow for efficient excitation and emission of fluorescence signals, enabling the detection of single-molecule events even with low-NA objectives and standard CMOS detectors. In contrast, magnetic beads require more stringent optical conditions due to their inherent attenuation and quenching effects. The enhanced detection capability provided by silica beads not only improves the signal-to-noise ratio (SNR) but also expands the flexibility of the optical system design. Their use allows for low-magnification, wide-field objectives, which are advantageous for large field-of-view imaging applications. Silica beads-enhanced fluorescence for extensive field of view detection was observed in a comparison of signal intensities between magnetic beads and silica beads under brightfield microscopy, together with fluorescence microscopy images of the same fields of view (4× objective lens) for magnetic beads and silica beads. The images showed a significantly higher density of visible QD-labeled detection events on silica beads. Optical intensity comparison at a wavelength of 611 nm between magnetic and silica beads, showed a marked increase in signal for silica beads. Microscopic imaging of individual beads at high magnification highlighted better fluorescence signal uniformity and intensity on silica beads compared to magnetic beads, and fluorescence intensity comparison across various magnifications (4×, 10×, 20×, 40×) showed the superior performance of silica beads in enhancing signal detection.

### Example 5: Silica Bead Usage, Analyzed Fraction and SA-Biotin Reaction System for Optimizing Sensitivity

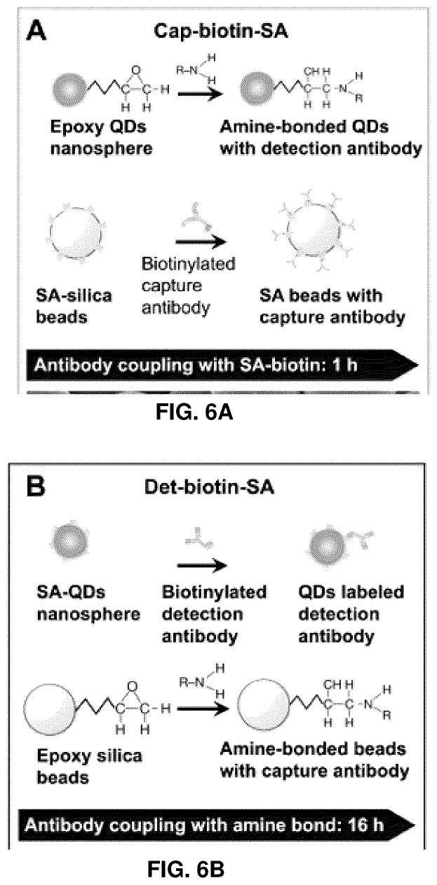

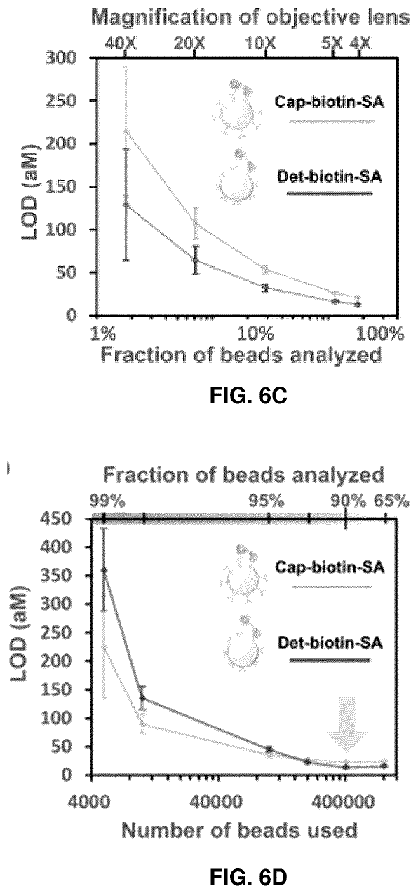

Beyond bead usage, the application of the streptavidin (SA)-biotin system to either the capture (Cap-biotin-SA, FIG. 6A) or detection (Det-biotin-SA, FIG. 6B) components further influences assay performance. When the SA-biotin system is applied to capture sites (Cap-biotin-SA, FIG. 6A), detection antibodies are functionalized via amine bonds on QDs nanosphere labels. Conversely, when the SA-biotin system is employed on detection sites (Det-biotin-SA, FIG. 6B), capture antibodies are functionalized via amine bonds on silica beads. Scanning Electron Microscopy (SEM) images in FIG. 6A-6B demonstrate consistently functionalized surfaces, ensuring a homogeneous distribution of capture sites across all beads. Specifically, when the SA-biotin system is utilized on capture beads, measurements yielded a mean diameter of 3.03 μm with a range of 2.97-3.08 μm. In contrast, when amine bonds are used on capture beads, measurements yielded a mean diameter of 3 μm with a range of 2.94-3.04 μm. When relatively fewer capture microbeads are used, the use of SA-biotin to the capture beads increases the number of available binding sites and reduces steric hindrance, improving detection sensitivity and lowering the limit of detection (LOD) (FIG. 6D). However, as the number of capture beads increases and the binding sites approach saturation, the further addition of the use of beads yields diminishing returns. Sensitivity can decrease when bead usage exceeds the loading capacity, as some beads cannot be analyzed (FIG. 6D).

When the number of microbeads exceeds 200,000, resulting in saturation of the capture sites, transferring the SA-biotin system to the detection sites can enhance the sensitivity by 30% compared to the SA-biotin-Cap system (FIG. 6C). This improvement in sensitivity and lower LOD values are primarily due to the optimized binding interactions and more efficient signal generation from the detection antibody. The Det-biotin-SA system ensures that each immunocomplex is effectively tagged with a single, highly fluorescent nanosphere, thereby maximizing the signal output per binding event and improving overall assay sensitivity.

At a high fraction of beads being analyzed (e.g., 90%), the Det-biotin-SA system achieves a limit of detection (LOD) below 13 aM for IgE detection using 400,000 beads (FIG. 6C). This performance surpasses that of the Cap-biotin-SA system, which reaches a LOD of approximately 22.5 aM with the same number of beads. Although Cap-biotin-SA provides advantages at lower bead usage by alleviating spatial constraints on capture sites, its benefits in enhancing detection sensitivity diminish once bead saturation is reached. In contrast, Det-biotin-SA becomes more effective as bead density increases, demonstrating superior signal generation under high-density conditions and outperforming Cap-biotin-SA (FIG. 6D). These effects are fundamentally driven by a balance between the availability of capture versus detection sites, as well as the interplay between their intrinsic association and dissociation kinetics. Increasing the number of capture sites enhances sensitivity up to a certain point. This improvement continues only until the association and dissociation kinetics of detection sites collectively reach a saturated state. In other words, when the reaction rate of the detection site cannot be further improved, increasing the capture site will no longer bring about an increase in sensitivity. Beyond this saturation point, adding extra beads no longer enhances and may even detract from overall analytical efficiency.

The results demonstrated that objective magnification significantly impacts the limit of detection (LOD) and standard deviation (SD) in the BiZi-FIA platform. Utilizing a high bead concentration of 400,000 beads, which ensures minimal gaps and maintains a capture efficiency exceeding 90% were evaluated for the effect of objective magnification on detection sensitivity after a 30-minute incubation period.

As the fraction of beads analyzed increases from 1% to 90% by decreasing the objective magnification from 40× to 4×, the LOD decreases substantially, reaching its lowest value when approximately 90% of the beads are analyzed. In contrast, higher objective magnifications (e.g., 40× and 20×) result in significantly higher LODs. This increase is attributed to the smaller field of view at higher magnifications, which limits the number of beads analyzed and reduces the statistical robustness of the detection.

Additionally, the coefficient of variation (CV) increases substantially at higher objective magnifications, reaching up to 70% at 40× magnification of the objective lens. This indicates greater variability and reduced reliability of the measurements using a high-magnification objective lens. Similarly, the SD of the detection measurements increases, which reflects the greater dispersion due to the limited sample size. Objective lenses with lower magnifications allow for the analysis of a larger number of beads within a single field of view, therefore significantly reducing the LOD, CV, and SD. By increasing the fraction of beads analyzed, detection sensitivity is enhanced as measurement uncertainty is reduced. The data indicates that analyzing approximately 60% of the beads can achieve reliable detection with a CV of less than 5%. This suggests that scanning a single image with a 4× or 5× objective lens magnification across the assay area is sufficient for reliable quantification using the BiZi-FIA system (FIG. 6C)

Strategic application of the SA-biotin system was carried out for analysis of BiZi-FIA platform sensitivity, and array density under varying conditions was assessed. A schematic of the capture antibody labelling method with SA-biotin system is depicted in FIG. 6A, and SEM images illustrating SA-biotin-functionalized silica beads is depicted in FIG. 6A. A schematic of reverse setup for detection antibodies labelling method with SA-biotin system is depicted in FIG. 6B, and SEM images illustrating amino-functionalized silica beads is depicted in FIG. 6B. Under the condition of 400,000 beads used, the variation of LOD with different fractions of beads analyzed, showing optimal performance with higher fractions and lower objective lens magnification. (FIG. 6D). The relationship between limit of detection (LOD) in attomoles (aM) and the number of beads used to demonstrate increased sensitivity with higher bead density was assayed. With the number of beads used increased above the capturing ability, a certain number of beads would be accelerated lost, which leads to a decrease in the fraction of microbeads analyzed. Microscopic images displaying array densities at increasing bead numbers under a 20× objective lens, illustrated the uniformity and scalability of bead distribution in high-density arrays.

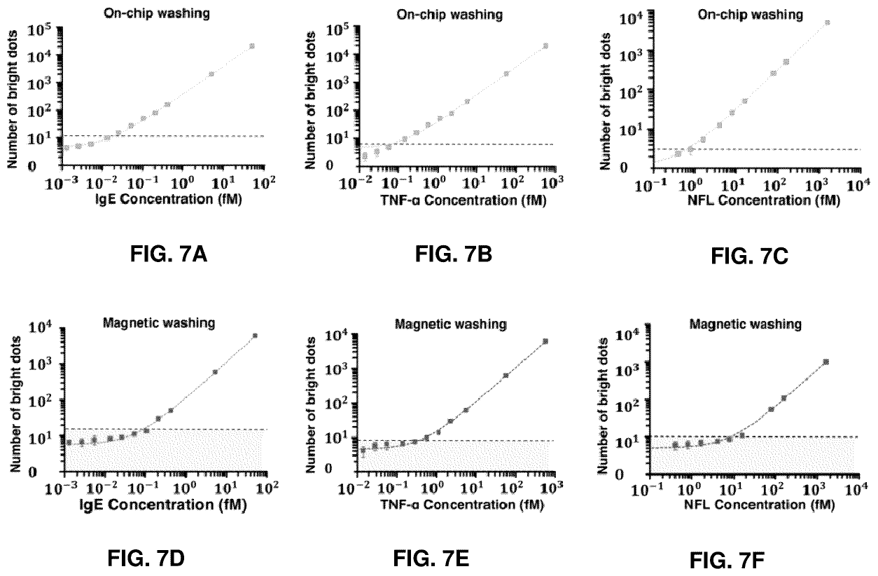

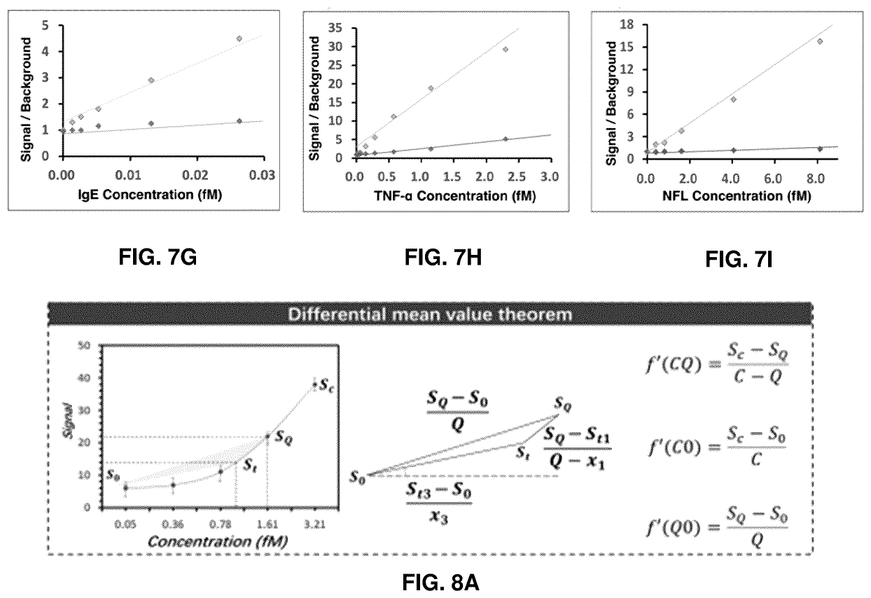

At an objective magnification of 4× with 400,000 silica beads, the optimized detection condition for the Det-biotin-SA system offers stable and ultra-sensitive detection. This optimization was essential for accurately quantifying three target proteins namely immunoglobulin E (IgE), tumor necrosis factor-alpha (TNF-α), and neurofilament light chain (NFL). These proteins are critical biomarkers for allergic responses, immune defense in the eye, and inflammatory responses, respectively. Specifically, IgE is a key immunoglobulin involved in allergic reactions, primarily mediating hypersensitivity responses by binding to allergens and triggering the release of histamine and other inflammatory mediators from mast cells and basophils, TNF-α possesses antibacterial and anti-inflammatory properties and plays a role in the immune defense of the eye, making it particularly valuable for diagnosing dry eye syndrome [28]. NFL serves as an indicator of the active degree of inflammatory response, especially the activation and migration of neutrophils. The human forms of the protein spiked into synthetic tear solutions (SYSTANE®) to final concentrations representative of clinical test samples. As LOD is determined by extrapolating the concentration at background plus 3 SD of the background, LODs for different runs are dependent on the CV of the background. After a 30-minute incubation, the optimized detection condition significantly improved both the limit of detection (LOD) and the reliability of measurements. Specifically, the LOD for IgE was reduced from 0.21 fM (40 fg/mL) with magnetic beads (FIGS. 7D-7F) to 0.013 fM (2.5 fg/mL) using silica beads (FIG. 7A-7C). Similarly, for TNF-α, the LOD using silica beads (FIG. 7B) decreased to 0.05 fM, significantly lower than the 0.57 fM achievable with magnetic beads in conventional digital immunoassays (FIG. 7E). For NFL, the sensitivity improvement lowered the LOD from 16.27 fM (FIG. 7F) to 0.81 fM (FIG. 7C).

In summary, the BiZi-FIA system overcomes the limitation of magnetic beads-based immunoassay by introducing a streamlined process for arranging silica beads into dense, uniform arrays that maximize detection efficiency. The silica bead-based BiZi-FIA platform offers a robust and highly sensitive solution for single-molecule detection, with superior optical performance and a dramatically enhanced signal-to-noise ratio compared to traditional magnetic bead-based immunoassay systems. This advancement holds a particular promise for clinical diagnostics, where early and accurate detection of biomarkers is critical for improving patient outcomes.

Comparisons of sensitivities of silica beads based on BiZi-FIA chip washing and magnetic beads based on conventional magnetic washing were determined. BiZi-FIA and conventional Digital immunoassay calibration curves for IgE, TNF-α, NFL were calculated, as depicted in FIGS. 7A-7F. Dashed lines indicates the calculated limits of detection (LODs). Comparison of signal to background ratios between silica beads and conventional magnetic beads across the calibration curve range for both the BiZi-FIA and digital Immunoassays are depicted in FIGS. 7G-7I.

### Example 6: Computational Methods for Adaptive Differential Noise Correction with BiZi-FIA for Broad Dynamic Range Rapid Precision Detection

The Adaptive Differential Noise Correction (ADNC) algorithm was developed to enhance the precision and reliability of the Bilateral Zigzag Flow Immunoassay (BiZi-FIA) system in detecting ultra-low concentration proteins. The ADNC algorithm leverages the symmetrical bilateral assay setup of BiZi-FIA, where test and control samples are introduced from opposite sides of the flow channel, ensuring similar environmental exposure for both samples. This symmetry is pivotal in accurately distinguishing true signal from background noise. The implementation of ADNC involved several key steps, including data acquisition, differential analysis, noise modeling, and iterative optimization using gradient descent. The mathematical framework for the ADNC algorithm is grounded in the Poisson distribution, which is suitable for describing sparse events like single-molecule protein detection signals. Fluorescent signals from the test sample (Sₜ) and the control sample (S_{c}) are modeled as independent Poisson-distributed variables. Background noise (S₀), a component of both the test and control signals, is also assumed as Poisson-distributed. Importantly, the control sample (S_{c}) contains background noise but may also include signal contributions from known concentrations of a standard.

To refine detection accuracy, the algorithm calculates the gradient of the signal difference between test and control samples using:

$${f}^{′} ( TC ) = ( {S}_{t} - {S}_{C} ) / ( x - C )$$

where x and C are the concentrations of the test and control samples, respectively. This differential analysis accounts for nonspecific adsorption and isolates the true signal. Systematic experiments are conducted across various known concentrations to train the ADNC algorithm through differential analysis. Data from bilateral assays is collected to establish relationships between test and control areas. Test samples highlight experimental variations at specific concentrations, forming the basis for gradient analysis, which normally caused by “pH”: pH variations, “T”: temperature fluctuations, “k_{off}”: dissociation constant of specific antibody, “γ”: interfacial energy, and “I”: ionic concentration changes. “Nᵤₛₑ”: number of microbeads used, “Nₐₙₗ”: number of microbeads analyzed, as shown FIGS. 8A-8C.

During the training process, the algorithm learns to adjust test readings based on control responses. The software has an initial setup, which involves inputting the concentration values of quantitation sample (Q) and control samples (C). The process begins with a bilateral assay of the test sample against the quantitation sample to obtain the first test signal (Sₜ₁) and the quantitation signal (S_{Q}). This is followed by a bilateral assay with a control sample (C) to get the second test signal (Sₜ₂) and the control signal (S_{C}). Finally, a bilateral assay with a blank sample provides the third test signal (Sₜ₃) and the background signal (S₀). After repeating the bilateral assays, the software records the signal gradients f′(CQ), f′(C0), and f′(Q0) for the specific biomarker as follows:

$${f}^{′} ( CQ ) = \frac{{S}_{c} - {S}_{Q}}{C - Q}$$

$${f}^{′} ( C 0 ) = \frac{{S}_{c} - {S}_{0}}{C}$$

$${f}^{′} ( Q 0 ) = \frac{{S}_{Q} - {S}_{0}}{Q}$$

During the initial setup, software will iterate through the threshold range to ensure that

$${f}^{′} ( C Q ) \geq {f}^{′} ( C 0 ) \geq {f}^{′} ( Q 0 )$$

which can be understood in the context of Lagrange's differential meaning value theorem [30], explaining the underlying connection between the average rate of change in the function and its instantaneous rates at different points (FIG. 8).

By calculating the mean difference in signal intensity between consecutive points in the assay field, the algorithm can fine-tune its correction factor, δ, to address local variations caused by measure error. Based on the available concentrations (C, Q) and the corresponding fluorescence detection signals, we establish functions for x in terms of δ.

$${x}_{1} = Q + \frac{{S}_{t 1} - {S}_{Q}}{{δ}_{1} {f}^{′} ( C Q )}$$

$${x}_{2} = C + \frac{{S}_{t 2} - {S}_{c}}{{δ}_{2} {f}^{′} ( C 0 )}$$

$${x}_{3} = \frac{{S}_{t 3} - {S}_{0}}{{δ}_{3} d {f}^{′} ( Q 0 )}$$

Next, validation is performed using cross-validation techniques to assess the accuracy of the model. Parameters are fine-tuned through gradient descent optimization, minimizing variance among concentration estimates across repeated tests. The loss function, L(x, x̅), used in the optimization process is defined as:

$$L ( x , \overline{x} ) = {\sum }_{i = 1}^{n} {( {x}_{i} - \overline{x} )}^{2}$$

where xᵢ represents individual concentration estimates, and x̅ is their mean value. The correction factor δ is iteratively updated using:

$${δ}_{n e w} = {δ}_{o l d} - α \frac{\partial L}{\partial δ}$$

This iterative process ensures convergence to an optimal correction factor that minimizes discrepancies and gradually reduces the loss, helping it find the optimal model parameters δ₁, δ₂, δ₃. This process involves calculating the partial derivatives of the loss function L(x, x̅) concerning δ and iteratively updating the values of δ based on these derivatives, until the L(x, x̅)≤ε. δ_{old} refers to the current value of the parameter from the previous iteration. ∝L/∝δ is the gradient of the loss function with respect to the parameter, indicating how the loss function changes with small changes in δ. α is the learning rate that determines the size of each step taken during the update. δ_{new} is the updated parameter value used in the next iteration. Typically, “1” is used as the initial value of 8, starting with the ideal state. Using the optimized correction factor (δ1, δ2, δ3), the program calculates the values of the unknowns x₁, x₂, x₃. Finally, the program outputs precise concentration by taking the mean value, x (FIG. 8). After three repeated bilateral assays, a standard curve function, f(x), with the optimized signal gradient f′(x) is established for specific biomarkers, allowing followed sample tests with a bilateral assay to calculate the concentration quickly. The software automatically verifies the accuracy of the concentration output results, as shown in FIG. 8 D. If it detects values exceeding a reasonable concentration range, the system will output “invalid data” instead of generating erroneous data. By following these steps, the ADNC model assists understanding and correcting of the errors caused by environmental variables for single-molecule protein detection, thereby improving the accuracy and reliability of the detection.

The Adaptive Differential Algorithm (ADA) operating interface of the ADNC software for concentration calculations is depicted in FIGS. 8A-8C. The interface displays fluorescence image inputs for blank, quantitation, control, and test samples. Bright spot counts and signals are automatically processed to compute concentration gradients (f′(CQ), f′(C0), f′(Q0)) using the ADNC algorithm. A standard curve plots known points and fitted signals, enabling precise concentration calculations for ultra-low concentration protein detection. A differential mean value theorem for signal interpretation in adaptive corrections is depicted in FIG. 8A. The fluorescence intensity versus concentration curve illustrates relationships between S_0 (background noise), S_Q (quantitation sample signal), S_C (control sample signal), and S_t (test sample signal). Gradient differentials (f′(CQ), f′(C0), f′(Q0)) derived from concentration changes are critical for adaptive corrections. The shaded region highlights the difference between fluorescence units for accurate gradient optimization. Examples of fluorescent signal distributions across different sample types for training the ADNC are depicted in FIG. 8C. The red fluorescence dots represent the raw emission signal captured from the samples, while the green circles indicates signals that surpass the certain threshold and are subsequently identified as valid events by software. Blank sample (S₀), quantitation sample (S_Q), control sample (S_C), and test samples (S_t1, S_t2, S_t3) show varying signal distributions. A flowchart outlining the key steps of the ADNC algorithm, including input setup, signal switching, and gradient threshold validation is depicted in FIG. 8B. A 3D loss function surface plot (L(x)) demonstrates the iterative gradient descent process for minimizing discrepancies in unknown test sample concentrations (x₁, x₂, x₃) is depicted in FIG. 8B. The algorithm ensures optimal calibration for robust signal corrections and accurate concentration predictions.

### Example 7: Detection of Biomarkers: Clinical Trials for Combined Analysis of Multiple Biomarkers

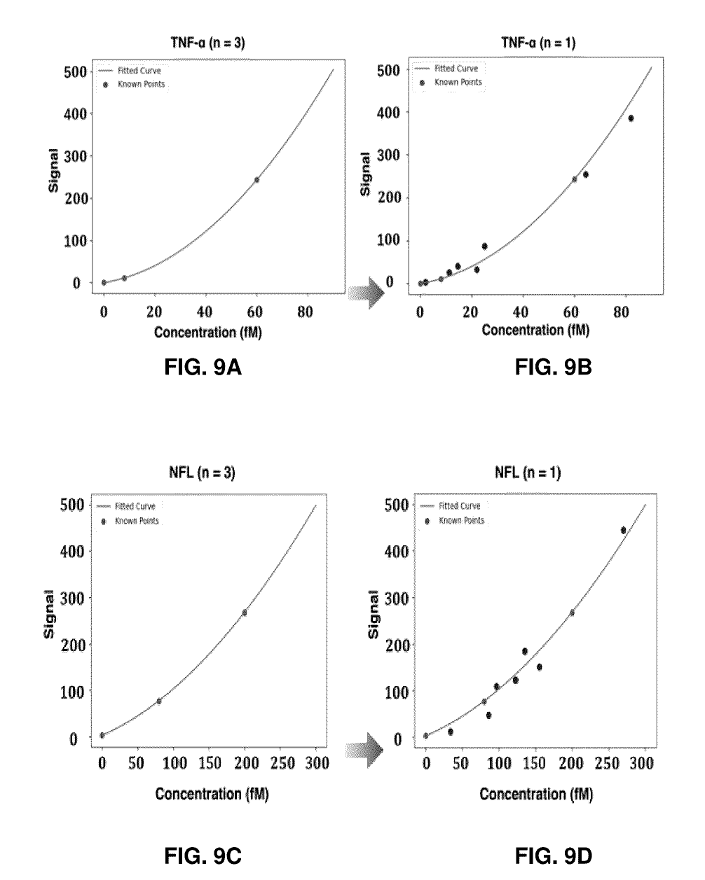

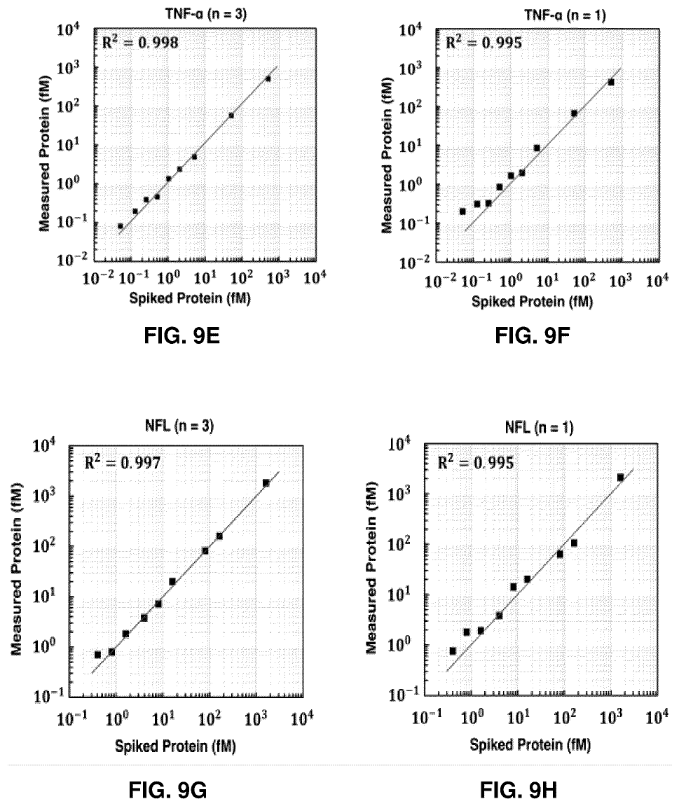

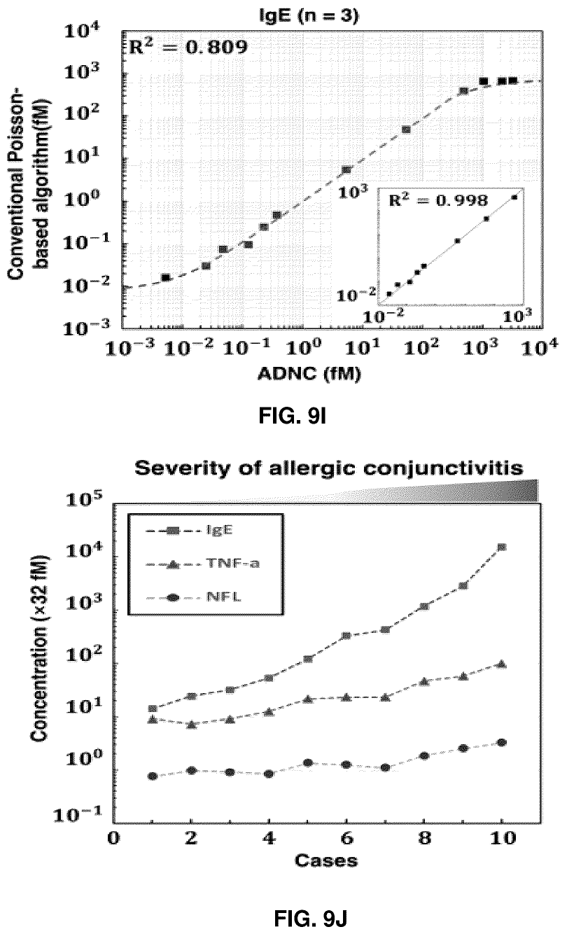

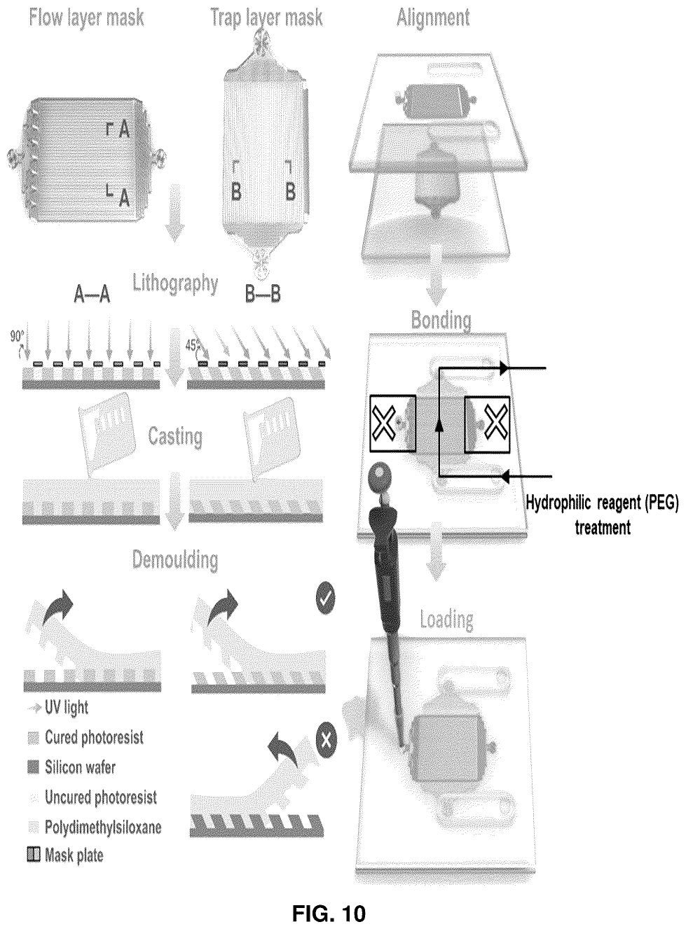

To evaluate the quantification accuracy and reproducibility of the BiZi-FIA-ADNC system, calibration curves for the biomarkers TNF-α and NFL were established using artificial tear samples spiked with varying concentrations of these proteins. As shown in FIG. 9A, 9C, the Adaptive Differential Noise Correction (ADNC) software generated standardized concentration-response curves using three control samples (data points) with triplicate measurements of a test sample. Triplicate measurements (n=3) of TNF-α and NFL produced consistently strong linearity with coefficients of determination (R²) exceeding 0.997 (FIG. 9E, 9G). While single-run measurements (n=1) of dynamically introduced test samples maintained near-perfect alignment with the calibration baselines (reference lines), achieving R² values of 0.995 for TNF-α at the range of 0.01 fM-1000 fM and NFL at the range of 0.1 fM-10000 fM (FIG. 9F, 9H). The measured concentrations showed quantitative recovery rates of 92-107% across clinically relevant ranges. This demonstrates the robustness against matrix effects in complex biological fluids. Then ADNC algorithm was further validated by comparing its performance against traditional Poisson-based signal processing methods for detecting IgE concentrations. The ADNC algorithm outperformed traditional Poisson-based signal processing in critical aspects. First, ADNC expanded the linear dynamic range for immunoglobulin E (IgE) detection to 0.01-10,000 fM, a two-log improvement compared to the 0.024-526 fM range achievable with Poisson methods (FIG. 9I). The comparison highlights the advantages of ADNC in achieving an extended range of detection, particularly at low concentrations below 1 fM and high concentrations above 600 fM, where traditional Poisson-based methods begin to lose linearity. Notably, the inset graph emphasizes the consistency of the result between the ADNC and the Poisson-based signal processing method during a certain range of 0.024-526 fM. Second, the algorithm eliminated dependency on brightfield imaging by enabling full field darkfield analysis across a 5.5×5.5 mm² FOV 2 field of view at 4× magnification, contrasting with the constrained imaging area required for conventional microbeads counting at 10× magnification. The data highlights the distinct advantages of the ADNC algorithm over the traditional Poisson-based signal processing method in terms of signal analysis workflow and efficiency. The control area reflects the extent of background noise and real signal, while the test area provides measurements of the test signal and noise. This capability of the ADNC algorithm provides sufficient data for robust noise correction and concentration calculation significantly enhances accuracy and efficiency in biomarker detection. This not only accelerates the signal imaging process but also provides sufficient data for signal analysis, thereby improving the overall robustness and reliability of the system, broadening the dynamic range of detection. By leveraging the ADNC algorithm, the BiZi-FIA system eliminates the dependency on limited-field brightfield imaging and extends its capabilities for more rapid and wider dynamic range detection. Finally, ADNC achieved a minimal deviation (<5% signal variance) between calibration and rapid-test phases, demonstrate a robust linear relationship between fluorescence signal intensity and biomarker concentration, highlighting the sensitivity and precision of the system in detecting ultra-low protein concentrations.

Additionally, multiplexed biomarker quantification was validated using 2.2 μL tear samples from two prospectively recruited cohorts: a control group of five asymptomatic individuals (Ocular Surface Disease Index [OSDI]≤12, tear film breakup time [TFBUT]>10 s) and a disease cohort comprising five patients with clinically confirmed dry eye (OSDI≥33, TFBUT≤5 s) with moderate-to-severe allergic conjunctivitis (Clement grade II-III). The system resolved biomarker concentrations spanning five orders of magnitude, with IgE detected from 14 fM to 15,388 fM, TNF-α from 53 fM to 486,945 fM, and NFL from 24 fM to 1,184 fM. This performance highlights the capability to quantify biomarkers across diverse physiological ranges within complex biological matrices.

The BiZi-FIA method was compared to LFIA (Lateral Flow Immunoassay) for IgE detection where BiZi-FIA consistently outperformed LFIA across all tested cases. The platform demonstrated a 1,000-fold sensitivity improvement over LFIA, detecting IgE at 13 aM compared to the threshold of LFIA at 1 IU/mL, as indicated by the specification of manufacturer (Seinda Biome Corporation). Furthermore, BiZi-FIA achieved 32-plex detection in under 40 minutes, whereas LFIA is restricted to single-analyte analysis. Precision metrics further distinguished the two methods: BiZi-FIA exhibited a maximum relative error of 7.8% across dilution series, while LFIA produced non-quantitative outputs below its detection threshold (Table 3). The results show that BiZi-FIA achieved superior sensitivity and a wider dynamic range, particularly for low-concentration samples. In contrast, the BiZi-FIA system continued to reliably detect IgE at these low concentrations, underscoring its advantage in detecting low-abundance biomarkers.

Furthermore, the ability to maintain such strong linear relationships under both triplicate and single-run configurations highlights the robustness of the system, making it highly suitable for rapid and multiplex biomarker detection in clinical and research applications. These results strongly support the potential of the system for detecting and quantifying proteins in complex biological matrices with exceptional accuracy and reproducibility.

Both biomarkers demonstrated excellent linearity with fitted curves closely aligning with experimental data points, as shown in FIGS. 9A-9H. These findings underscore the capability of the BiZi-FIA platform for precise detection. The ability to consistently produce reliable measurements across multiple replicates and under different conditions demonstrates its suitability for clinical and research applications requiring ultra-sensitive and precise protein quantification.

The measure relative error (R-Error) and dilution relative error (R-Error) were calculated to assess the precision of the two platforms. The measure R-Error is defined as the percentage difference between the BiZi-FIA measured value and the LFIA (I-IMMUNDX™) measured value. The dilution R-Error refers to the error introduced during the sample dilution process and is calculated similarly:

$$Relative Error = ( Measured Value - Expected Value \vert ) / ( Expected Value ) \times 100 \%$$

Additionally, under identical dilution conditions, the relative error increases as the sample concentration decreases. This is primarily attributed to sampling variability and Poisson noise introduced during the dilution process, which leads to greater measurement uncertainty at lower concentrations. As the concentration decreases, the proportional effect of even small variations in the sample or measurement process becomes more significant, contributing to an increase in relative error. However, despite the 32-fold dilution of the 2.2 μL tear sample, the overall error below 8%.

The results, shown in FIG. 9J, reveal a wide dynamic range for IgE, with concentrations spanning several orders of magnitude, while TNF-α, and NFL displayed relatively stable levels across cases. This ability to detect proteins with both highly variable and consistent baseline concentrations underscores the robustness and adaptability of the system for diverse biomarker analysis.

The findings highlight the BiZi-FIA technical strengths, including its broad dynamic range, sensitivity, and multiplexing capabilities. These results are particularly beneficial for the analysis of biomarkers in limited biological samples. However, these results are based on controlled experimental conditions and do not imply clinical conclusions. Future studies, including rigorously designed clinical trials, are needed to further evaluate the diagnostic potential and clinical utility.

TABLE 3
Raw data for IgE concentrations measured by LFIA and BiZi-FIA, along with their respective relative error rates. Also included are
the raw data and 32-fold dilution data for IgE concentrations measured by BiZi-FIA, accompanied by their relative error rates.

| Cases | 1 | 2 | 3 | 4 | 5 | 6 | 7 | 8 | 9 | 10 | Unit |
|---|---|---|---|---|---|---|---|---|---|---|---|
| LFIA | 46.77 | 8.56 | 3.6 | 1.28 | <1 | <1 | <1 | <1 | <1 | <1 | IU/mL |
| (Raw) | | | | | | | | | | | |
| BiZi-FIA | 486945 | 87932 | 38825 | 13031 | 11194 | 4039 | 1832 | 1097 | 828 | 486 | fM |
| (Raw) | | | | | | | | | | | |
| BiZi-FIA | 15388 | 2826 | 1184 | 433 | 329 | 120 | 53 | 32 | 24 | 14 | fM |
| ( 1/32) | | | | | | | | | | | |
| Measure | 0.8% | 2.1% | 2.7% | 3.0% | — | — | — | — | — | — | % |
| R-Error | | | | | | | | | | | |
| Dilution | 1.1% | 2.8% | 2.4% | 6.3% | 5.9% | 5.2% | 7.4% | 6.7% | 7.2% | 7.8% | % |
| R-Error | | | | | | | | | | | |

### SUMMARY

In conclusion, a BiZi-FIA system has been developed to provide high-density, high-throughput ultrabright single-molecule detection.

In an exemplary method, 40 μL solution with 450,000 microbeads labeled with QD were randomly placed in access conduits and transported to the array chambers, where microbeads were trapped by the height limit of the array chamber with 80% coverage and ˜90% High-capture efficiency of microbeads which significantly Improved the accuracy and Reduce the time for imaging. The detection of TNF a, LCN-1, COVid-19 N protein with single-molecule resolution showed excellent reliability.

Compared with traditional digital ELISA, digital IAC can achieve limit of detection in the attomolar range, an approximately 8-fold improvement over the commercial Simoas and Quanterix. This single-layer BiZi-FIA chip for ultrafast trapping of microbead arrays was developed, which can enhance efficiency and less congestion. The process is simple and controllable.

The described BiZi-FIA chip was fabricated using standard photolithography and soft lithography methods for samples as low as 0.22 μL with detection sensitivity at single molecular level within only 120 seconds. The linearity of single-molecule detection reached 98%. The sample volume required is 50 μL. In this experiment, to leverage the BiZi-FIA chip for digital immunoassay detection, the standard ELISA techniques were extended to detect ultralow-specific target biomarker. Due to the highly independent operating mode of the networked trap units, the device can be easily scaled up further and form more complicated patterns while retaining its effective microbead-trapping performance.

In conclusion, the BiZi-FIA system provides high-density, high-throughput ultrabright single-molecule detection. 40 μL solution with 200,000 microbeads labeled with QD were randomly placed in access conduits and transported to the array chambers, where microbeads were trapped by the height limit of the array chamber with 75% coverage and ˜90% High-capture efficiency of microbeads which significantly Improved the accuracy and reduce the time for imaging.

The detection of TNF-α and LCN-1 with single-molecule resolution showed excellent reliability. Compared with traditional digital ELISA, BIZI-FIA can achieve limit of detection in the attomole range, an approximately 8-fold improvement over the commercial Simoas and Quanterix.

Multiple biomarkers with ultra-low concentration of small volume samples can be detected by multilayer BiZi-FIA within a single biological sample with small volume (e.g., human tears). Bead-based and wall-based BiZi-FIA systems are developed for comparing the performance of sensitivity and reliability, which may effectively avoid fluorescent signal occlusion by conventional magnetic particles.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present methods which will be limited only by the appended claims.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different compositions and methods of use thereof does not indicate that the listed compositions and methods are obvious one to the other, nor is it an admission of equivalence or obviousness.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Aydin, S., A short history, principles, and types of ELISA, and our laboratory experience with peptide/protein analyses using ELISA. Peptides, 2015. 72: p. 4-15.
2. Wilson, D. H., et al., The Simoa HD-1 analyzer: a novel fully automated digital immunoassay analyzer with single-molecule sensitivity and multiplexing. Journal of laboratory automation, 2016. 21 (4): p. 533-547.
3. Dong, R., N. Yi, and D. Jiang, Advances in single molecule arrays (SIMOA) for ultra-sensitive detection of biomolecules. Talanta, 2024. 270: p. 125529.
4. Pérez-Ruiz, E., et al., Digital ELISA for the quantification of attomolar concentrations of Alzheimer's disease biomarker protein Tau in biological samples. Analytica chimica acta, 2018. 1015: p. 74-81.
5. Stephens, A. D., et al., Miniaturized microarray-format digital ELISA enabled by lithographic protein patterning. Biosensors and Bioelectronics, 2023. 237: p. 115536.
6. O'Connell, G. C., et al., Use of high-sensitivity digital ELISA improves the diagnostic performance of circulating brain-specific proteins for detection of traumatic brain injury during triage. Neurological research, 2020. 42 (4): p. 346-353.
7. Song, Y., et al., Rapid single-molecule digital detection of protein biomarkers for continuous monitoring of systemic immune disorders. Blood, The Journal of the American Society of Hematology, 2021. 137 (12): p. 1591-1602.
8. Song, L., et al., A digital enzyme-linked immunosorbent assay for ultrasensitive measurement of amyloid-β 1-42 peptide in human plasma with utility for studies of Alzheimer's disease therapeutics. Alzheimer's research & therapy, 2016. 8: p. 1-15.
9. Hsu, W., et al., Bead number effect in a magnetic-beads-based digital microfluidic immunoassay. Biosensors, 2022. 12 (5): p. 340.
10. Alnaimat, F., et al., Microfluidics Based Magnetophoresis: A Review. Chem Rec, 2018. 18 (11): p. 1596-1612.
11. Cohen, L., et al., Single molecule protein detection with attomolar sensitivity using droplet digital enzyme-linked immunosorbent assay. ACS nano, 2020. 14 (8): p. 9491-9501.
12. Anna, S. L., N. Bontoux, and H. A. Stone, Formation of dispersions using “flow focusing” in microchannels. Applied Physics Letters, 2003. 82 (3): p. 364-366.
13. Kan, C. W., et al., Digital enzyme-linked immunosorbent assays with sub-attomolar detection limits based on low numbers of capture beads combined with high efficiency bead analysis. Lab on a Chip, 2020. 20 (12): p. 2122-2135.
14. Zhang, Y. and H. Noji, Digital bioassays: theory, applications, and perspectives. Analytical chemistry, 2017. 89 (1): p. 92-101.
15. Yue, X., et al., Breaking through the Poisson Distribution: A compact high-efficiency droplet microfluidic system for single-bead encapsulation and digital immunoassay detection. Biosensors and Bioelectronics, 2022. 211: p. 114384.
16. Akama, K., K. Shirai, and S. Suzuki, Highly sensitive multiplex protein detection by droplet-free digital ELISA. Electronics and Communications in Japan, 2019. 102 (2): p. 43-47.
17. Wu, C., P. M. Garden, and D. R. Walt, Ultrasensitive detection of attomolar protein concentrations by dropcast single molecule assays. Journal of the American Chemical Society, 2020. 142 (28): p. 12314-12323.
18. Wu, C., A. M. Maley, and D. R. Walt, Single-molecule measurements in microwells for clinical applications. Critical Reviews in Clinical Laboratory Sciences, 2020. 57 (4): p. 270-290.
19. Ng, A. H., et al., Digital microfluidic magnetic separation for particle-based immunoassays. Analytical chemistry, 2012. 84 (20): p. 8805-8812.
20. Doonan, S., Microfluidic Technologies for Bioanalytical Chemistry: Advancing Epigenetic Profiling via Chromatin Immunoprecipitation in Droplets. 2019.
21. Rissin, D. M., et al., Multiplexed single molecule immunoassays. Lab on a Chip, 2013. 13 (15): p. 2902-2911.
22. Rissin, D. M., et al., Single-molecule enzyme-linked immunosorbent assay detects serum proteins at subfemtomolar concentrations. Nature biotechnology, 2010. 28 (6): p. 595-599.
23. Mohammed, H. A., The effect of different inlet geometries on laminar flow combined convection heat transfer inside a horizontal circular pipe. Applied Thermal Engineering, 2009. 29 (2-3): p. 581-590.
24. Yang, S., et al., A fractal analysis of laminar flow resistance in roughened microchannels. International Journal of Heat and Mass Transfer, 2014. 77: p. 208-217.
25. Rumi, M. and J. W. Perry, Two-photon absorption: an overview of measurements and principles. Advances in Optics and Photonics, 2010. 2 (4): p. 451-518.
26. Aleem, S. H. E. A., A. F. Zobaa, and M. M. A. Aziz, Optimal $ C $-type passive filter based on minimization of the voltage harmonic distortion for nonlinear loads. IEEE Transactions on Industrial Electronics, 2011. 59 (1): p. 281-289.
27. Komiyama, S., Single-photon detectors in the terahertz range. IEEE Journal of selected topics in quantum electronics, 2010. 17 (1): p. 54-66.
28. LECHNER, M., P. WOJNAR, and B. REDL, Human tear lipocalin acts as an oxidative-stress-induced scavenger of potentially harmful lipid peroxidation products in a cell culture system. Biochemical Journal, 2001. 356 (1): p. 129-135.
29. Lin, C.-W., et al., Elevated α-synuclein and NfL levels in tear fluids and decreased retinal microvascular densities in patients with Parkinson's disease. Geroscience, 2022. 44 (3): p. 1551-1562.
30. Agrawal, O. P., Formulation of Euler-Lagrange equations for fractional variational problems. Journal of Mathematical Analysis and Applications, 2002. 272 (1): p. 368-379.

We claim:

1. A microfluidic chip comprising:
a microfluidic platform comprising one or more microfluidic flow paths, wherein at least one of the microfluidic flow paths comprises an inlet conduit, a flow-trap junction (FTJ) structure, and an outlet conduit, wherein the at least one microfluidic flow path is configured for movement of fluid from the inlet conduit into the FTJ structure, and from the FTJ structure into the outlet conduit,
wherein the inlet conduit is wider where the fluid moves from the inlet conduit into the FTJ structure than where the fluid is introduced into the inlet conduit, wherein the inlet conduit contains a multiplicity of hydrophobic micropillar structures,
wherein the FTJ structure comprises a flow layer and a trap layer, wherein the flow layer is in contact with, on top of, and overlapping with the trap layer,
wherein the flow layer comprises a plurality of flow microfluidic channels each comprising a top, side walls, and an opening on the bottom, wherein the surfaces of the flow microfluidic channels are hydrophobic, wherein the flow microfluidic channels allow free passage of micro-scale particles and nano-scale objects, wherein the fluid flows in the same direction in all of the flow microfluidic channels,
wherein the flow microfluidic channels are parallel to each other,
wherein the trap layer comprises a plurality of trap microfluidic channels each comprising a bottom, side walls, and an opening on the top, wherein the trap microfluidic channels are parallel to each other, wherein the surfaces of the trap microfluidic channels are hydrophilic, wherein the fluid flows in the same direction in all of the trap microfluidic channels,
wherein the flow microfluidic channels are not parallel to the trap microfluidic channels, wherein the flow microfluidic channels and the trap microfluidic channels allow fluid movement from the flow microfluidic channels into the trap microfluidic channels via the openings on the bottom and openings on the top, respectively,
wherein the trap microfluidic channels, the transition from the flow microfluidic channels to the trap microfluidic channels, or a combination of both the trap microfluidic channels and the transition from the flow microfluidic channels to the trap microfluidic channels are configured to allow passage of the nano-scale objects through the trap microfluidic channels, whereby the passaged nano-scale objects flow into the outlet conduit,
wherein the trap microfluidic channels, the transition from the flow microfluidic channels to the trap microfluidic channels, or a combination of both the trap microfluidic channels and the transition from the flow microfluidic channels to the trap microfluidic channels are configured to trap the micro-scale particles in the trap microfluidic channels, whereby the trapped micro-scale particles, in combination, form an array within the FTJ structure.

2. The chip of claim 1, wherein each flow microfluidic channel comprises a terminus region proximal to the outlet conduit, wherein the terminus region comprises a curvature of the flow microfluidic channel, wherein the curvature directs fluid flow out of the microfluidic channel in the opposing direction to the directional fluid flow within the outlet conduit; and
wherein the opposing directional fluid flow provides resistance in the flow microfluidic channels and drives fluid movement laterally through the FTJ.

3. The chip of claim 1, wherein the side walls of the trap microfluidic channels are of uniform width and parallel to each other, wherein the height of the trap microfluidic channels is less than the diameter of the micro-scale particles.

4. The chip of claim 3, wherein the height of the trap microfluidic channels is more than the diameter or long dimension of the nano-scale objects.

5. The chip of claim 1, wherein the side walls of the trap microfluidic channels are not of uniform width, such that the trap microfluidic channels vary in width in a regular pattern along their lengths, wherein the pattern of width variation forms narrowings in the width of the trap microfluidic channels, wherein the width of the narrowings are less than the diameter of the micro-scale particles, wherein the width of the narrowings are more than the diameter or long dimension of the nano-scale objects.

6. The chip of claim 5, wherein all or a subset of the narrowings overlap the flow layer between some or each of the openings on the bottoms of adjacent flow microfluidic channels.

7. The chip of claim 5, wherein all or a subset of the narrowings overlap the opening on the bottom of some or each of the flow microfluidic channels.

8. The chip of claim 5, wherein a subset of the narrowings overlap the opening on the bottom of some or each of the flow microfluidic channels and a subset of the narrowings overlap the flow layer between some or each of the openings on the bottoms of adjacent flow microfluidic channels.

9. The chip of claim 5, wherein a subset of the narrowings overlap the opening on the bottom of each of the flow microfluidic channels and a subset of the narrowings overlap the flow layer between each of the openings on the bottoms of adjacent flow microfluidic channels.

10. The chip of claim 7, wherein the narrowings overlapping the openings on the bottoms of the flow microfluidic channels form a small trap entrance on a down-flow side of the opening and a large trap entrance on a down-flow side of the opening for alternating trap microfluidic channels, wherein the size of the small trap entrance is less than the diameter of the micro-scale particles, wherein the size of the small trap entrance is more than the diameter or long dimension of the nano-scale objects, and wherein the size of the large trap entrance is more than the diameter of the micro-scale particles.

11. The chip of claim 1, wherein the flow microfluidic channels and the trap microfluidic channels are at a right angle to each other.

12. The chip of claim 1, wherein the flow microfluidic channels and the trap microfluidic channels are at an oblique angle to each other.

13. The chip of claim 1, wherein the flow microfluidic channels and the trap microfluidic channels are at an angle of between 60° to 90°, between 60° to 90°, between 60° to 90°, between 60° to 90°, between 60° to 90°, between 70° to 90°, between 80° to 90°, between 85° to 90°, between 87° to 90°, between 88° to 90°, or between 89° to 90°, to each other.

14. The chip of claim 1, wherein the side walls of the trap microfluidic channels are angled toward the up-flow ends of the flow microfluidic channels.

15. The chip of claim 1, wherein the microfluidic flow path further comprises a sample inlet, wherein the microfluidic flow path is configured for movement of fluid from the sample inlet into the inlet conduit.

16. The chip of claim 1, wherein the microfluidic flow path further comprises a plurality of outlet channels, wherein the microfluidic flow path is configured for movement of fluid from the trap microfluidic channels into the outlet channels and from the outlet channels into the outlet conduit.

17. The chip of claim 16, wherein each trap microfluidic channel is flowably connected to a different one of the outlet channels.

18. The chip of claim 1, wherein the flow layer of the FTJ structure further comprises a plurality of outlet channels, wherein the outlet channels are interspersed between and parallel to the flow microfluidic channels, wherein the outlet channels each comprise a top, side walls, and an opening on the bottom, wherein the outlet channels and the trap microfluidic channels allow fluid movement from the trap microfluidic channels into the outlet channels via the opening on the bottom and openings on the top, respectively, wherein the microfluidic flow path is configured for movement of fluid from the trap microfluidic channels into the outlet channels and from the outlet channels into the outlet conduit.

19. The chip of claim 18, wherein the outlet channels alternate with the flow microfluidic channels in the flow layer of the FTJ structure.

20. The chip of claim 1, wherein the flow layer of the FTJ structure further comprises an outlet channel, wherein the outlet channel comprises a top, side walls, and an opening on the bottom, wherein the outlet channel overlaps the down-flow ends of the trap microfluidic channels, wherein the outlet channel and the trap microfluidic channels allow fluid movement from the trap microfluidic channels into the outlet channel via the opening on the bottom and openings on the top, respectively, wherein the microfluidic flow path is configured for movement of fluid from the trap microfluidic channels into the outlet channel and from the outlet channel into the outlet conduit.

21. A method for detecting a target biomarker in a fluid sample, the method comprising:
(a) introducing the fluid sample to one or more of the microfluidic flow paths of the microfluidic chip of claim 1, wherein the fluid sample comprises, or is bought into contact with after its introduction, a plurality of micro-scale particles and a plurality of nano-scale objects, and
(b) performing digital chromatography on the chip,
wherein the digital chromatography identifies the presence and/or quantity of the target biomarker in the fluid sample.

22. The method of claim 21, wherein step (a) further comprises introducing to a different microfluidic flow path of the same chip a control sample comprising a known amount of the target biomarker.

23. The method of claim 22, wherein the performance of digital chromatography of step (b) comprises actuating movement of fluid through the microfluidic flow paths in the microfluidic chip, wherein the movement filters and washes the micro-scale particles within the FTJ structure.

24. The method of claim 23, wherein the filtering of the micro-scale particles in the FTJ structure traps, and forms an array of, the micro-scale particles within the FTJ structure.

25. The method of claim 24, wherein step (b) further comprises imaging the array of micro-scale particles within the microfluidic chip.

26. The method of claim 21, wherein the micro-scale particles comprises a microbead.

27. The method of claim 26, wherein the microbead comprises a magnetic microbead.

28. The method of claim 21, wherein the micro-scale particles further comprise a first capture agent specific for a target biomarker.

29. The method of claim 28, wherein step (b) further comprises detecting and measuring the target biomarkers bound to the first capture agents on the micro-scale particles within the array.

30. The method of claim 28 further comprising, prior to step (a),
(i) incubating the fluid sample with the micro-scale particles for a time and in an amount effective for binding of the target biomarkers to the first capture agent; and
(ii) optionally washing the micro-scale particles.

31. The method of claim 30 further comprising, prior to step (a), contacting the micro-scale particles with the nano-scale objects for a time and in an amount effective for binding of the target biomarkers to the second capture agent.
